(19) United States
(12) Patent Application Publication
(10) Pub. No.: US 2025/0074972 A1
(43) Pub. Date: Mar. 6, 2025

(54) ANTIBODIES AND METHODS FOR GENERATING THE SAME

(71) Applicant: THE UNIVERSITY COURT OF THE UNIVERSITY OF ABERDEEN, Aberdeen (GB)

(72) Inventors: Andrew PORTER, Aberdeen (GB); Soumya PALLIYIL, Aberdeen (GB); Carol MUNRO, Aberdeen (GB)

(21) Appl. No.: 18/566,570

(22) PCT Filed: May 31, 2022
(86) PCT No.: PCT/EP2022/064798

§ 371 Date: Dec. 1, 2023

(30) Foreign Application Priority Data
Jun. 4, 2021 (GB) 2108055.1

(51) Int. Cl. C07K 16/14; A61K 39/00; A61P 31/10; C07K 14/38; C07K 14/40; G01N 33/50; G01N 33/569; G01N 33/68

(52) U.S. Cl. CPC C07K 16/14; A61P 31/10; C07K 14/38; C07K 14/40; G01N 33/5026; G01N 33/56961; G01N 33/6842; A61K 2039/505; G01N 2333/38; G01N 2333/40; G01N 2800/26

(57) ABSTRACT

Provided are methods for identifying surface-exposed epitopes of fungal cell wall proteins, and related peptide antigens, suitable for the development of antifungal antibodies. The methods allow for the detection of surface-exposed epitopes that are particularly highly expressed in drug-resistant fungal pathogens, and in pathogens exposed to antifungal drugs. Also provided are antifungal antibodies which may be derived by these methods. The methods and antibodies find use in the treatment of treating and diagnosing fungal infections, such as candidiasis, aspergillosis cryptococcosis, and Mucormycosis.

### SEQUENCE LISTING STATEMENT

A sequence listing accompanies this application and is submitted as an ASCII text file named “PCTEP2022064798_ST25_Sequence Listing.txt” which is 182,074 bytes in size and was created on Jul. 29, 2022. The sequence listing is electronically submitted via Patent Center with the application and is incorporated herein by reference in its entirety.

### TECHNICAL FIELD

The present invention relates generally to antibodies to surface exposed epitopes of the cell wall proteins of fungal pathogens, and strategies for their generation.

### BACKGROUND ART

Pathogenic fungi including members of the Aspergillus, Candida and Cryptococcus species cause life threatening infections in humans and kill more than a million people annually. Individuals with a weakened immune system or underlying health conditions have an increased risk of developing serious fungal infections such as invasive Candidiasis, Aspergillosis and Cryptococcosis. Population-based surveillance studies showed that the yearly incidence rate of invasive Candida infections by Candida albicans and related species including C. parapsilosis, C. glabrata and C. tropicalis is 8 per 100,000 of the population. Vaginal candidiasis affects 75% of women at least once during their reproductive age. C. auris, an emerging multidrug resistant fungal species which is difficult to identify and has 30-60% mortality rates, has been listed as an urgent threat in the CDC's 2019 Antibiotic Resistance Threats Report and is causing hospital outbreaks associated with COVID-19.

At present, there is a dramatic increase in the mortality and morbidity associated with invasive fungal infections (IFD) and currently used antifungal agents such as caspofungin, amphotericin B, and fluconazole have their own limitations. Development of resistance to azoles and echinocandins and the intrinsic ability of some fungal species to not respond to drugs warrant the need to develop novel alternative therapeutic approaches to tackle these life-threatening infections.

In fungi, the cell wall is a dynamic structure which is continuously changing in response to culture conditions, environmental stresses and the infection process. This makes identifying appropriate targets for generating therapeutic binders, such as antibodies, challenging because in vitro conditions that modulate cell wall protein expression and accessibility to drugs may not be replicated in an in vivo setting and vice versa.

It can be seen that novel sources of therapeutic agents for the treatment, targeting, detection and diagnosis of fungal pathogens and fungal infections, and methods for their generation, would make a contribution to the art.

### DISCLOSURE OF THE INVENTION

Aspects of the invention were based, in part, on studying variations in the cell wall proteome of Candida isolates and subsequent identification of surface exposed protein epitopes.

Using the novel methods disclosed herein, the inventors performed cell wall proteomic studies of C. albicans clinical isolate strains and identified several covalently linked cell wall proteins (“CWPs”) that are accessible for trypsin digestion and therefore surface exposed, and that, in some cases, are particularly highly expressed in drug-resistant fungal pathogens, and in pathogens exposed to antifungal drugs.

The epitopes of these CWPs are believed to be useful for raising antibodies which will neutralise the fungal species. Further, antibodies resulting from such strategies may find use in the treatment, prevention, diagnosis and/or detection of a variety of fungal infections such as candidiasis, aspergillosis cryptococcosis, and Mucormycosis. The suitability of such antibodies for such uses are demonstrated by their surprising and advantageous in vitro and in vivo properties, as disclosed herein. In particularly preferred embodiments, selection of candidate CWPs for raising antibodies is based on combining cell wall proteomic studies with other criteria such as whether CWP expression occurs in vivo during an infection, whether the CWPs are pathogen specific or conserved in multiple species; or whether the CWPs have a function related to building or maintaining a robust cell wall.

In addition, by performing profiling of fungal RNA, extracted from infected kidneys and harvested from a systemic infection model, the inventors identified several glycoprotein genes, such as Pga31, that are highly expressed in vivo yet poorly expressed under laboratory growth conditions.

### Methods for Identifying Surface-Exposed Epitopes of Fungal CWPs

In one aspect the invention provides a method for identifying epitopes of fungal cell wall proteins (“CWPs”), comprising:
(a) providing a population of a first strain of a fungal species;
(b) culturing:
   (i) a first sub-population of the first strain in the presence of an antifungal agent, thereby forming a first culture;
   (ii) a second sub-population of the first strain in the absence of an antifungal agent, thereby forming a second culture;
(c) harvesting the cells and isolating the cell walls from each culture;
(d) digesting the cell walls from each culture with a digesting agent for surface-exposed CWPs, thereby forming digested surface-exposed fungal CWPs;
(e) performing proteomic analysis on the digested surface-exposed fungal (e) CWPs; and
(f) identifying surface-exposed epitopes of the digested surface-exposed CWPs that are expressed in higher abundance in the first culture compared to the second culture.

In some embodiments, the first strain is resistant to an antifungal drug. In some embodiments, the first strain is susceptible to an antifungal drug. In some embodiments, the first strain is not resistant, or is classed as “intermediate”, with respect to an antifungal drug.

In some embodiments, the method comprises:
(a) providing populations of the first strain and a second strain of a fungal species, wherein the first strain is resistant to an antifungal drug, and the second strain is not resistant to an antifungal drug;
(b) culturing:
   (i) a first sub-population of the first strain in the presence of an antifungal agent, thereby forming a first culture;
   (ii) a second sub-population of the first strain in the absence of an antifungal agent, thereby forming a second culture;
   (iii) a first sub-population of the second strain in the presence of an antifungal agent, thereby forming a third culture; and
   (iv) a second sub-population of the second strain in the absence of an antifungal agent, thereby forming a fourth culture;
(c) harvesting the cells and isolating the cell walls from each culture;
(d) digesting the cell walls from each culture with a digesting agent for surface-exposed CWPs, thereby forming digested surface-exposed fungal CWPs;
(e) performing proteomic analysis on the digested surface-exposed fungal CWPs; and
(f) identifying surface-exposed epitopes of the digested surface-exposed CWPs that are:
   (i) expressed in higher abundance in the first culture compared to the second culture; and/or
   (ii) expressed in higher abundance in the third culture compared to the fourth culture.

The invention also provides a method for identifying epitopes of fungal cell wall proteins (“CWPs”), comprising:
(a) providing populations of a first strain and a second strain of a fungal species, wherein the first strain is resistant to an antifungal drug, and the second strain is not resistant to an antifungal drug;
(b) culturing:
   (i) a first sub-population of the first strain in the presence of an antifungal agent, thereby forming a first culture;
   (ii) a second sub-population of the first strain in the absence of an antifungal agent, thereby forming a second culture;
   (iii) a first sub-population of the second strain in the presence of an antifungal agent, thereby forming a third culture; and
   (iv) a second sub-population of the second strain in the absence of an antifungal agent, thereby forming a fourth culture;
(c) harvesting the cells and isolating the cell walls from each culture;
(d) digesting the cell walls from each culture with a digesting agent for surface-exposed CWPs, thereby forming digested surface-exposed fungal CWPs;
(e) performing proteomic analysis on the digested surface-exposed fungal CWPs; and
(f) identifying surface-exposed epitopes of the digested surface-exposed CWPs that are expressed in higher abundance in the first culture compared to the third culture.

In some embodiments, step (f) of the disclosed methods comprise:
(f) identifying surface-exposed epitopes of the digested surface-exposed CWPs that are, or are determined to be, involved in cell wall remodelling pathways and/or expressed in vivo during an infection; and are expressed in higher abundance in the first culture compared to the second culture;
or
(f) identifying surface-exposed epitopes of the digested surface-exposed CWPs that are, or are determined to be, involved in cell wall remodelling pathways and/or expressed in vivo during an infection; and
   (i) expressed in higher abundance in the first culture compared to the second culture; and/or
   (ii) expressed in higher abundance in the third culture compared to the fourth culture;
or
(f) identifying surface-exposed epitopes of the digested surface-exposed CWPs that are, or are determined to be, involved in cell wall remodelling pathways and/or expressed in vivo during an infection; and are expressed in higher abundance in the first culture compared to the third culture.

In some cases, the CWPs are identified, or identifiable, as being involved in cell wall remodelling pathways (for example the PKC cell integrity pathway and the Calcium/calcineurin signalling pathway). In some cases, the CWPs may be downstream of signalling pathways.

In some cases, the CWPs are identified, or identifiable, as being expressed in vivo during an infection. Expression of the CWPs may occur in vivo during an infection. The infection may be an infection as described herein. Identification of this and other features may be performed using RNA expression. For example, RNA is isolated from infected tissues harvested from a systemic infection model (e.g. a murine model) and gene expression is measured by real time PCR.

In some cases, the CWPs are identified, or identifiable, as being pathogen specific; conserved across multiple strains of a pathogen species; or conserved across multiple species of pathogen. Identification of this and other features may be performed using bioinformatics analysis, for example by comparing the genomes of fungal species that are available in genome databases. Genome comparisons have been published; see e.g. Butler et al. (2009) Nature; 459 (7247): 657-62).

In some cases, the CWPs are identified, or identifiable, as having a function related to building or maintaining a robust cell wall, or maintaining cell wall integrity.

Identification of the above features can be performed by proteomic analysis, as described herein.

In some cases, differences in the expression levels of surface-exposed CWPs between different strains and culture conditions enables the identification of CWPs that are particularly advantageous for the development of novel antigens and high affinity antifungal antibodies.

For example, in some cases, the surface-exposed CWPs that are used for identifying epitopes using the disclosed methods, are those that are expressed in at least 2 times the abundance in a fungal strain that is resistant to an antifungal drug, compared to a fungal strain that is susceptible (or is not resistant, or is classed as “intermediate”, as defined herein) to an antifungal drug. In some cases, said CWPs are those that are expressed in at least 3, 4, 5, 6, 7, 8, 9 or 10 times the abundance. In other cases, the surface-exposed CWPs that are used for identifying epitopes using the disclosed methods, are those that are expressed in at least 2 times the abundance in a fungal strain that has been exposed to an antifungal drug, compared to the same strain that has not been exposed to an antifungal drug. In some cases, said CWPs are those that are expressed in at least 3, 4, 5, 6, 7, 8, 9 or 10 times the abundance.

In some particularly preferred embodiments, the surface-exposed CWPs that are used for identifying epitopes using the disclosed methods, are those that are expressed in a fungal strain that is resistant to an antifungal drug, but not expressed in a fungal strain that is susceptible (or is not resistant) to an antifungal drug. In some particularly preferred embodiments, the surface-exposed CWPs that are used for identifying epitopes using the disclosed methods, are those that are expressed in a fungal strain that has been exposed to antifungal drug, but not expressed in a fungal strain that has not been exposed to an antifungal drug.

In some cases, the antifungal agent is capable of inducing cell wall remodelling pathways.

In some cases, the strains or populations are cultured in the presence of an antifungal agent, wherein the antifungal agent is present in the culture at a sub-MIC (“minimum inhibitory concentration”) value. In some cases, the antifungal agent is present in the culture at a MIC value. In some cases, the antifungal agent is present in the culture above the MIC value.

The MIC value will be specific to the antifungal drug used as well as the fungal species or fungal strain used, and can be determined by the skilled person in a straightforward manner. Exemplary MIC concentrations are between 0.1 to 100 μg/mL, e.g. 1 to 50, 2 to 20, or 5 to 10 μg/mL.

The antifungal agent (or “antifungal drug”) may be selected from the group consisting of caspofungin, amphotericin B, fluconazole, azoles or echinocandins. In some preferred embodiments, the antifungal drug is caspofungin.

Harvesting of the cells from a culture may comprise centrifugation steps followed by wash steps. Isolation of cell walls from a culture may comprise mechanical breakage of the cells, wash steps to remove cytoplasmic contamination, and freeze-drying steps. Such steps may be achieved by techniques known in the art (see e.g. Kapteyn et al. 2000). In some preferred embodiments, a Fastprep machine and zirconia beads are used for mechanical breakage of the cell walls. In some preferred embodiments, the cell wall fractions are further cleaned by being placed at 4° C. in 1M NaCl overnight.

In some preferred embodiments, the digestion step comprises digesting the cell walls from each culture with trypsin to form tryptic cell wall fractions, wherein the fractions comprise surface-exposed CWPs. Digestion with trypsin may be performed according to the PRIME-XS protocol (“PRIME-XS Protocol NPC In Solution Digestion,” 2013). Nevertheless, the digestion step can also be performed with any suitable digesting agent, such as elastase, proteinase K, chymotrypsin, LysC, LysN, AspN, GluC and ArgC.

The proteomic analysis can allow for determination of expression levels of the digested surface-exposed CWPs. It may be carried out by any suitable method known in the art, e.g. using Proteome Discoverer 2.2 software (Thermo Fisher Scientific), with the CWPs matched from Candida Genome Database (www.candidagenome.org). In some cases, a cut-off of at least 2 peptides detected per protein is used. The Area Under the Curve (AUC) may give a measure of protein abundances allowing for expression levels to be determined.

Proteomic analysis may also be performed by employing liquid chromatography-tandem mass spectrometry (LC-MS/MS) of cell wall fractions.

### Design of Peptide Antigens

In a related aspect, the inventors have devised novel peptide antigens, and methods for their generation, that are particularly advantageous for the development of high affinity antifungal antibodies to surface-exposed CWPs.

Accordingly, the invention provides a method of generating a peptide antigen, comprising:
(a) providing a surface-exposed epitope of a fungal cell wall protein (“CWP”); and
(b) generating a peptide antigen derived from the epitope, wherein the derived antigen comprises an amino acid sequence that:
   (i) is comprised by the epitope, or has at least 70% sequence identity to the epitope; and
   (ii) has a hydropathy index value of less than 0 for more than 50% of the amino acids comprised by the amino acid sequence; and/or
   (iii) has 20% or more predicted ß-turn secondary structure.

In a related aspect, the invention provides use of a surface-exposed epitope of a fungal cell wall protein (“CWP”) in a method of generating a peptide antigen, comprising generating a peptide antigen derived from the epitope, wherein the derived antigen comprises an amino acid sequence that:
(i) is comprised by the epitope, or has at least 70% sequence identity to the epitope; and
(ii) has a hydropathy index value of less than 0 for more than 50% of the amino acids comprised by the amino acid sequence; and/or
(iii) has 20% or more predicted ß-turn secondary structure.

In another related aspect, the invention provides a peptide antigen derived from a surface-exposed epitope of a fungal cell wall protein (“CWP”), wherein the derived peptide antigen:
(i) consists of an amino acid sequence that is comprised by the epitope, or that has at least 20% sequence identity to the epitope; and
(ii) has a hydropathy index value of less than 0 for more than 50% of the amino acids comprised by the amino acid sequence of the peptide antigen; and/or
(iii) has 40% or more predicted ß-turn secondary structure.

In some cases, less than 50, 45, 40, 35, 30, 25, 20, 15, 10 or 5% of the amino acid residues of the amino acid sequence of the peptide antigen have a hydropathy index of more than 1.

In some cases, the peptide antigen has 25% or more, 30% or more, 35% or more, or 40% or more predicted ß-turn secondary structure. In some cases, the peptide antigen has between 40-45, 45-50, 50-55, 55-60, 60-65, 65-70, 70-75, 75-80, 80-85, 85-90, 90-95, or 95-100% predicted ß-turn secondary structure. In some preferred embodiments, the peptide antigen has between 40-45% predicted ß-turn secondary structure. In some preferred embodiments, the peptide antigen has between 80-85% predicted ß-turn secondary structure.

In another related aspect, the invention provides a method of producing an antifungal antibody that specifically bind to surface-exposed epitopes of fungal cell wall proteins (“CWPs”), comprising:
(i) providing a peptide antigen generated by a method of the invention, or providing a surface-exposed epitope of a fungal CWP identified by the method of the invention;
(ii) screening a human antibody library against the antigen or epitope; and
(iii) producing an antibody that specifically binds to the antigen or epitope.

In some preferred embodiments, the amino acid sequence of the peptide antigen has at least 70, 75, 80, 85, 90, 91, 92, 93, 94, 95, 96, 97, 98 or 99%, or 100%, sequence identity to the surface-exposed epitope of a fungal cell wall protein (“CWP”). The peptide antigen may comprise or consist of an amino acid sequence selected from SEQ ID NO: 77, 78, 79 or 80.

In some cases, the peptide antigen comprises an amino acid sequence having at least 75, 80, 85, 90, 91, 92, 93, 94, 95, 96, 97, 98 or 99%, or 100% selected from SEQ ID NO: 77, 78, or 79, or 80.

In some cases, the peptide antigen is between 10 and 50 amino acids in length. In some cases, the peptide antigen is between 15 and 40 amino acids in length. In some cases, the peptide antigen is between 20 and 35 amino acids in length. In some cases, the peptide antigen is between 25 and 35 amino acids in length. In some cases, the peptide antigen is between 27 and 33 amino acids in length. In some cases, the peptide antigen is 30 amino acids in length.

In some cases, the surface-exposed CWP epitope is one that is identified by the methods of the invention.

In some cases, the CWP is Pga31, Utr2 or Phr2. In other cases, the CWP is Cht2. In other cases, the CWP is Crh11, and Pga29. In some cases the CWP is a carbohydrate-active enzyme.

The term “hydropathy” (or “hydropathicity”) of an amino acid is a measure of the hydrophobic or hydrophilic properties of its sidechain. In a given peptide sequence, the presence of hydrophilic and hydrophobic amino acid residues influences its solubility in aqueous solutions and is one of the contributing parameters to the ‘antigenicity’ or ‘immunogenicity’ of that peptide. Hydropathy can be determined by any appropriate method known in the art (see e.g. Hodges 2006). A hydropathy, or hydropathy index, of less than 0 means that the amino acid has a hydrophilic side chain. A hydropathy, or hydropathy index, of more than 0 means that the amino acid has a hydrophobic side chain.

As used here, a ß-turn is a classical secondary structure found on the surface of proteins that causes a change in direction of the polypeptide chain. They are very common motifs in proteins and polypeptides, increasing molecular recognition and antibody reactivity. The percentage ß-turn structure of an amino acid sequence can be determined by any appropriate method known in the art, such as by circular dichroism.

Screening of the human antibody library against the peptide antigen can be accomplished by appropriate methods known in the art e.g. phage display technology. The screening may comprise biopanning of the library against decreasing stepwise concentrations of the epitope, in order to isolate high affinity binders from the recombinant antibody libraries.

The peptide antigen can be generated by any routine methods known in the art, such as solid-state synthesis, or by recombinant methods.

Also disclosed are methods for producing an antibody antigen-binding domain for a fungal antigen described herein, which methods comprise utilising or modifying one or more of the CDRs, FWs, VH and VL domains having sequences as disclosed herein.

In any of the aspects disclosed above, the CWP may be a glycoprotein, which may be post-translationally modified by the addition of glycosylphosphatidylinositol.

The cell wall of C. albicans is covered in an outer layer of glycoproteins that play an important role in pathogenesis by mediating interactions between the host and the fungus (Gow et al., 2019). Cell surface glycoproteins are important virulence factors—examples include invasins, adhesins and superoxide dismutases that combat the host's oxidative burst defences and are shed from the invading fungus during infection.

Antibodies against these proteins have been detected in patients' sera and therefore Candida glycoproteins, such as those identified herein, will have utility for vaccine generation and therapeutic antibody development. The major class of cell surface glycoproteins are post-translationally modified by the addition of Glycosylphosphatidylinositol (GPI)-anchor and can be plasma membrane localised or translocated into the cell wall where they are covalently attached to the β-(1,6)-glucan polymer.

Around 115 putative GPI-anchored proteins have been detected in C. albicans using in silico analysis (Plaine et al., 2008). Some of these have enzymatic functions associated with cell wall biosynthesis and cell wall remodeling.

The CWP may be covalently attached to β-(1,6)-glucan polymer within a fungal cell wall. The CWP may be an Aspergillus, Candida or Cryptococcus CWP.

In some embodiments, the fungal species is C. albicans, C. tropicalis, or C. auris. The fungal species may also be Candida glabrata, Candida parapsilosis (a clonal complex of three species-C. parapsilosis, C. orthopsilosis and C. metapsilosis), and Candida krusei (synonym: Issatchenkia orientalis). The fungal species may also be Candida guilliermondii, Candida lusitaniae, Candida kefyr, Candida famata (synonym: Debaryomyces hansenii), Candida inconspicua, Candida rugosa, Candida dubliniensis, Candida norvegensis, Candida haemulonii.

Nevertheless, the fungal species may also be an Aspergillus species, such as A. fumigatus, A. flavus, A. niger, A. terreus, A. nidulans, or A. oryzae.

The fungal species may also be a Cryptococcus species, such as C. gattii, C. deuterogattii, C. neoformans var grubii or C. neoformans var neoformans.

In some cases, the fungal species is C. albicans, and the strain that is resistant to an antifungal drug is K063-3, B15_004476, or B12_007355_1.

In some cases, the fungal species is C. albicans, and the strain that is susceptible to an antifungal drug is SC5314, CBS8758, ATCC2091, ATCC76615, B17_009053, or B17_008835.

In some cases, the fungal species is C. tropicalis, and the strain that is resistant to an antifungal drug is Ct2. In some cases, the fungal species is C. tropicalis, and the strain that is susceptible to an antifungal drug is Ct1.

Certain preferred embodiments of these aspects of the invention are also discussed in the Examples.

### Antifungal Antibodies

In a related aspect, the present invention provides an antibody that specifically binds to a surface-exposed fungal cell wall protein (“CWP”). In some embodiments, the antibody is an isolated antibody. In some embodiments, the antibody is a recombinant antibody. In some embodiments, the antibody is an isolated recombinant antibody.

In some embodiments, the antibody is known as an antifungal/antifungal antibody. In some preferred embodiments, the CWP is a glycoprotein that is post-translationally modified by the addition of glycosylphophatidylinositol. In some preferred embodiments the CWP is covalently attached to ß-(1,6)-glucan polymer within a fungal cell wall. The CWP may be an Aspergillus, Candida or Cryptococcus CWP.

In some preferred embodiments the antibody specifically binds to Pga31, Utr2 or Phr2. In some preferred embodiments, the antibodies specifically bind to an antigen produced or epitope identified by a method of the invention. In some cases, the CWP may have an amino acid sequence having at least 70%, more preferably one of at least 75%, 80%, 85%, 90%, 95%, 96%, 97%, 98%, 99%, or 100%, sequence identity to SEQ ID NO: 81, 82, 83 or 84.

Suitable antibodies to such antigens may be prepared by known techniques, for example those disclosed in “Monoclonal Antibodies: A manual of techniques”, H Zola (CRC Press, 1988) and in “Monoclonal Hybridoma Antibodies: Techniques and Applications”, J G R Hurrell (CRC Press, 1982). Chimeric antibodies are discussed by Neuberger et al. (1988, 8th International Biotechnology Symposium Part 2, 792-799).

In some preferred embodiments, the antibody:
(i) comprises an amino acid sequence selected from SEQ ID NO: 2 or 4, and specifically binds to Pga31;
(ii) comprises an amino acid sequence selected from SEQ ID NO: 6, 8, 10, 12, 14, 16, 18, 20, 22, 24, 26, 28 or 30, and specifically binds to Utr2; or
(iii) comprises an amino acid sequence selected from SEQ ID NO: 32, 34, 36, 38, 40, 42, 44, 46, 48, 50, 52, 54, 56, 58, 60, 62, 64, 66, 68, 70, 72, 74 or 76, and specifically binds to Phr2,
   or is a variant thereof having at least 70%, more preferably one of at least 75%, 80%, 85%, 90%, 95%, 96%, 97%, 98%, 99%, or 100%, sequence identity to said amino acid sequence, or is a variant thereof in which one or two or three amino acids in said amino acid sequence are replaced with another amino acid.

In some cases, the antibody comprises at least one heavy chain variable (“VH”) region incorporating one, two or all three of VH CDR1, VH CDR2 and VH CDR3 of:
(i) SEQ ID NO: 2 or 4;
(ii) SEQ ID NO: 6, 8, 10, 12, 14, 16, 18, 20, 22, 24, 26, 28 or 30; or
(iii) SEQ ID NO: 32, 34, 36, 38, 40, 42, 44, 46, 48, 50, 52, 54, 56, 58, 60, 62, 64, 66, 68, 70, 72, 74 or 76;
   or is a variant thereof having at least 70%, more preferably one of at least 75%, 80%, 85%, 90%, 95%, 96%, 97%, 98%, 99%, or 100%, sequence identity to said amino acid sequence, or is a variant thereof in which one or two or three amino acids in said amino acid sequence are replaced with another amino acid.

In some cases, the antibody comprises at least one light chain variable (“VL”) region incorporating one, two or all three of VL CDR1, VL CDR2 and VL CDR3 of:
(i) SEQ ID NO: 2 or 4;
(ii) SEQ ID NO: 6, 8, 10, 12, 14, 16, 18, 20, 22, 24, 26, 28 or 30; or
(iii) SEQ ID NO: 32, 34, 36, 38, 40, 42, 44, 46, 48, 50, 52, 54, 56, 58, 60, 62, 64, 66, 68, 70, 72, 74 or 76,
   or is a variant thereof having at least 70%, more preferably one of at least 75%, 80%, 85%, 90%, 95%, 96%, 97%, 98%, 99%, or 100%, sequence identity to said amino acid sequence, or is a variant thereof in which one or two or three amino acids in said amino acid sequence are replaced with another amino acid.

In some cases, the antibody comprises (a) at least one heavy chain variable (“VH”) region incorporating one, two or all three of VH CDR1, VH CDR2 and VH CDR3; and (b) at least one light chain (“VL”) variable region, each incorporating one, two or all three of VL CDR1, VL CDR2 and VL CDR3, of:
(i) SEQ ID NO: 2 or 4;
(ii) SEQ ID NO: 6, 8, 10, 12, 14, 16, 18, 20, 22, 24, 26, 28 or 30; or
(iii) SEQ ID NO: 32, 34, 36, 38, 40, 42, 44, 46, 48, 50, 52, 54, 56, 58, 60, 62, 64, 66, 68, 70, 72, 74 or 76;
   or is a variant thereof having at least 70%, more preferably one of at least 75%, 80%, 85%, 90%, 95%, 96%, 97%, 98%, 99%, or 100%, sequence identity to said amino acid sequence, or is a variant thereof in which one or two or three amino acids in said amino acid sequence are replaced with another amino acid.

In some cases, the antibody comprises at least one heavy chain variable region or at least one light chain variable region of:
(i) SEQ ID NO: 2 or 4;
(ii) SEQ ID NO: 6, 8, 10, 12, 14, 16, 18, 20, 22, 24, 26, 28 or 30; or
(iii) SEQ ID NO: 32, 34, 36, 38, 40, 42, 44, 46, 48, 50, 52, 54, 56, 58, 60, 62, 64, 66, 68, 70, 72, 74 or 76;
   or is a variant thereof having at least 70%, more preferably one of at least 75%, 80%, 85%, 90%, 95%, 96%, 97%, 98%, 99%, or 100%, sequence identity to said amino acid sequence, or is a variant thereof in which one or two or three amino acids in said amino acid sequence are replaced with another amino acid.

In some cases, the antibody comprises at least one heavy chain variable region and at least one light chain variable region of:
(i) SEQ ID NO: 2 or 4;
(ii) SEQ ID NO: 6, 8, 10, 12, 14, 16, 18, 20, 22, 24, 26, 28 or 30; or
(iii) SEQ ID NO: 32, 34, 36, 38, 40, 42, 44, 46, 48, 50, 52, 54, 56, 58, 60, 62, 64, 66, 68, 70, 72, 74 or 76;
   or is a variant thereof having at least 70%, more preferably one of at least 75%, 80%, 85%, 90%, 95%, 96%, 97%, 98%, 99%, or 100%, sequence identity to said amino acid sequence, or is a variant thereof in which one or two or three amino acids in said amino acid sequence are replaced with another amino acid.

The light and heavy chain CDRs 1-3 of the antibodies disclosed herein may also be particularly useful in conjunction with a number of different framework regions (“FRs”). Accordingly, in some cases, the antibody further comprises at least one heavy chain (“VH”) variable region incorporating one, two, three or all four of VH FR1, VH FR2, VH FR3 and VH FR4 of:
(i) SEQ ID NO: 2 or 4;
(ii) SEQ ID NO: 6, 8, 10, 12, 14, 16, 18, 20, 22, 24, 26, 28 or 30; or
(iii) SEQ ID NO: 32, 34, 36, 38, 40, 42, 44, 46, 48, 50, 52, 54, 56, 58, 60, 62, 64, 66, 68, 70, 72, 74 or 76;

In some further cases, the antibody further comprises at least one light chain variable (“VL”) region incorporating one, two, three or all four of VL FR1, VL FR2, VL FR3 and VL FR4 of:
(i) SEQ ID NO: 2 or 4;
(ii) SEQ ID NO: 6, 8, 10, 12, 14, 16, 18, 20, 22, 24, 26, 28 or 30; or
(iii) SEQ ID NO: 32, 34, 36, 38, 40, 42, 44, 46, 48, 50, 52, 54, 56, 58, 60, 62, 64, 66, 68, 70, 72, 74 or 76;

CDR and FR sequences are determined by Kabat definition.

The antibodies may be a single-chain antibody (scab). A scAb has a constant light (CL) chain fused to the VL chain of an scFv fragment. The CL chain is optionally the human kappa light chain (HuCκ).

The antibodies may be a single-chain variable fragment (scFv). An scFv fragment is a fusion of a variable heavy (VH) and variable light (VL) chain. A single chain Fv (scFv) may be comprised within a mini-immunoglobulin or small immunoprotein (SIP), e.g. as described in Li et al. (1997). An SIP may comprise an scFv molecule fused to the CH4 domain of the human IgE secretory isoform IgE-S2 (ε_{S2}-CH4; Batista, F. D., Anand, S., Presani, G., Efremov, D. G. and Burrone, O. R. (1996). The two membrane isoforms of human IgE assemble into functionally distinct B cell antigen receptors. J. Exp. Med. 184:2197-2205) forming a homo-dimeric mini-immunoglobulin antibody.

The antibodies may also include any polypeptide or protein comprising an antibody antigen-binding site described herein, including Fab, Fab₂, Fab₃, diabodies, triabodies, tetrabodies, minibodies and single-domain antibodies, as well as whole antibodies of any isotype or sub-class.

In some aspects, the invention provides a polynucleotide encoding the antibodies of the invention. In some cases, the polynucleotide comprises a sequence that has at least 70, 75, 80, 85, 90, 91, 92, 93, 94, 95, 96, 97, 98, 99% sequence identity to, or is selected from, SEQ ID NO: 1, 3, 5, 7, 9, 11, 13, 15, 17, 19, 21, 23, 25, 27, 29, 31, 33, 35, 37, 39, 41, 43, 45, 47, 449, 51, 53, 55, 57, 59, 61, 63, 65, 67, 69, 71, 73 or 75.

Also disclosed herein is a method for producing an antibody antigen-binding domain for a fungal antigen, the method comprising:
(i) providing, by way of addition, deletion, substitution or insertion of one or more amino acids in the amino acid sequence of a parent VH domain comprising HCDR1, HCDR2 and HCDR3, wherein the parent VH domain HCDR1, HCDR2 and HCDR3 have an amino acid sequence of those corresponding regions of an antibody described herein, a VH domain which is an amino acid sequence variant of the parent VH domain,
(ii) optionally combining the VH domain thus provided with one or more VL domains to provide one or more VH/VL combinations; and
(iii) testing said VH domain which is an amino acid sequence variant of the parent VH domain or the VH/VL combination or combinations to identify an antibody antigen binding domain for the fungal antigen.

Also disclosed is a method for producing an antibody that specifically binds to a fungal antigen, which method comprises:
(i) providing starting nucleic acid encoding a VH domain or a starting repertoire of nucleic acids each encoding a VH domain, wherein the VH domain or VH domains either comprise a HCDR1, HCDR2 and/or HCDR3 to be replaced or lack a HCDR1, HCDR2 and/or HCDR3 encoding region;
(ii) combining said starting nucleic acid or starting repertoire with donor nucleic acid or donor nucleic acids encoding or produced by mutation of the amino acid sequence of an HCDR1, HCDR2, and/or HCDR3 having the amino acid sequences of those corresponding regions of an antibody described herein, such that said donor nucleic acid is or donor nucleic acids are inserted into the CDR1, CDR2 and/or CDR3 region in the starting nucleic acid or starting repertoire, so as to provide a product repertoire of nucleic acids encoding VH domains;
(iii) expressing the nucleic acids of said product repertoire to produce product VH domains;
(iv) optionally combining said product VH domains with one or more VL domains;
(v) selecting an antibody that binds the fungal antigen, which antibody comprises a product VH domain and optionally a VL domain; and
(vi) recovering said antibody or nucleic acid encoding it.

An antibody as described herein may be one which binds selectively or preferentially to a drug-resistant fungal cell compared to a drug-susceptible fungal cell. For example, the antibody may display at least a 1.1, 1.5, 2, 2.5, 3, 3.5, 4, 4.5 or 5 fold increase in binding to a drug-resistant fungal cell compared to a drug-susceptible fungal cell. The drug may be caspofungin. Binding can be assessed as described herein with ELISA e.g. as in the Examples below.

An antibody as described herein may be one which binds selectively or preferentially to a fungal cell treated with an antifungal drug compared to an untreated fungal cell. For example, the antibody may display at least a 1.1, 1.5, 2, 2.5, 3, 3.5, 4, 4.5 or 5 fold increase in binding to a caspofungin-treated fungal cell compared to a non-treated fungal cell. Binding can be assessed as described herein with ELISA e.g. as in the Examples below.

An antibody as described herein may be one which prevents or reduces fungal biofilm formation. In some cases the fungal biofilm is a Candida biofilm, such as C. albicans or C. auris. In some cases, the antibody as described herein may be one which prevents or reduces fungal biofilm formation from between 1 to 200 μg/mL e.g. 2.5 to 160, 5 to 40, or 10 to 20 μg/mL. Biofilm formation can be assessed as described herein e.g. as in the Examples below. Relatedly, provided herein is a method of reducing or preventing biofilm formation of a fungal cell, for example C. albicans, the method comprising contacting or pre-incubating the fungal cell with an antibody as described herein.

An antibody as described herein may be one which increases the rate of opsonisation of a fungal cell, for example C. albicans. Relatedly, provided herein is a method of opsonising, or increasing the rate of opsonisation of a fungal cell, for example C. albicans, the method comprising contacting or pre-incubating the fungal cell with an antibody as described herein.

An antibody as described herein may be one which increases the rate of engulfment of a fungal cell, for example C. albicans, by macrophages. Relatedly, provided herein is method of increasing the rate of engulfment of a fungal cell, the method comprising contacting the fungal cell with an antibody as described herein.

An antibody as described herein may be one that increases the rate of macrophage attraction to a fungal cell, for example C. albicans. Relatedly, provided herein is a method of increasing the rate of macrophage attraction to a fungal cell, for example C. albicans, the method comprising contacting or pre-incubating the fungal cell with an antibody as described herein.

An antibody as described herein may be one which binds Pga31, Utr2 or Phr2 with an EC₅₀ values of 1 to 1500, e.g. 10 to 500, or 20 to 200 ng/ml.

An antibody as described herein may be one which binds to Pga31 with an EC₅₀ value of 10 nM or lower, 5 nM or lower, 4 nM or lower, 3 nM or lower, 2 nM or lower or 1 nM or lower. An antibody as described herein may be one which binds to Pga31 with an EC₅₀ value of between 100 pM-10 nM, 200 pM-5 nM, 250 pM-4 nM, 500 pM-3 nM, or 1 nM-2 nM. An antibody as described herein may be one which binds to Pga31 with an EC₅₀ value of about 10 nM, about 5 nM, about 4 nM, about 3 nM, about 2 nM, about 1 nM, or about 500 pM.

An antibody as described herein may be one which binds to Utr2 with an EC₅₀ value of 1-500 nM, 2-300 nM, 5-200 nM, 10-150 nM, or 50-100 nM. An antibody as described herein may be one which binds to Utr2 with an EC₅₀ value of about 10 nM, about 5 nM, about 4 nM, about 3 nM, about 2 nM, about 1 nM, or about 500 pM. An antibody as described herein may be one which binds to Utr2 with an EC₅₀ value of between 100 pM-10 nM, 200 pM-5 nM, 250 pM-4 nM, 500 pM-3 nM, or 1 nM-2 nM.

An antibody as described herein may be one which binds to Phr2 with an EC₅₀ value of 1-500 nM, 2-300 nM, 5-200 nM, 10-150 nM, or 50-100 nM. An antibody as described herein may be one which binds to Phr2 with an EC₅₀ value of about 10 nM, about 5 nM, about 4 nM, about 3 nM, about 2 nM, about 1 nM, or about 500 pM. An antibody as described herein may be one which binds to Phr2 with an EC₅₀ value of between 100 pM-10 nM, 200 pM-5 nM, 250 pM-4 nM, 500 pM-3 nM, or 1 nM-2 nM.

An antibody as described herein may be one which binds to Cht2 with an EC₅₀ value of 1-500 nM, 2-300 nM, 5-200 nM, 10-150 nM, or 50-100 nM. An antibody as described herein may be one which binds to Cht2 with an EC₅₀ value of about 10 nM, about 5 nM, about 4 nM, about 3 nM, about 2 nM, about 1 nM, or about 500 pM. An antibody as described herein may be one which binds to Cht2 with an EC₅₀ value of between 100 pM-10 nM, 200 pM-5 nM, 250 pM-4 nM, 500 pM-3 nM, or 1 nM-2 nM.

An antibody as described herein may be one which binds to a Candida cell with an EC₅₀ value of 200 nM or lower, 100 nM or lower, 60 nM or lower, 50 nM or lower, 40 nM or lower, 30 nM or lower, 20 nM or lower or 10 nM or lower. An antibody as described herein may be one which binds to a Candida cell with an EC₅₀ value of about 100 nM, about 60 nM, about 50 nM, about 40 nM, about 30 nM, about 20 nM, about 10 nM, or about 1 nM. An antibody as described herein may be one which binds to a Candida cell with an EC₅₀ value of between 1 nM-200 nM, 5 nM-150 nM, 10 nM-100 nM, 20 nM-80 nM, or 55 nM-65 nM.

An antibody as described herein may be one which competes with an antibody of the invention for binding to a peptide antigen of the invention, or a surface-exposed epitope of a cell wall protein as disclosed herein.

EC₅₀ can be assessed as described hereinafter e.g. with ELISA as described in the Examples below.

An antibody as described herein may be conjugated to a toxic payload (e.g. ricin) that could kill the fungus and act as a therapeutic antibody. The toxic payload may also comprise or consist of existing antifungal agents such as echinocandins, polyenes, azoles or their structural analogues, antifungal peptides, antifungal exotoxins and other cell killing agents.

Also provided herein are compositions comprising an antibody as described herein and a drug. Also provided herein are compositions comprising an antibody as described herein, a drug, and a linker between the antibody and the drug (“antibody-drug conjugate”). In some preferred embodiments the drug is an antifungal drug or antifungal agent as described herein. The off-target toxicity of the payload may be reduced using an ADC and delivering the payload specifically into the fungus, for example the cell wall of the fungus.

It is known that there are a large number of Candida species. Key Candida species which may be targeted by the antibodies described herein include Candida albicans, Candida glabrata, Candida tropicalis, Candida parapsilosis (a clonal complex of three species-C. parapsilosis, C. orthopsilosis and C. metapsilosis), and Candida krusei (synonym: Issatchenkia orientalis). Less-prominent species include Candida guilliermondii, Candida lusitaniae, Candida kefyr, Candida famata (synonym: Debaryomyces hansenii), Candida inconspicua, Candida rugosa, Candida dubliniensis, Candida norvegensis, Candida auris, Candida haemulonii.

It is known that there are a large number of Aspergillus species. Key Aspergillus species which may be targeted by the antibodies described herein include A. fumigatus, A. flavus, A. niger, A. terreus, A. nidulans, and A. oryzae.

It is known that there are a large number of Cryptococcus species. Key Cryptococcus species which may be targeted by the antibodies described herein include C. gattii, C. deuterogattii, C. neoformans var grubii and C. neoformans var neoformans.

As described herein, the antibodies of the invention can detect both morphology specific and morphology-independent epitopes with high specificity. The antibodies described herein may thus bind to e.g. C. albicans with high affinity, as determined by dissociation or association constant, relative to other fungal targets. For example, an antibody of the invention may display a binding affinity for C. albicans with a K_{D} of at least 1000 fold or at least 2000 fold lower than a non-Candida pathogenic fungus such as Aspergillus fumigatus and Cryptococcus neoformans and Pneumocystis jirovecii. As used herein, the term “binding” in the context of the binding of an antibody to a predetermined antigen typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less when determined by for instance surface plasmon resonance (SPR) technology in a BIAcore 3000 instrument using the antigen as the ligand and the antibody as the analyte.

Nevertheless an antibody as described herein may bind to the species closely related to C. albicans e.g. C. dubliniensis, C. tropicalis, C. parapsilosis (clonal complex), C. krusei, C. auris (clonal complex), C. glabrata and C. lusitaniae e.g. for example with an affinity within a 1000-fold of the binding to C. albicans (assessed using EC50).

Further, in some embodiments, the antibodies of the invention may display cross-reactivity between different species, e.g. between C. albicans and S. cerevisiae, A. fumigatus. In some embodiments, the antibodies of the invention may bind to Candida and Aspergillus CWPs.

The term “selective” as used herein may refer to an antibody binding a target antigen, epitope or species with a K_{D} about 1000-, 500-, 200-, 100-, 50-, 10-, or about 5-fold lower than the antibody binding a non-target antigen, epitope or species as measured by e.g. SPR at 25° C.

### Preferred CWPs for Use in the Invention

### Pga31

Pga31 is a novel protein with unknown function which has no homologues in S. cerevisiae. Studies have suggested that Pga31 may be involved in the maintenance of cell wall integrity and cell wall organization by regulating the assembly of chitin (Pitarch, 2008). Plaine et al., (2008) showed that deletion of pga31 in C. albicans led to significantly reduced chitin levels in the cell wall and caspofungin hypersensitivity, suggesting a role for Pga31 in the cell wall salvage mechanism. This protein is 293 amino acids long and has a molecular mass of 29.735 kDa. There are two main glycosylation sites at positions 131 and 190. Furthermore, there is a lipidation at position 271, which suggests linkage to GPI-anchor at that position (Information is based on BLAST and UniProt search).

Pga31 of C. albicans has been detected in the cell wall by performing proteomic analysis on cells grown under certain conditions such as stress, alternative carbon sources, and treatment with cell wall perturbing agents and when cells switch to the opaque form (Castillo et al., 2006, Ene et al., 2012, Pitarch et al., 2008). For example, Pga31 is expressed when cells are grown on lactate as a carbon source (Ene et al., 2012). Without wishing to be bound by any theory, the inventors believe that PGA31 expression may be increased in a murine model of systemic candidiasis compared to growth in vitro.

Proteomic analyses performed by the inventors have shown that Pga31 is specifically detected in the cell wall of C. albicans in response to cell wall stress, e.g. caspofungin treatment and in white opaque switching. Proteomic analyses of signalling mutants suggested that the caspofungin-induced expression of Pga31 in the cell wall was dependent on components of the PKC pathway.

### Utr2

Utr2, is a transglycosidase that is expressed on C. albicans yeast, pseudohyphae and hyphal cell surface, specifically in chitin rich areas. It is a member of the Crh family and has a significant role in cell wall biosynthesis, integrity and maintenance. Studies have demonstrated that deletion of the UTR2 gene results in a decrease in virulence and adhesion to host cells.

It shares structural sequences with two proteins of S. cerevisiae (Utr2 and Crh1) (Pardini et al., 2006). Without wishing to be bound by any theory, the inventors believe that expression of Utr2 is induced by caspofungin as a response to cell wall stress and activation of the calcineurin pathway.

Utr2 has also been found in other fungal pathogens, such as Aspergillus fumigatus which contains conserved amino acid blocks representing the glycosylhydrolase domain (Alberti-Segui et al., 2004) and Cryptococcus neoformans (Farrer et al., 2016; Lee et al., 2016).

### Phr2

Phr2 (pH-reactive protein) is a member of the Gas family of glucanosyltransferases which work by cleaving β-(1,3)-glucan chains and transferring the cleaved chain to another β-(1,3)-glucan polymer. As the name suggests, expression of PHR2 is modulated in relation to the ambient pH, with optimal pH below 5.5.

Candida albicans strains lacking PHR2 are morphologically abnormal in acidic conditions, but normal at an alkaline pH, suggesting its link to Candida cell morphology. It may also be linked to Candida cell development, with null mutant showing altered growth rate.

PHR2 expression is regulated by the Rim101 gene in response to pH (Muhlschlegel & Fonzi 1997). C. albicans Phr2 shares homology to several other fungal cell wall proteins such as Gas1 in Saccharomyces cerevisiae and the Gel family in Aspergillus species (Muhlschlegel & Fonzi 1997).

### Cht2

Cht2 (chitinase 2) is a member of the chitinase family of enzymes that hydrolyse chitin polymers (McCreath et al., 1995-10.1073/pnas.92.7.2544). Cht2 has a glycoside hydrolase catalytic domain and there are homologues in a wide range of fungal species. Cht2 is covalently attached to the fungal cell wall by a modified GPI-anchor. Its gene expression is regulated by Bcr1 the major biofilm transcriptional regulator (Nobile & Mitchell 2006-10.1111/j. 1462-5822.2006.00761.x) and in addition, gene expression is modulated by echinocandin treatment.

### Treatment of Disease

An antibody of the invention may be used for clinical benefit in the treatment of a fungus-associated condition, and particularly infections caused by Candida species, i.e. candidiasis, Aspergillus species, i.e. aspergillosis, and Cryptococcus species, i.e. cryptococcosis. In some embodiments, the antibody may be used to treat infections caused by Mucor species, such as mucormycosis.

The antibodies as described herein may be useful in the surgical and other medical procedures which may lead to immunosuppression, or medical procedures in patients who are already immunosuppressed.

Patients suitable for treatment as described herein include patients with conditions in which fungal infection is a symptom or a side-effect of treatment or which confer an increased risk of fungal infection or patients who are predisposed to or at increased risk of fungal infection, relative to the general population. For example, an antibody as described herein may also be useful in the treatment or prevention of fungal infection in cancer patients. Nevertheless, a subject to be treated may be any animal or human. The subject is preferably mammalian, more preferably human. The subject may be male or female. The subject may be a patient. Therapeutic uses may be in human or animals (veterinary use).

An antibody as described herein may be used in a method of treatment of the human or animal body, including prophylactic or preventative treatment (e.g. treatment before the onset of a condition in an individual to reduce the risk of the condition occurring in the individual; delay its onset; or reduce its severity after onset). The method of treatment may comprise administering the antibody to an individual in need thereof.

Aspects of the invention provide; an antibody as described herein for use in a method of treatment of the human or animal body; an antibody as described herein for use in a method of treatment of a fungal infection; the use of an antibody as described herein in the manufacture of a medicament for the treatment of a fungal infection; and a method of treatment of a fungal infection comprising administering an antibody as described herein to an individual in need thereof. The fungal infection may be candidiasis, aspergillosis, or cryptococcosis.

Also provided is a method for diagnosing a fungal infection in an individual which is caused by fungal species, the method comprising (i) contacting a biological sample obtained from the individual with an antibody described herein; and (ii) determining whether the antibody binds to the biological sample, wherein binding of the antibody to the biological sample indicates the presence of a fungal infection.

Also provided herein is a method for detecting the presence or absence of a fungal species, the method comprising (i) contacting a sample suspected of containing the fungus with an antibody described herein; and (ii) determining whether the antibody binds to the sample, wherein binding of the antibody to the sample indicates the presence of the fungus.

The methods disclosed herein may lead to reduced fungal burden in the subject's brain and/or spleen and/or kidney. The methods may also lead to reduced fungal burden in the subject's lungs and/or liver. In some cases, the antibodies disclosed herein are superior to conventional antifungals due to reduced off-target affects and/or improved half-life and/or specificity. The antibodies may be superior in treating resistant fungal infections than conventional antifungals, for example C. glabrata and C. auris. In some cases, the antibodies may treat echinocandin-resistant C. glabrata and C. auris fungal infections.

The methods disclosed herein may also lead to reduced levels of toxicity, treatment of caspofungin-resistant strains. The antibodies may also be used in co-therapy with e.g. caspofungin, thus reducing caspofungin toxicity through broadening of the therapeutic window. The antibodies may also be used in an ADC by delivering the drug only to the point of need. Such uses may reduce resistance development.

### Pharmaceutical Compositions and Dosage Regimens

Antibodies of the invention may be comprised in pharmaceutical compositions with a pharmaceutically acceptable excipient.

A pharmaceutically acceptable excipient may be a compound or a combination of compounds entering into a pharmaceutical composition which does not provoke secondary reactions and which allows, for example, facilitation of the administration of the antibody, an increase in its lifespan and/or in its efficacy in the body or an increase in its solubility in solution. These pharmaceutically acceptable vehicles are well known and will be adapted by the person skilled in the art as a function of the mode of administration of the antibody.

In some embodiments, antibodies may be provided in a lyophilised form for reconstitution prior to administration. For example, lyophilised antibodies may be re-constituted in sterile water and mixed with saline prior to administration to an individual.

Antibodies will usually be administered in the form of a pharmaceutical composition, which may comprise at least one component in addition to the antibody. Thus pharmaceutical compositions may comprise, in addition to the antibody, a pharmaceutically acceptable excipient, carrier, buffer, stabiliser or other materials well known to those skilled in the art.

Such materials should be non-toxic and should not interfere with the efficacy of the antibody. The precise nature of the carrier or other material will depend on the route of administration, which may be by bolus, infusion, injection or any other suitable route, as discussed below.

For parenteral, for example sub-cutaneous or intra-venous administration, e.g. by injection, the pharmaceutical composition comprising the antibody may be in the form of a parenterally acceptable aqueous solution which is pyrogen-free and has suitable pH, isotonicity and stability. Those of relevant skill in the art are well able to prepare suitable solutions using, for example, isotonic vehicles, such as Sodium Chloride Injection, Ringer's Injection, or Lactated Ringer's Injection. Preservatives, stabilisers, buffers, antioxidants and/or other additives may be employed as required including buffers such as phosphate, citrate and other organic acids; antioxidants, such as ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens, such as methyl or propyl paraben; catechol; resorcinol; cyclohexanol; 3′-pentanol; and m-cresol); low molecular weight polypeptides; proteins, such as serum albumin, gelatin or immunoglobulins; hydrophilic polymers, such as polyvinylpyrrolidone; amino acids, such as glycine, glutamine, asparagines, histidine, arginine, or lysine; monosaccharides, disaccharides and other carbohydrates including glucose, mannose or dextrins; chelating agents, such as EDTA; sugars, such as sucrose, mannitol, trehalose or sorbitol; salt-forming counter-ions, such as sodium; metal complexes (e.g. Zn-protein complexes); and/or non-ionic surfactants, such as TWEEN™, PLURONICS™ or polyethylene glycol (PEG).

Administration is normally in a “therapeutically effective amount”, this being sufficient to show benefit to a patient. Such benefit may be at least amelioration of at least one symptom. The actual amount administered, and rate and time-course of administration, will depend on the nature and severity of what is being treated, the particular mammal being treated, the clinical condition of the individual patient, the cause of the disorder, the site of delivery of the composition, the method of administration, the scheduling of administration and other factors known to medical practitioners. Prescription of treatment, e.g. decisions on dosage etc., is within the responsibility of general practitioners and other medical doctors and may depend on the severity of the symptoms and/or progression of a disease being treated. Specific dosages may be indicated herein or in the Physician's Desk Reference (2003) as appropriate for the type of medicament being administered may be used. A therapeutically effective amount or suitable dose of an antibody may be determined by comparing its in vitro activity and in vivo activity in an animal model. Methods for extrapolation of effective dosages in mice and other test animals to humans are known. The precise dose will depend upon a number of factors, including whether the antibody is for prevention or for treatment, the size and location of the area to be treated, the precise nature of the antibody (e.g. whole antibody, fragment) and the nature of any detectable label or other molecule attached to the antibody.

A typical antibody dose will be in the range 100 μg to 1 g for systemic applications, and 1 μg to 1 mg for topical applications. An initial higher loading dose, followed by one or more lower doses, may be administered.

The antibody may be a whole antibody, e.g. the IgG1 or IgG2a isotype, and where a whole antibody is used, dosages at the lower end of the ranges described herein may be preferred. This is a dose for a single treatment of an adult patient, which may be proportionally adjusted for children and infants, and also adjusted for other antibody formats in proportion to molecular weight. In diagnostic applications, the antibody may be an IgG2a antibody. In therapeutic applications, the antibody may be an IgG1 antibody. In some cases, the antibody may be used in in vivo diagnostic settings, for example in order to image and/or find sources or sites of infection. In some embodiments, the antibody in this and other cases is an IgG4 isotype. The IgG antibody may also be a modified or engineered IgG antibody for further improved antigen binding properties, effector functions, pharmacokinetics, pharmaceutical properties and safety profiles (Igawa et al. (2011)-doi.org/10.4161/mabs.3.3.15234). In some cases, the Fc region of the modified IgG antibody is modified e.g. in order to improve effector function, stability, biodistribution or immunogenicity (Kang et al., (2019)-doi.org/10.1038/s12276-019-0345-9).

Preferably the antibody or fragment will be dosed at no more than 50 mg/kg or no more than 100 mg/kg in a human patient, for example between 1 and 50, e.g. 5 to 40, 10 to 30, 10 to 20 mg/kg.

Treatments may be repeated at daily, twice-weekly, weekly or monthly intervals, at the discretion of the physician. The treatment schedule for an individual may be dependent on the pharmacokinetic and pharmacodynamic properties of the antibody composition, the route of administration and the nature of the condition being treated.

Treatment may be periodic, and the period between administrations may be about two weeks or more, e.g. about three weeks or more, about four weeks or more, about once a month or more, about five weeks or more, or about six weeks or more. For example, treatment may be every two to four weeks or every four to eight weeks. Treatment may be given before, and/or after surgery, and/or may be administered or applied directly at the anatomical site of surgical treatment or invasive procedure. Suitable formulations and routes of administration are described above.

In some embodiments, antibodies as described herein may be administered as sub-cutaneous injections. Sub-cutaneous injections may be administered using an auto-injector, for example for long term prophylaxis/treatment.

In some preferred embodiments, the therapeutic effect of the antibody may persist for several half-lives, depending on the dose. For example, the therapeutic effect of a single dose of antibody may persist in an individual for 1 month or more, 2 months or more, 3 months or more, 4 months or more, 5 months or more, or 6 months or more.

It will be understood that the term “treatment” as used herein includes combination treatments and therapies, in which two or more treatments, therapies, or agents are combined, for example, sequentially or simultaneously.

The agents (i.e. the antibodies described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g. 1, 2, 3, 4 or more hours apart, or even longer periods apart where required), the precise dosage regimen being commensurate with the properties of the therapeutic agent(s) as described herein, including their synergistic effect.

In some embodiments, the antibodies are effective when administered before infection. In some embodiments, the antibodies are effective when administered before infection, without administration after infection. They may be administered 1, 2, 3, 4, 5, 6, 12 or 24 hours before infection. They may be administered 1, 2, 3, 4, 5, 6 or 7 days before infection. They may be administered 1, 2, 3 or 4 weeks before infection. They may be administered 1, 2, 3, 4, 5, 6, 12, 24, 36 or 48 months before infection.

In some embodiments, the antibodies are effective when administered after infection. In some embodiments, the antibodies are effective when administered after infection, without administration before infection. In some embodiments, the antibodies are effective when administered two, three, four or more times after infection. In some embodiments, the antibodies are effective when administered only once after infection. They may be administered 1, 2, 3, 4, 5 or 7 days after infection. They may be administered 1, 2, 3 or 4 weeks after infection. They may be administered 1, 2, 3, 6 or 12 months after infection. The antibodies disclosed herein may require lower dosing than for conventional antifungal agents, thereby reducing resistance development. For example, the antibodies may require daily dosing for less than 30, 25, 20, 18, 16, 14, 12, 10, 9, 8, 7, 6, 5, 4 or 3 days.

The agents (i.e. the antibodies described here, plus one or more other agents) may be formulated together in a single dosage form, or alternatively, the individual agents may be formulated separately and presented together in the form of a kit, optionally with instructions for their use.

For example, the compounds described herein may in any aspect and embodiment also be used in combination therapies, e.g. in conjunction with other agents e.g. antifungal agents. The second antifungal agent may be selected from an azole (e.g. fluconazole), a polyene (e.g. amphotericin B), an echinocandin (e.g. caspofungin), an allylamine (e.g. terbinafine), and a flucytosine (also called 5-fluorocytosine). The skilled person will recognise that other antifungal agents may also be used. In some embodiments, the second antifungal agent is a second antifungal antibody or an antimicrobial peptide. In some embodiments, the antibody described herein is conjugated to the second antifungal agent.

The antibodies described herein may also be utilised to isolate and identify protective antigens for development as fungal vaccines, or prepare or identify other therapeutic moieties. The invention also provides a composition of matter comprising a pharmaceutical composition described herein, and a further antifungal agent.

### Detection and Diagnosis

Antibodies as described herein may also be useful in in vitro testing, for example in the detection or labelling of fungus or a fungal infection, for example in a sample obtained from a patient.

Antibodies as described herein may be useful for identifying C. albicans, and/or distinguishing C. albicans from other fungi. Antibodies as described herein may be useful for identifying A. fumigatus, and/or distinguishing A. fumigatus from other fungi. Antibodies as described herein may also be useful for distinguishing a fungal infection from a bacterial and/or viral infection.

The presence or absence of a fungus (e.g. C. albicans or A. fumigatus) may be detected by
(i) contacting a sample suspected of containing the fungus with an antibody described herein, and
(ii) determining whether the antibody binds to the sample, wherein binding of the antibody to the sample indicates the presence of the fungus.

A fungal infection, e.g. candidiasis, aspergillosis, or cryptococcosis infection, in an individual may be diagnosed by
(i) obtaining a sample from the individual;
(ii) contacting the sample with an antibody as described herein, and
(iii) determining whether the antibody binds to the sample, wherein binding of the antibody to the sample indicates the presence of the fungal infection.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container.

Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle).

At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to an antibody variant.

### Binding Assays and Other Assays

In one aspect, an antibody of the invention is tested for its antigen binding activity, e.g., by known methods such as ELISA, Western blot, etc.

In an exemplary competition assay, immobilized antigen is incubated in a solution comprising a first labeled antibody that binds to the antigen and a second unlabeled antibody that is being tested for its ability to compete with the first antibody for binding to the antigen. The second antibody may be present in a hybridoma supernatant. As a control, immobilized antigen is incubated in a solution comprising the first labeled antibody but not the second unlabeled antibody. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antibody is competing with the first antibody for binding to the antigen (See Harlow, and Lane (1988) Antibodies: A Laboratory Manual ch. 14 (Cold Spring Harbor Laboratory, Cold Spring Harbor, NY).

Binding of antibodies to a sample may also be determined using any of a variety of techniques known in the art, for example ELISA, immunocytochemistry, immunoprecipitation, affinity chromatography, and biochemical or cell-based assays. In some embodiments, the antibody is conjugated to a detectable label or a radioisotope.

### Sequence Identity

Percentage (%) sequence identity is defined as the percentage of amino acid residues in a candidate sequence that are identical with residues in the given listed sequence (referred to by the SEQ ID NO) after aligning the sequences and introducing gaps if necessary, to achieve the maximum sequence identity, and not considering any conservative substitutions as part of the sequence identity. Sequence identity is preferably calculated over the entire length of the respective sequences.

Where the aligned sequences are of different length, sequence identity of the shorter comparison sequence may be determined over the entire length of the longer given sequence or, where the comparison sequence is longer than the given sequence, sequence identity of the comparison sequence may be determined over the entire length of the shorter given sequence.

Alignment for purposes of determining percent amino acid sequence identity can be achieved in various ways known to a person of skill in the art, for instance, using publicly available computer software such as ClustalW 1.82. T-coffee or Megalign (DNASTAR) software. When using such software, the default parameters, e.g. for gap penalty and extension penalty, are preferably used. The default parameters of ClustalW 1.82 are: Protein Gap Open Penalty=10.0, Protein Gap Extension Penalty=0.2, Protein matrix=Gonnet, Protein/DNA ENDGAP=−1, Protein/DNA GAPDIST=4.

### Selected Definitions

The term “antibody” (Ab) in the context of the present invention refers to an immunoglobulin molecule, a fragment of an immunoglobulin molecule, or a derivative of either thereof, which has the ability to specifically bind to an antigen under typical physiological conditions. The variable regions of the heavy and light chains of the immunoglobulin molecule contain a binding domain that interacts with an antigen. The constant regions of the antibodies (Abs) may mediate the binding of the immunoglobulin to host tissues or factors, including various cells of the immune system (such as effector cells) and components of the complement system such as C1q, the first component in the classical pathway of complement activation.

The term “antibody”, unless specified otherwise, also includes polyclonal antibodies, monoclonal antibodies (mAbs), antibody-like polypeptides, such as chimeric antibodies, and humanized antibodies, bispecific antibodies, and antibody fragments retaining the ability to specifically bind to the antigen (antigen-binding fragments) provided by any known technique, such as enzymatic cleavage, peptide synthesis, and recombinant techniques (see e.g. doi.org/10.1007/978-1-59745-198-7_203). The term “antibody” may also include VHH, VH or VNAR single domain antibodies. The constant region domains, in particular in the Fc domain, where present, are may be of IgG2a isotype. Accordingly, each heavy chain may comprise an IgG2a CH2 domain and a CH3 domain. The constant region domains may also be of the human IgG1, IgG3 or IgG4 isotype. The constant region domain may also be engineered.

As used herein, the term “binding” in the context of the binding of an antibody to a predetermined antigen typically is a binding with an affinity corresponding to a K_{D} of about 10⁻⁷ M or less, such as about 10⁻⁸ M or less, such as about 10⁻⁹ M or less, about 10⁻¹⁰ M or less, or about 10⁻¹¹ M or even less when determined by for instance surface plasmon resonance (SPR) technology in a BIAcore 3000 instrument using the antigen as the ligand and the antibody as the analyte.

The term “chimeric antibody” refers to an antibody that contains one or more regions from one antibody and one or more regions from one or more other antibodies. The term “chimeric antibody” includes divalent and polyvalent antibodies. Chimeric antibodies are produced by recombinant processes well known in the art (see for instance Cabilly et al., PNAS USA 81, 3273-3277 (1984), Morrison et al., PNAS USA 81, 6851-6855 (1984), Boulianne et al., Nature 312, 643-646 (1984), EP125023, Neuberger et al., Nature 314, 268-270 (1985), Sahagan et al., J. Immunol. 137, 1066-1074 (1986), Liu et al., PNAS USA 84, 3439-3443 (1987), Sun et al., PNAS USA 84, 214-218 (1987), Better et al., Science 240, 1041-1043 (1988) and Harlow et al., Antibodies: A Laboratory Manual, Cold Spring Harbor Laboratory Press, Cold Spring Harbor, N.Y., (1988)). Variable domains of rodent origin may be fused to constant domains of human origin such that the resultant antibody retains the antigenic specificity of the rodent parented antibody, and vice versa. Antibodies according to the present invention may be chimeric antibodies.

That antigenic specificity is conferred by variable domains and is independent of the constant domains is known from experiments involving the bacterial expression of antibody fragments, all containing one or more variable domains. These molecules include Fab-like molecules (Better et al. (1988) Science 240, 1041); Fv molecules (Skerra et al. (1988) Science 240, 1038); single-chain Fv (ScFv) molecules where the VH and VL partner domains are linked via a flexible oligopeptide (Bird et al. (1988) Science 242, 423; Huston et al. (1988) Proc. Natl. Acad. Sd. USA 85, 5879) and single domain antibodies (dAbs), such as VHH, VH or VNAR antibodies, comprising isolated V domains (see e.g. Ward et al. (1989) Nature 341, 544 and Wesolowski et al. (2009) Med Microbiol Immunol. 198 (3): 157-174). A general review of the techniques involved in the synthesis of antibody fragments which retain their specific binding sites is to be found in Winter & Milstein (1991) Nature 349, 293-299.

The term “human antibody”, as used herein, is intended to include antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences. Antibodies according to the present invention may be human antibodies.

The term “immunoglobulin” refers to a class of structurally related glycoproteins consisting of two pairs of polypeptide chains, one pair of light (L) low molecular weight chains and one pair of heavy (H) chains, all four inter-connected by disulfide bonds. The structure of immunoglobulins has been well characterized. See for instance Fundamental Immunology Ch. 7 (Paul, W., ed., 2nd ed. Raven Press, N.Y. (1989)). Briefly, each heavy chain typically is comprised of a heavy chain variable region (abbreviated herein as VH or VH) and a heavy chain constant region. The heavy chain constant region typically is comprised of three domains, CH1, CH2, and CH3. Each light chain typically is comprised of a light chain variable region (abbreviated herein as VL or VL) and a light chain constant region. The light chain constant region typically is comprised of one domain, CL or CL. The VH and VL regions may be further subdivided into regions of hypervariability (or hypervariable regions which may be hypervariable in sequence and/or form of structurally defined loops), also termed complementarity determining regions (CDRs), interspersed with regions that are more conserved, termed framework regions (FRs). Each VH and VL is typically composed of three CDRs and four FRs, arranged from amino-terminus to carboxy-terminus in the following order: FR1, CDR1, FR2, CDR2, FR3, CDR3, FR4 (see also Chothia and Lesk J. Mol. Biol. 196, 901-917 (1987)).

Often, the numbering of amino acid residues is performed by the method described in Kabat et al., Sequences of Proteins of Immunological Interest, 5th Ed. Public Health Service, National Institutes of Health, Bethesda, Md. (1991). Using this numbering system, the actual linear amino acid sequence of a peptide may contain fewer or additional amino acids corresponding to a shortening of, or insertion into, a FR or CDR of the variable domain. For example, a heavy chain variable domain may include a single amino acid insert (residue 52a according to Kabat) after residue 52 of VH CDR2 and inserted residues (for instance residues 82a, 82b, and 82c, etc. according to Kabat) after heavy chain FR residue 82. The Kabat numbering of residues may be determined for a given antibody by alignment at regions of homology of the sequence of the antibody with a “standard” Kabat numbered sequence.

Alternatively, the numbering of amino acid residues is performed by the EU-index also described in Kabat et al., Sequences of Proteins of Immunological Interest, 5th Ed. Public Health Service, National Institutes of Health, Bethesda, Md. (1991).

An “isolated antibody” as used herein, is intended to refer to an antibody which is substantially free of other antibodies having different antigenic specificities. An isolated antibody that specifically binds to an epitope, isoform or variant of a particular human target antigen may, however, have cross-reactivity to other related antigens, for instance from other species (such as species homologs). Moreover, an isolated antibody may be substantially free of other cellular material and/or chemicals. Antibodies according to the present invention may be provided in isolated form.

The term “monoclonal antibody” or “mAb” as used herein refers to an antibody from a population of substantially homogeneous antibodies, i.e., all of the individual antibodies comprising the population are identical and/or bind the same epitope as each other, except for possible product-related impurities such as variant antibodies, e.g., antibodies containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is typically directed against the same determinant (or determinants, in the case of multispecific monoclonal antibodies) on an antigen as each other. Thus, the modifier “monoclonal” indicates the character of the antibody as being obtained from a substantially homogeneous population of antibodies, and is not to be construed as requiring production of the antibody by any particular method. The term “mAb” as used herein includes antibodies, antibody fragments and antibody fusion proteins. A mAb may be monospecific or multispecific (e.g. bispecific). Monoclonal antibodies are comprised of different polypeptide chains. A regular IgG antibody comprises two identical heavy chains and two identical light chains. More complex antibodies, particularly multispecific antibodies, usually comprise more than two different polypeptides, which results in the issue of possible mispairings or incompleteness upon recombinant expression. Antibodies according to the present invention may preferably be monoclonal antibodies. Suitable monoclonal antibodies can be prepared using methods well known in the art (e.g. see Köhler, G.; Milstein, C. (1975). “Continuous cultures of fused cells secreting antibody of predefined specificity”. Nature 256 (5517): 495; Siegel D L (2002). “Recombinant monoclonal antibody technology”. Schmitz U, Versmold A, Kaufmann P, Frank H G (2000); “Phage display: a molecular tool for the generation of antibodies—a review”. Placenta. 21 Suppl A: S106-12. Helen E. Chadd and Steven M. Chamow; “Therapeutic antibody expression technology,” Current Opinion in Biotechnology 12, no. 2 (Apr. 1, 2001): 188-194; McCafferty, J.; Griffiths, A.; Winter, G.; Chiswell, D. (1990). “Phage antibodies: filamentous phage displaying antibody variable domains”. Nature 348 (6301): 552-554; “Monoclonal Antibodies: A manual of techniques” H Zola (CRC Press, 1988) and in “Monoclonal Hybridoma Antibodies: Techniques and Applications”, J G R Hurrell (CRC Press, 1982). Chimaeric antibodies are discussed by Neuberger et al. (1988, 8th International Biotechnology Symposium Part 2, 792-799)).

As used herein the term “resistant”, in the context of an antifungal agent's effect on a pathogen, may be defined as meaning that the MIC of the agent against the pathogen is higher than the breakpoint for a given organism, where it is clinically defined. As used herein the term “susceptible”, in the context of an antifungal agent's effect on a pathogen, may be defined as meaning that the MIC of the agent against the pathogen is lower than the breakpoint for a given organism. As used herein the term “intermediate” (or “susceptible, increased exposure”—Arendrup (2020) doi.org/10.1016/j.cmi.2020.06.007), in the context of an antifungal agent's effect on a pathogen, may be defined as meaning that the agent may be effective against a pathogen at high doses or if the agent concentrates at a tissue site.

The breakpoint is the concentration that the pathogen is susceptible to the antimicrobial agent.

Within a single species there will be examples of strains that fall into the three classes defined above. Drug resistance may be acquired (due to mutations induced by exposure to drug) or intrinsic.

MIC and breakpoints for a given antifungal agent and pathogen may be readily determined (see e.g. eucast.org/astoffungi/clincalbreakpointsforantifungals)

As used herein, a “surface-exposed CWP” is a CWP, or fragment thereof, that is wholly or partially exposed to the external side of a cell wall.

The invention includes the combination of the aspects and preferred features described except where such a combination is clearly impermissible or expressly avoided. Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

Any sub-titles herein are included for convenience only, and are not to be construed as limiting the disclosure in any way. The invention will now be further described with reference to the following non-limiting Figures and Examples. Other embodiments of the invention will occur to those skilled in the art in the light of these. The disclosure of all references cited herein, inasmuch as it may be used by those skilled in the art to carry out the invention, is hereby specifically incorporated herein by cross-reference.

### FIGURES

FIG. 1

An exemplary schematic representation of the steps involved in the cell wall proteome study of various pathogenic fungal species and identification of surface exposed protein epitopes which can be utilised as potential antigens for antibody development.

FIG. 2

Expression levels of cell wall proteins Pga31, Utr2, and Phr2 from various clinical isolates of C. albicans resistant and susceptible to the echinocandin drug caspofungin. Strains were grown in the presence or absence of caspofungin (CAS) and differences in the levels of proteins expressed were detected by LC-MS/MS. (A-C) Levels of Pga31, Utr2 and Phr2 proteins in C. albicans caspofungin susceptible and resistant strains grown without the drug (SC5314 and K063-3 respectively) and with MIC of the drug (SC5314+CAS and K063-3+CAS respectively). (D-E) Pga31 expression levels of C. albicans caspofungin susceptible strains and resistant strains grown in the presence or absence of the drug. (F-G) Utr2 expression levels of C. albicans caspofungin susceptible strains and resistant strains grown in the presence or absence of the drug. (H-I) Phr2 expression levels of C. albicans caspofungin susceptible strains and resistant strains grown in the presence or absence of the drug.

FIG. 3

Proteomic analysis of cell wall fractions from C. tropicalis clinical isolates grown at 37° C. overnight in RPMI-1640 medium in the absence (A) or presence (B) of caspofungin. C. tropicalis clinical isolate Ct1 is drug sensitive and Ct2 is drug resistant. In the Venn diagrams, the total number of proteins with at least 2 peptides identified by LC MS/MS is shown. The analysis was performed with Proteome discoverer 2.2 software and a 2 peptides per protein cut off was applied. (C) Table describing changes in the wall proteome of C. tropicalis isolates Ct1 and Ct2 induced with caspofungin, indicated by the ratios of the peak areas in the chromatogram and the number of peptide-spectrum matches (#PSM). Proteins selected for antibody generation are highlighted in grey.

FIG. 4

Antigen binding ELISA of Pga31 clones. Two-fold serial dilutions of Pga31 scAbs were performed and checked for peptide antigen binding where the ELISA plate was coated with Pga31 peptide-biotin conjugate. ScAb binding was detected using 1/1000 dilution of anti-human kappa light chain-HRP conjugated antibody.

FIG. 5

Pga31 scAbs binding to the total cell lysates of WT C. albicans (SC5314) treated with or without 0.032 μg/ml caspofungin. (A) Pga31 scAb 1B11 binding (B) Pga31 scAb 1G4 binding. Doubling dilutions of scAbs were added to the plates coated with WT C. albicans (−/+caspofungin) and detected using anti-human C kappa HRP conjugated secondary antibody.

FIG. 6

Pga31 scAbs binding to the cell lysates of pga314 mutant strain treated with or without 0.032 μg/ml caspofungin. (A) Pga31 scAb 1B11 binding (B) Pga31 scAb 1G4 binding. Doubling dilutions of scAbs were added to the plates coated with pga314 mutant (−/+caspofungin) and detected using anti-human C kappa HRP conjugated secondary antibody.

FIG. 7

Testing 1B11 scAb activity under biofilm-inducing conditions. (A, B) Measurement of C. auris and C. albicans biofilm biomass induced with RPMI medium or 20% FC serum with crystal violet. For C. auris 1716, the results are significantly different compared to the control in both RPMI and 20% FCS conditions (T-test, p<0.0001; p<0.0004). Results for C. albicans SC5314 are significantly different in RPMI conditions (T test, p<0.0001). (C, D) Images of the biofilm culture taken after 24 h incubation for 1716 and SC5314 respectively. Data represent the mean±SD of experiments performed on 3 separate occasions, using 3 replicates.

FIG. 8

Assessing the protective effect of 1B11 scAb against Candida in Galleria infection model. (A) Survival curve of larvae infected with C. auris (1716) and treated with 1B11 scAb. The curve demonstrates a significant survival percentage for antibody treated group (Mantel-Cox test, p<0.0001). (B) Survival curve of larvae infected with C. albicans (SC5314) and treated with 1B11 scAb or caspofungin. The curve demonstrates a significant survival percentage in treatment groups (Mantel-Cox test, p<0.0001).

FIG. 9

Fungal burden measurements to assess early antifungal activity of 1B11 scAb (A) C. auris colony formation was inhibited by the antibody and the results are significantly different at 4 h and 24 h (T-test, p<0.079). (B) Fungal growth measured by colony forming units after 4 h and 24 h incubation for larvae infected with SC5314 strain (T-test, p<0.079).

FIG. 10

ELISA based characterisation of 1B11 mAb binding. (A) 1B11 mAb binding to streptavidin captured biotinylated Pga31 peptide (B) assessing the cross-reactivity towards other peptide antigens (C) binding to C. albicans yeast (D) binding to C. albicans hyphae and (E) binding towards Pga31 over-expressing strain of C. albicans. Values represent mean absorbance readings at 450 nm (n=2).

FIG. 11

Fluorescent microscopy of mAb 1B11 binding to C. albicans hyphae. Yeast cells were left for 30 minutes to adhere to glass slide. Cells were then grown in DMEM+10% FCS for approximately 3 h to induce hyphal growth. A fluorescently conjugated anti-mouse antibody was used to detect 1B11 mAb binding (as shown by white arrows, which is away from the yeast head and more localised on the surface of growing hyphae).

FIG. 12

Hyphal length of intracellular C. albicans at 60 minutes and extracellular hyphae at 90 minutes. Intracellular hyphae were measured (μm) following yeast cell uptake by J774.1 mouse macrophage (left). Kruskal-Wallis test with Dunn's Multiple Comparison Test: P=<0.0001. SC5314 vs 1B11 difference in rank sum 60.99. Extracellular hyphae were measured in the same sample that were not engulfed by macrophages (right).

FIG. 13

Study plan for Mouse model of disseminated Candidiasis-Study 1 (1B11 mAb)

FIG. 14

Survival in treatment and control groups 4 days post-infection in Study 1. Caspofungin (1 mg/kg body weight post-infection at 24 h), Saline, Control IgG (15 mg/kg IP) and mAb 1B11 (15 mg/kg IP) administered 3 h pre and 24 h post-infection.

FIG. 15

Mean fungal burdens in kidneys on day 4 post-infection in Study 1. Graph shows mean kidney burden counts in various treatment and control groups. Caspofungin (1 mg/kg body weight post-infection at 24 h), Saline, Control IgG (15 mg/kg IP) and mAb 1B11 (15 mg/kg IP) were administered 3 h pre and 24 h post-infection. Error bars denote standard deviation.

FIG. 16

Study plan for Mouse model of disseminated Candidiasis-Study 2 (1B11 mAb)

FIG. 17

Mice survival in treatment and control groups 6 days post-infection in Study 2. Caspofungin (1 mg/kg body weight post-infection at 24 h and 72 h), saline, Control IgG (12.5 mg/kg IP) and mAb 1B11 (12.5 mg/kg IP) were administered 3 h pre and 24 h and 72 h post-infection.

FIG. 18

Mean fungal burdens in organs 6 days post-infection in Study 2. Kidney, spleen and brain burden counts in various treatment and control groups. Caspofungin (1 mg/kg body weight post-infection at 24 h and 72 h), Saline, Control IgG (12.5 mg/kg IP) and mAb 1B11 (12.5 mg/kg IP) administered 3 h pre and 24 h and 72 h post-infection.

FIG. 19

Study plan for Mouse model of disseminated Candidiasis-Study 3 (1B11 mAb)

FIG. 20

Kidney fungal burdens at day 7 post-infection. Graph shows results where zero cfus have been assigned a value one half log below the detection limit. The horizontal line shows the detection limit for the kidney organ fungal burden assay. Treatment group 1: pre- and post-infection 1B11, 2: post-infection 1B11, 3: saline, 4: caspofungin (1 mg/kg body weight post-infection at 24 h and 72 h post-infection).

FIG. 21

ELISA data showing Utr2 scAbs binding in multiple experiments. (A-C) immobilised Utr2 peptide, (D) C. albicans (SC5314) yeast cell lysate, (E-F) WT C. albicans yeast, (G-H) Utr2 over-expressing strain, (I-J) short and extended hyphae. Short hyphae are defined as yeast cells grown in DMEM+10% FCS at 37° C. for 2 h with an average length of 3 μm. Extended hyphae were grown for 6 h under the same conditions with an average size of 12 μm. Values represent means (n=2).

FIG. 22

Utr2 scAbs binding to the total cell lysates of WT C. albicans (SC5314) treated with or without 0.032 μg/ml caspofungin. (A-F) scAbs A7, B1, C5, C8, C9 and C10 respectively. Doubling dilutions of scAbs were added to the plates coated with WT C. albicans (−/+caspofungin) and detected using anti-human C kappa HRP conjugated secondary antibody.

FIG. 23

Utr2 scAbs binding to the cell lysates of utr2Δ mutant strain treated with or without 0.032 μg/ml caspofungin. (A-G) scAbs A7, B1, C5, C8, C9, C10 and 1H3 respectively. Doubling dilutions of scAbs were added to the plates coated with utr2Δ mutant (−/+caspofungin) and detected using anti-human C kappa HRP conjugated secondary antibody.

FIG. 24

Utr2 scAbs binding to the cell lysates of C. albicans triple mutant strain (utr2Δ: crh11Δ: crh12Δ) treated with or without 0.032 μg/ml caspofungin. (A-G) scAbs A7, B1, C5, C8, C9, C10 and 1H3 respectively. Doubling dilutions of scAbs were added to the plates coated with utr2Δ mutant (−/+caspofungin) and detected using anti-human C kappa HRP conjugated secondary antibody.

FIG. 25

ELISA data showing single-chain antibodies (scAb) binding to Candida albicans (A) and Aspergillus fumigatus hyphae (B). C. albicans yeast cells were grown in DMEM+10% FCS for 2 h at 37° C. to induce hyphal growth. A. fumigatus conidia were grown in RPMI+10% FCS at 37° C. overnight to ensure swelling and germination of conidia. Values represent means (n=2).

FIG. 26

Minimum inhibitory concentration (MIC) analysis showing antifungal activity of scAbs. Experiments showing antifungal activity of antibodies and caspofungin (positive control) against wild-type C. albicans (SC5314) at 24 h (A) and 48 h (B). Overnight cultures were diluted to 2×10⁶ cells/ml in RPMI-1640 before adding 50 μl to each well (˜100,000 cells per well). A starting concentration of 100 μg/ml per antibody or caspofungin was added to designated wells and double diluted across the plate. Values represent means (n=2).

FIG. 27

Immunofluorescence staining of C. albicans WT and Utr2 overexpression strains with 1B1 and 1D2 scAbs. Calcofluor white (CFW) was used to outline the cell wall and a fluorescently conjugated goat anti-human antibody was used to detect scAb binding. The images were viewed using UltraVIEW Vox 3D live cell imaging system (Volocity software). (A-B) 1B1 scAb staining of the cell wall of WT (A) and overexpression strains (B). (C-D) 1D2 scAb staining of the hyphal tips and bud surface (white arrows) of WT (C) and overexpression strains (D). (E-F) repeated immunofluorescence staining with higher concentrations of 1D2 scAb and secondary antibodies to confirm staining to the hyphae (white arrow), especially tips of the hyphae and bud surface of C. albicans WT (E) and overexpression strains (F).

FIG. 28

Study plan for Mouse model of disseminated Candidiasis-Study 1 (ID2)

FIG. 29

Survival in treatment and control groups 4 days post-infection in Study 1 (ID2). Caspofungin (1 mg/kg body weight post-infection at 24 h), Saline, Control IgG (15 mg/kg IP), test mAb 1D2 (15 mg/kg IP) were administered 3 h pre and 24 h post-infection.

FIG. 30

Mean fungal burdens in kidneys on day 4 post-infection in Study 1 (ID2). Graph shows mean kidney burden counts in various treatment and control groups. Caspofungin (1 mg/kg body weight post-infection at 24 h), Saline, Control IgG (15 mg/kg IP), test mAb 1D2 (15 mg/kg IP) were administered 3 h pre and 24 h post-infection.

FIG. 31

Study plan for Mouse model of disseminated Candidiasis-Study 2 (ID2)

FIG. 32

Mice survival in treatment and control groups 6 days post-infection in Study 2 (ID2). Caspofungin (1 mg/kg body weight post-infection at 24 h and 72 h), saline, Control IgG (12.5 mg/kg IP) and mAb 1D2 (12.5 mg/kg IP) were administered 3 h pre and 24 h and 72 h post-infection.

FIG. 33

Mean fungal burdens in organs 6 days post-infection in Study 2 (ID2). Kidney, spleen and brain burden counts in various treatment and control groups. Caspofungin (1 mg/kg body weight post-infection at 24 h and 72 h), Saline, Control IgG (12.5 mg/kg IP) and mAb 1D2 (12.5 mg/kg IP) administered 3 h pre and 24 h and 72 h post-infection.

FIG. 34

Study plan for Mouse model of disseminated Candidiasis (1H3)—Study 3

FIG. 35

Kidney fungal burdens at day 7 post-infection for study 3 (1H3). Graph shows results where zero cfus have been assigned a value one half log below the detection limit. The horizontal line shows the detection limit for the kidney organ fungal burden assay. Treatment group 1: pre- and post-infection 1H3, 2: post-infection 1H3, 3: saline, 4: caspofungin (1 mg/kg body weight post-infection at 24 h and 72 h post-infection).

FIG. 36

ELISA data showing Phr2 scAbs binding. (A) Specific binding of scAb 3C5 to immobilised Phr2 peptide (B) Specific binding of scAb 3D7 to immobilised Phr2 peptide. No cross reactivity was observed to other CWP peptides (C) scAbs binding to wildtype C. albicans (SC5314) yeast.

### EXAMPLES

### Example 1—Methods of Identifying Surface-Exposed Cell Wall Proteins for the Development of Antifungal Antibodies

This aspect of the invention was based, in part, on studying variations in the cell wall proteome of drug resistant Candida isolates and identification of surface exposed protein epitopes.

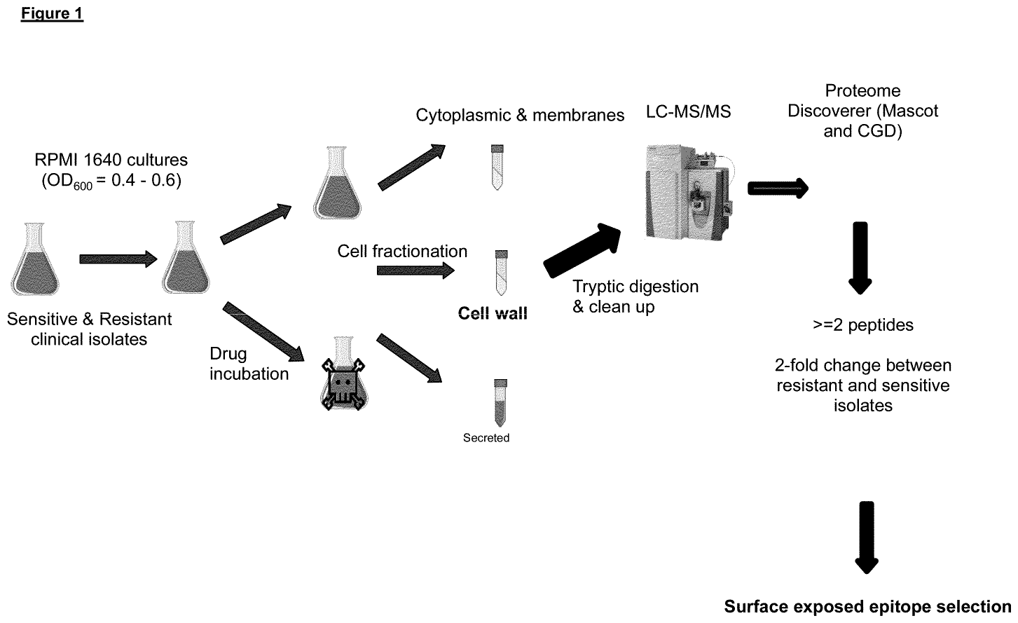

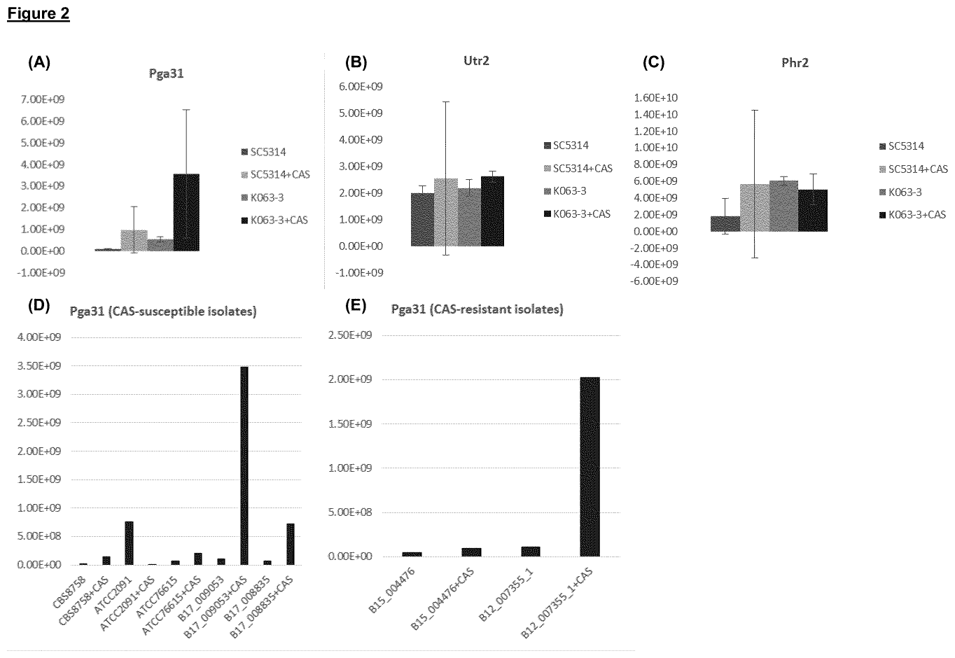

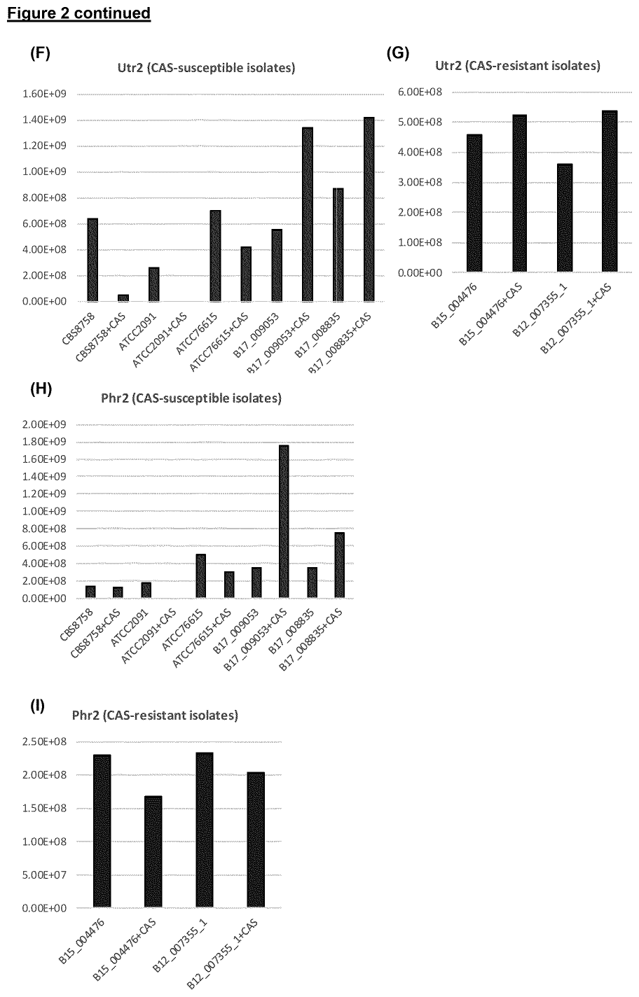

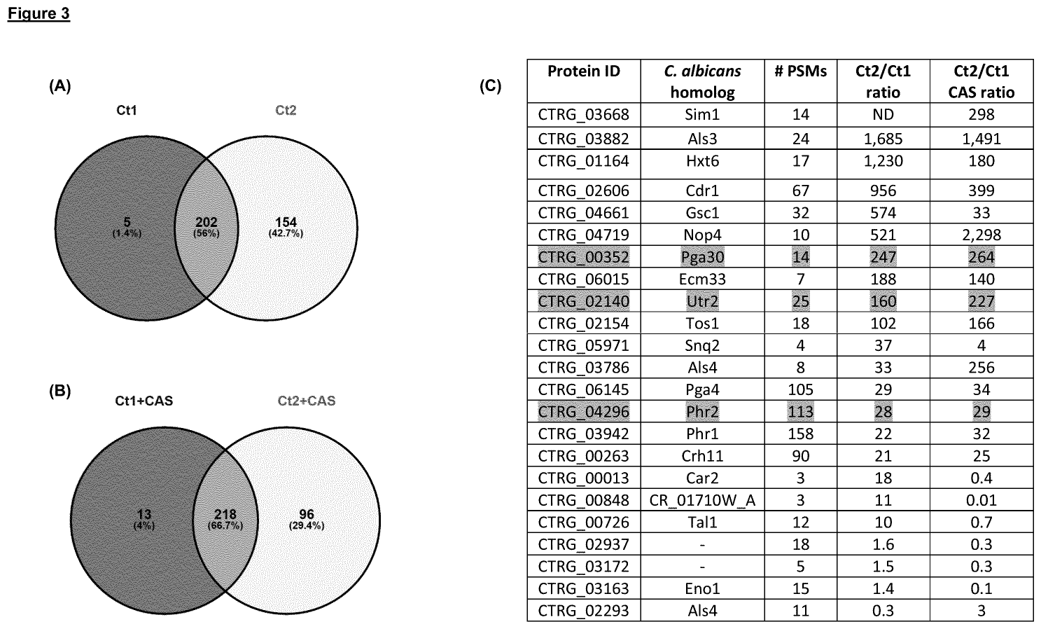

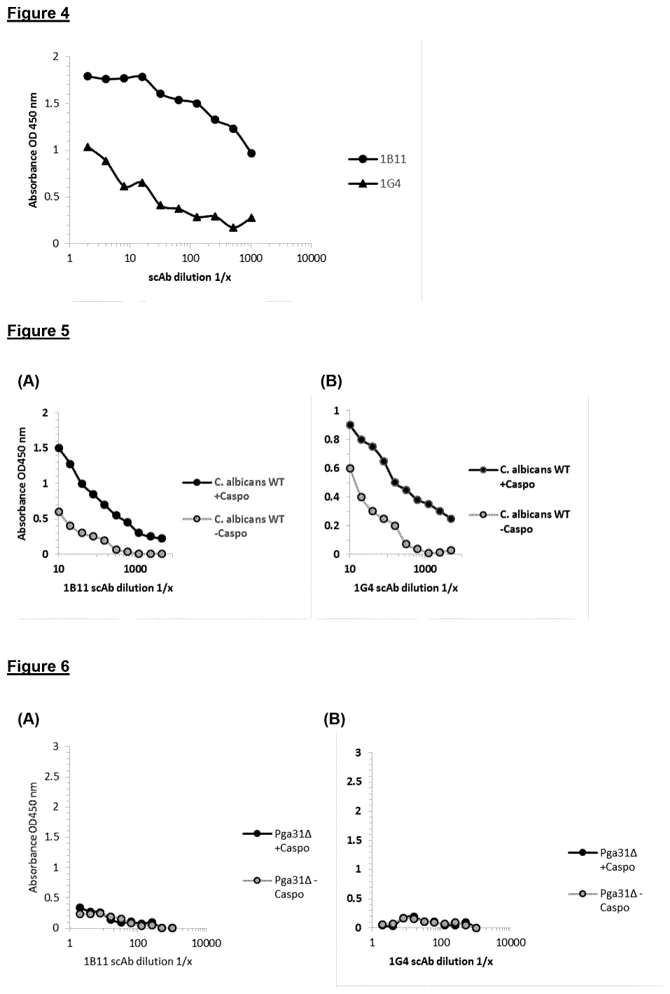

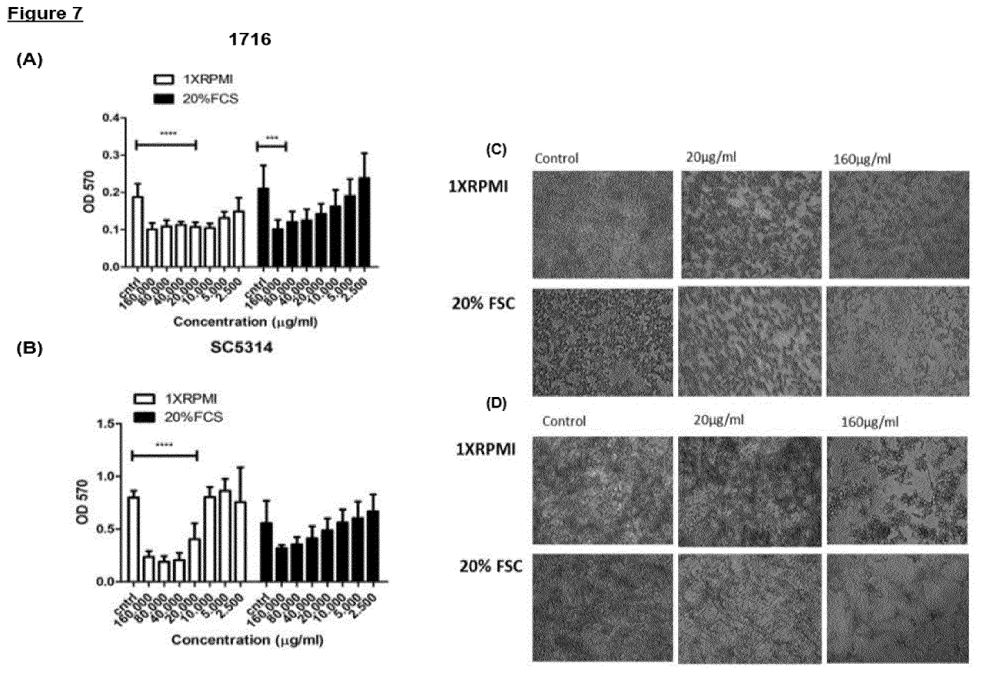

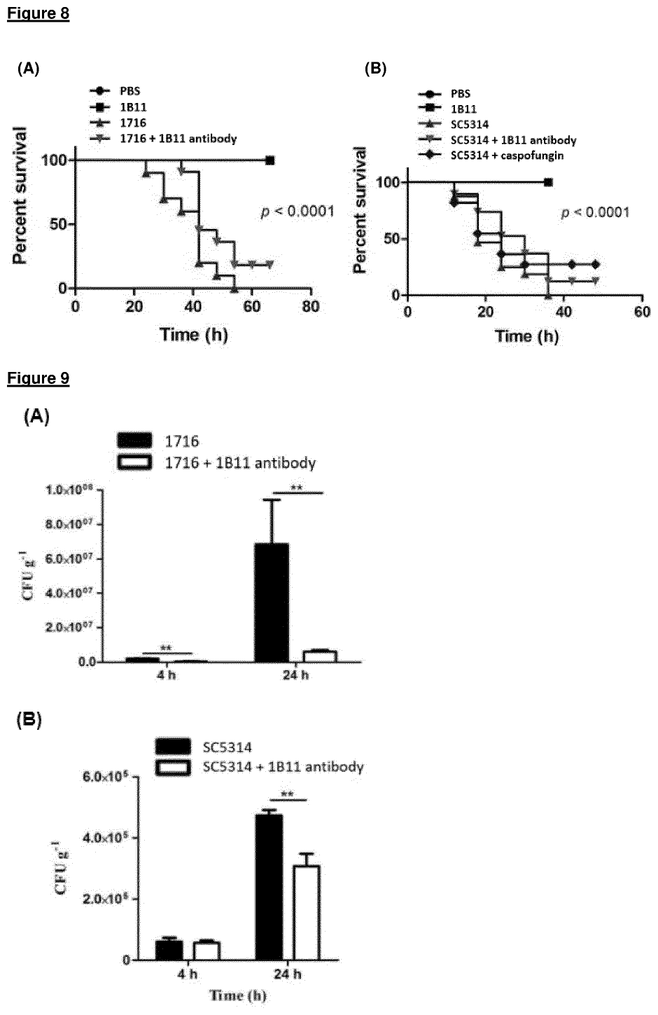

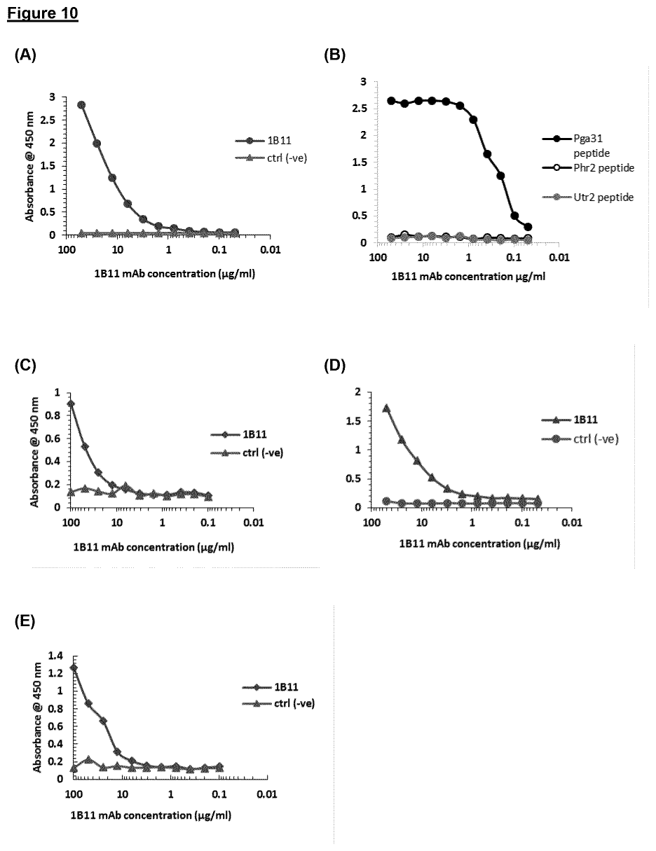

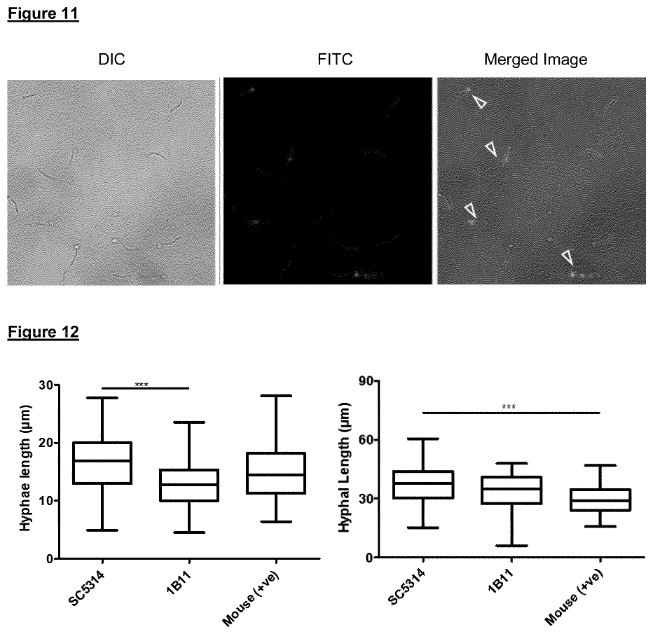

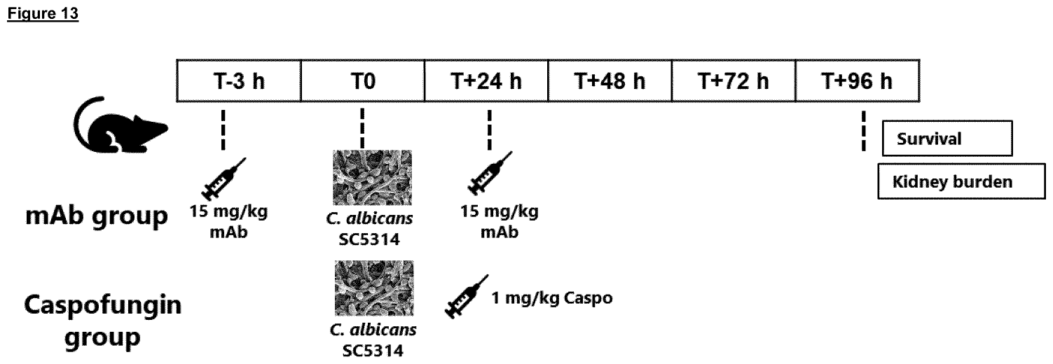

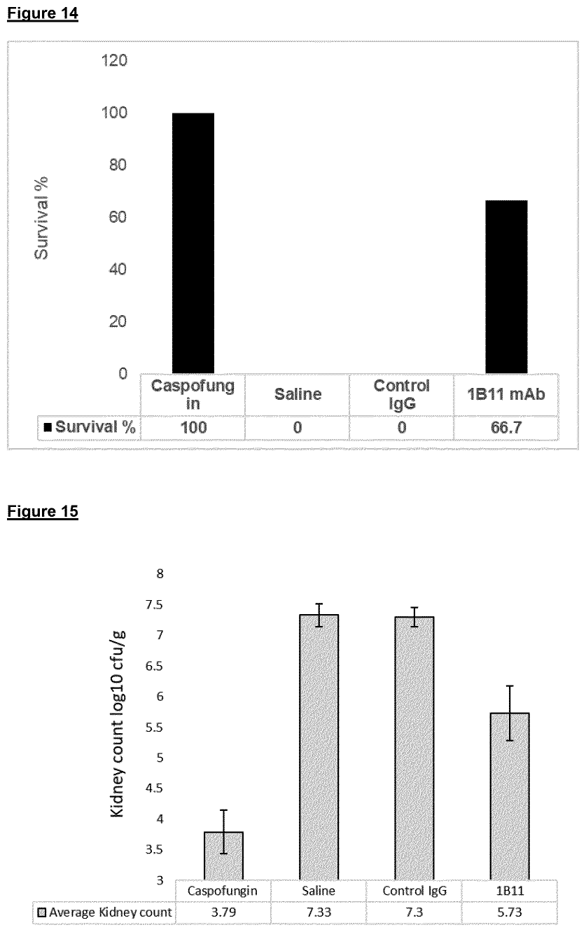

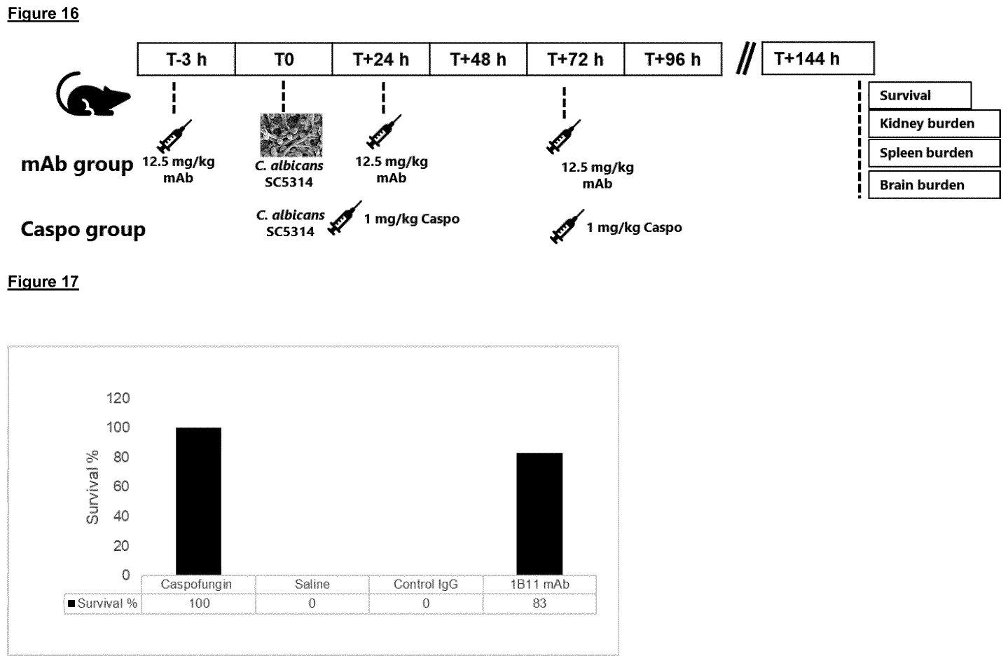

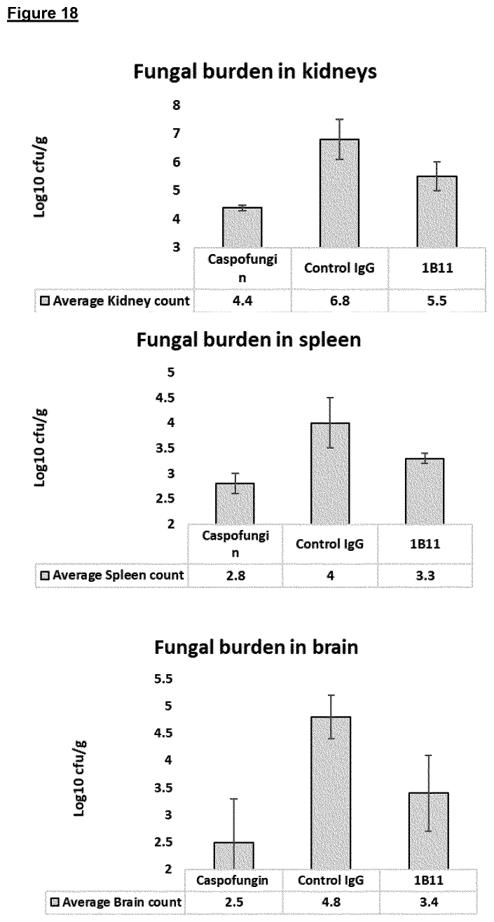

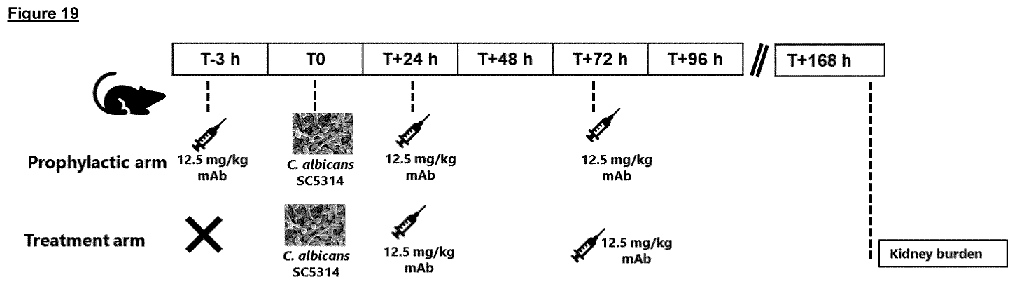

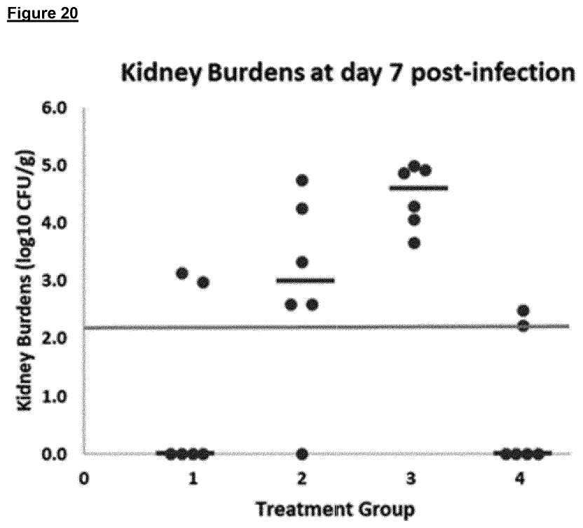

Variations in the cell wall proteome of caspofungin susceptible and resistant strains of C. albicans and C. tropicalis were analysed by employing liquid chromatography-tandem mass spectrometry (LC-MS/MS) of trypsin digested proteins. A schematic diagram representing the various steps involved in sample preparation, peptide generation and cell wall protein identification is shown in FIG. 1.

The following isolates were included:
C. albicans caspofungin susceptible strains-SC5314, CBS8758, ATCC2091, ATCC76615, B17_009053, B17_008835.
C. albicans caspofungin resistant strains-K063-3, B15_004476, B12_007355_1
C. tropicalis caspofungin sensitive isolate-Ct1
C. tropicalis caspofungin resistant isolate-Ct2

These strains were grown in the presence or absence of caspofungin and the cell wall characteristics were studied and compared to detect any changes in protein expression between drug resistant and susceptible isolates.

The protocol for cell wall extractions was modified from (Kapteyn et al., 2000). In some preferred embodiments of the invention, this modified method is used. Briefly, a single colony of test strains were inoculated into YPD broth and incubated overnight at 30° C. with shaking at 200 rpm. Overnight culture was transferred to fresh YPD, grown until exponential phase (OD₆₀₀=0.4˜0.6) in the presence or absence of sub MIC concentration of caspofungin for 90 minutes. Cells were then harvested by centrifugation at 3000×g for 5 minutes and washed once in 10 mM Tris-HCl (pH 7.5).

The mechanical breakage of the cells was accomplished using zirconia/silica 0.5 mm beads in a FastPrep machine (MP Biomedicals). The cell debris containing cell wall was washed 5 times in 1 M NaCl to remove cytoplasmic contamination, resuspended in buffer (500 mM Tris-HCl buffer [pH 7.5], 2% [w/v] SDS, 0.3 M β-mercaptoethanol, and 1 mM EDTA), boiled 3 times at 100° C. for 10 minutes and freeze-dried. The pellets were digested with trypsin according to the PRIME-XS protocol (“PRIME-XS Protocol NPC In Solution Digestion,” 2013). Mass spectrometry analysis was performed using a Q-Exactive Plus (Thermo Fisher Scientific) and tryptic peptides were identified using the MASCOT searching engine (Matrix Science, n.d.).

The analysis was carried out with Proteome Discoverer 2.2 software (Thermo Fisher Scientific), with the proteins matched from Candida Genome Database (www.candidagenome.org) and a cut-off of at least 2 peptides detected per protein. The Area Under the Curve (AUC) gave a semi-quantitative measure of protein abundances.

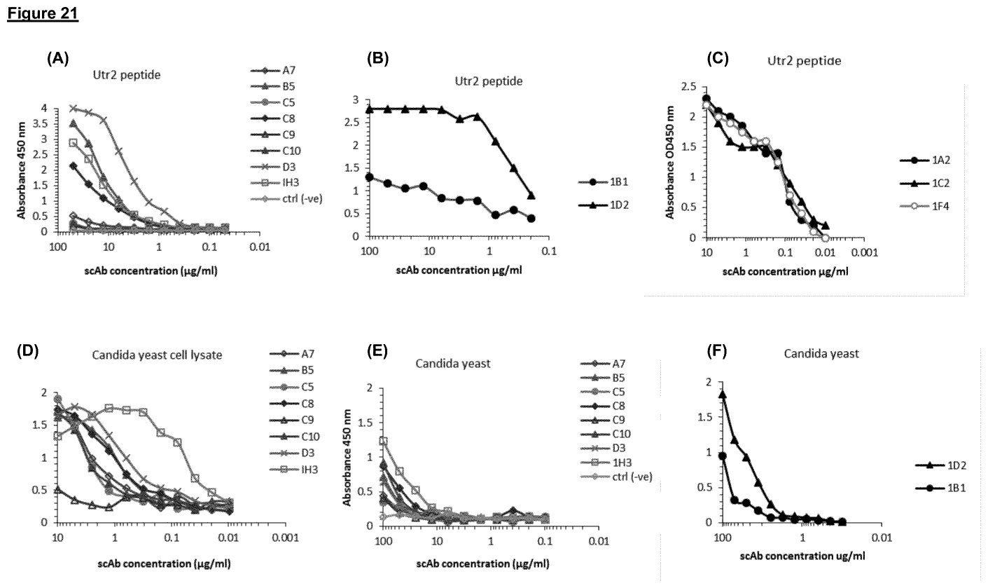

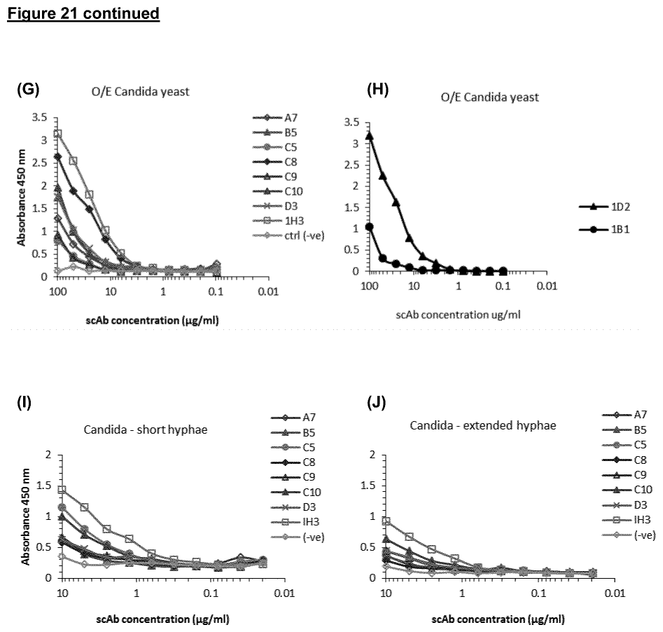

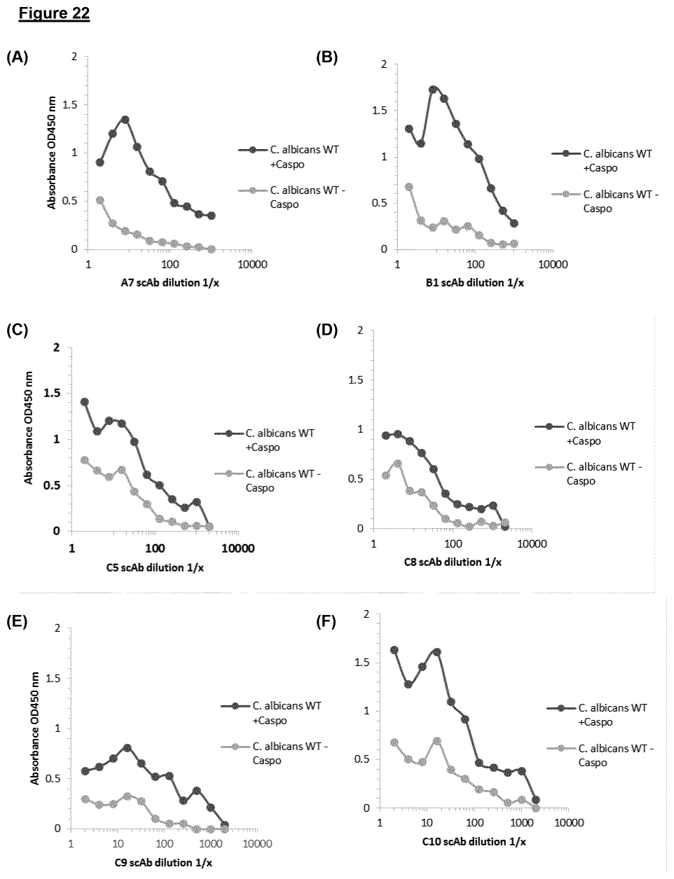

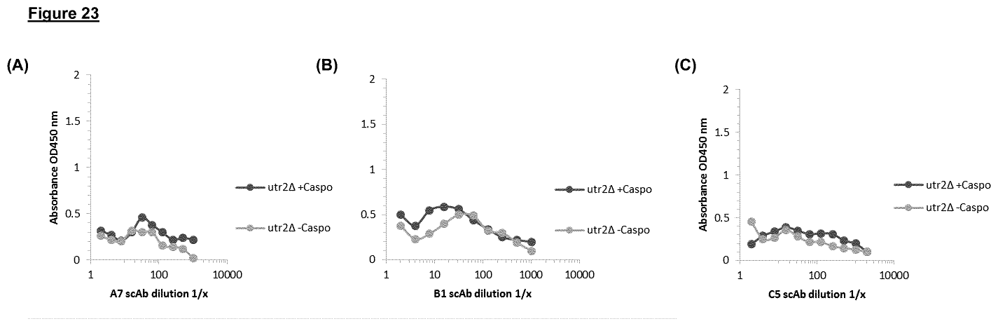

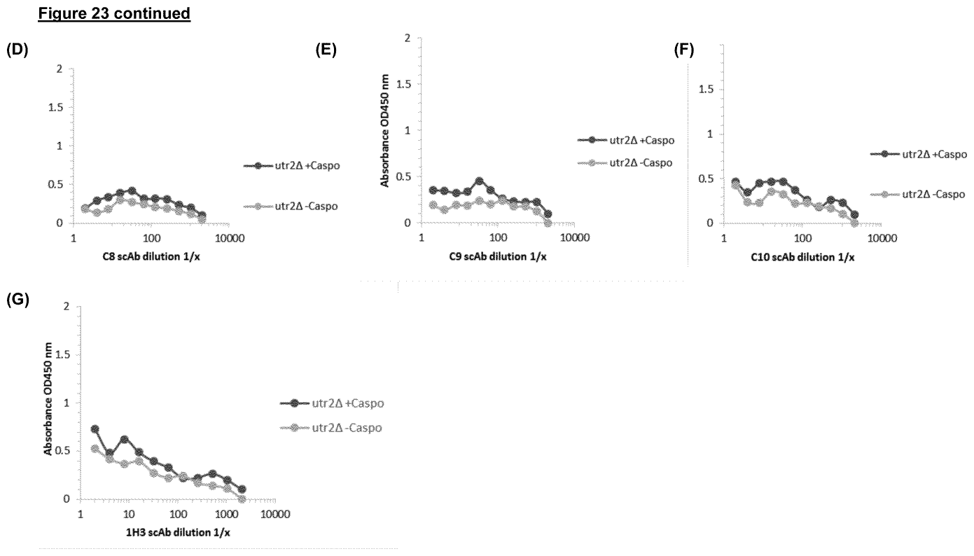

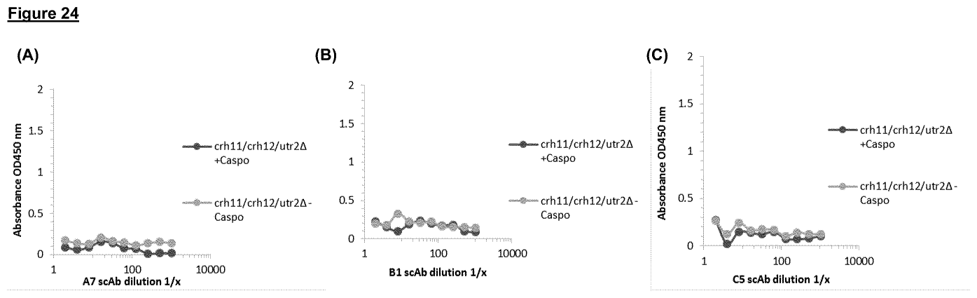

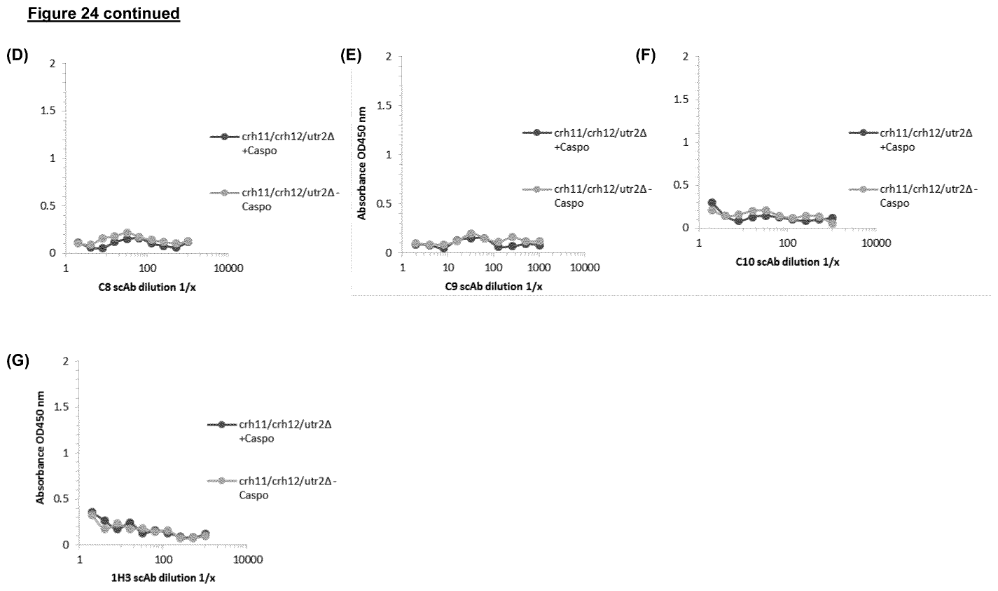

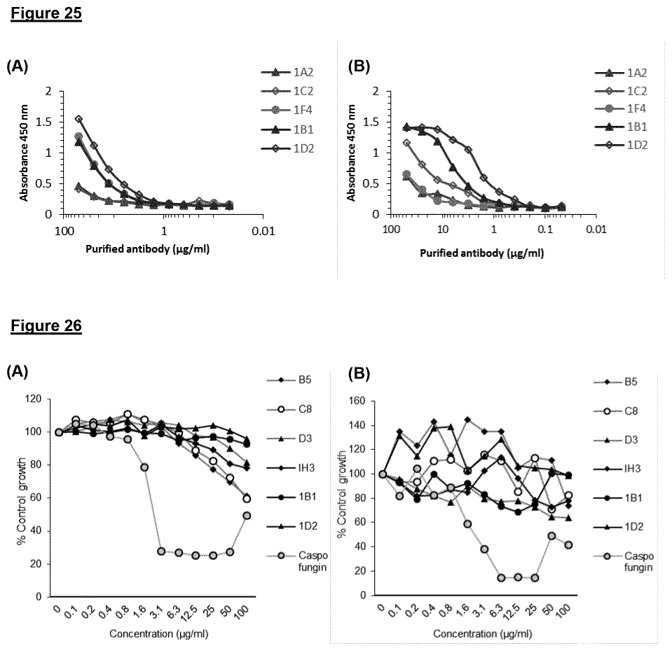

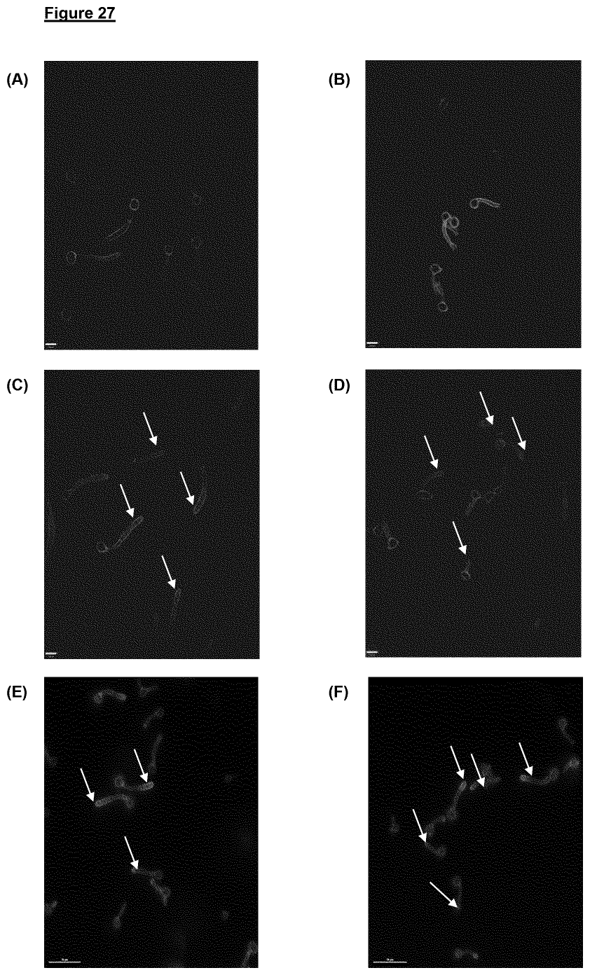

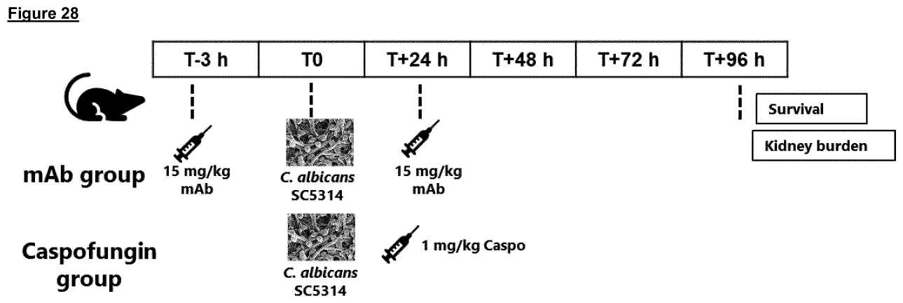

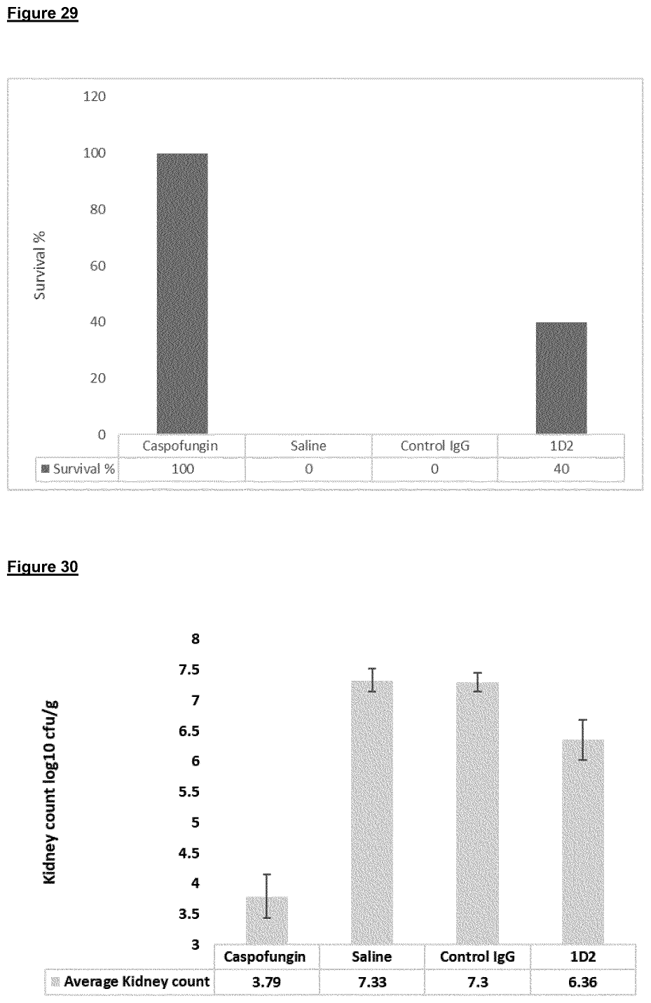

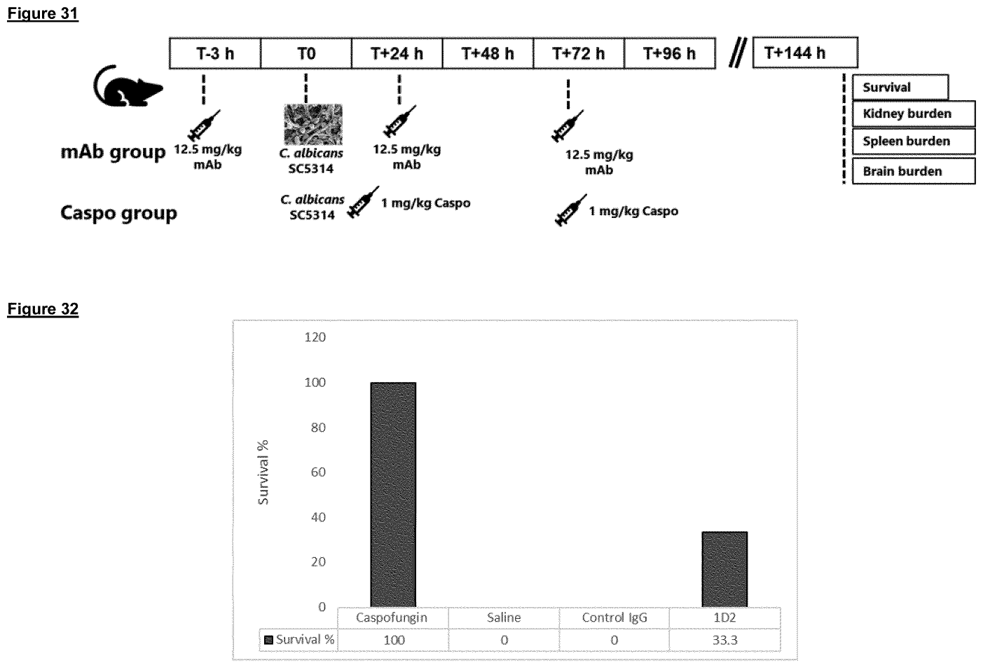

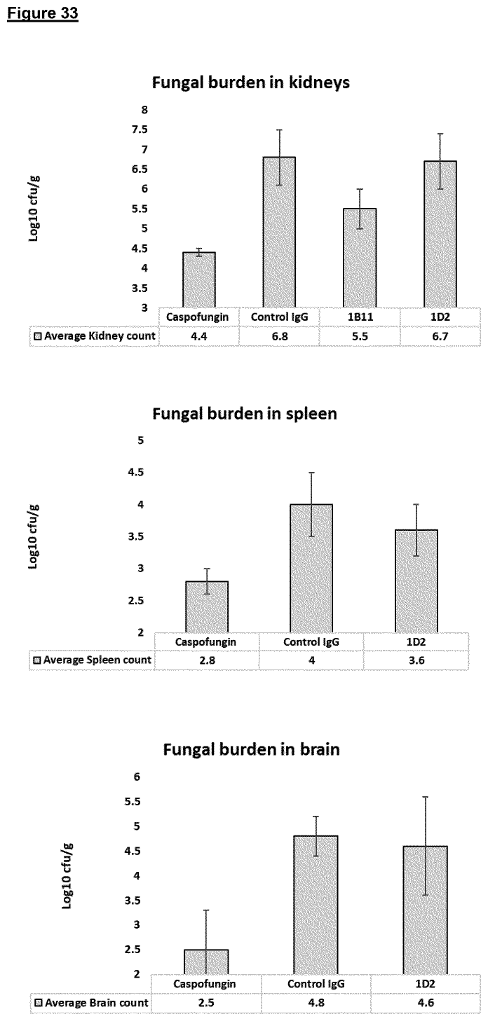

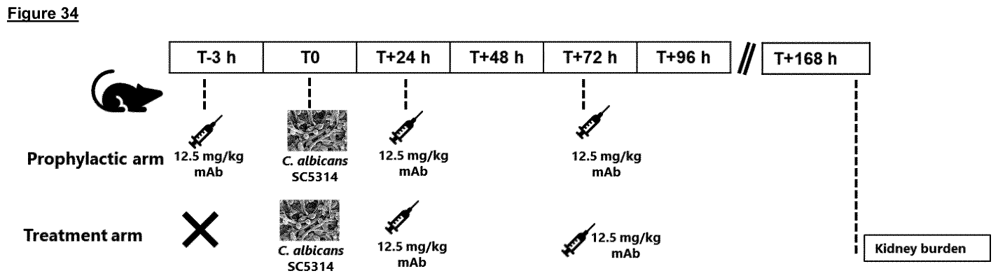

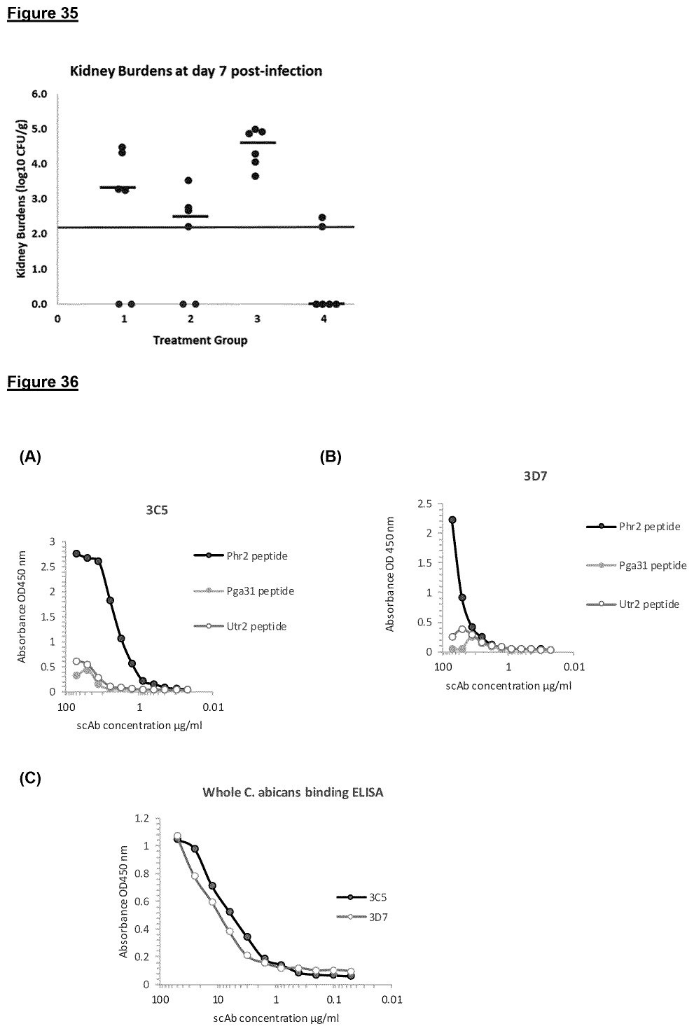

Differences in the expression levels of cell wall proteins especially Pga31, Utr2 and Phr2 and the comparison between resistant and susceptible strains of C. albicans are shown in FIG. 2. Changes in the cell wall proteome of C. tropicalis Ct1 (drug sensitive) and Ct2 (drug resistant) isolates induced with caspofungin is shown in FIG. 3 and the three CWP of interest for antibody generation is highlighted in the table in FIG. 3c.

### Example 2—Methods of Generating Peptide Antigens for the Development of Antifungal Antibodies

Disclosed herein are methods for generating human monoclonal antibodies against fungal cell wall proteins by designing peptide antigens that are surface exposed and accessible for antibody binding. Guided by the proteomics data, the amino acid sequences of peptides that were accessible for trypsin digestion in the cell wall prep were selected and matched with their respective C. albicans cell wall proteins as shown in tables 1-4 (peptides detected by LC-MS/MS shown in bold). From this group, peptide antigens for antibody generation were identified based on their hydropathy and predicted secondary structures.

There is relatively a higher propensity of antibody binding to the regions where beta turn conformations are present in the peptide. Peptide sequences, 30 aa in length, and meeting the above described characteristics in structure and charge were chosen as antigens to represent each of the 3 CWPs for antibody library panning (peptide antigenic regions are shown in boxes in the protein sequences below).

These were custom synthesised and C terminally biotinylated via an additional Lysine residue introduced for Pga31 and Phr2 peptides.

Pga31 peptide sequence-
(SEQ ID NO: 77)
QPLNVGNTVLQLGGSGDGTKVDIAEDGTLS
Utr2 peptide sequence-
(SEQ ID NO: 78)
WPGGDSSNAKGTIEWAGGLINWDSEDIK
Phr2 peptide sequence-
(SEQ ID NO: 79)
QDAGIYVIADLSQPDESINRDDPSWDLDLFER

TABLE 1
Amino acid sequences of the tryptic digested peptides and their associated
CWP Pga31 as identified from the cell wall proteome analysis of
C. albicans SC5314 using LC-MS/MS method. The peptide antigen selected
for antibody generation is shown in box within the protein sequence.

| Peptides | Associated protein sequence as determined by: |
|---|---|
| detected by LC- | www.candidagenome.org/cgi-bin/protein/proteinPage.pl?dbid=CAL0004244 |
| MS/MS | Pga31 (XP_717105.1)-SEQ ID NO: 81 |
| HEGAALNYLFLA | MKFHMRLQKKIFVLEYYIKPDISSFSGKYLFLLFFLFQSHINQLFDYIYFIQKYLIC |
| APGVAENLK | YIMKFLTAASLLTLSSSALAAIKDIQLYAQSSNNEVNDFGISSRHEGAALNYLFL |
| (aa 102-122 of | 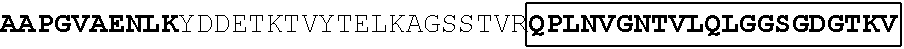 |
| SEQ ID NO: 81) | 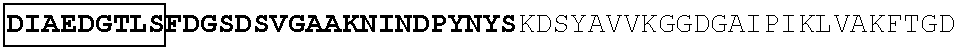 |
| QPLNVGNTVLQ | DKESASSSSSSAAPEPTASSSEAPKETPVYSNSTVTLYTTYCPLSTTITLTVCSD |
| LGGSGDGTK | VCTPTVIETSGSVTVSSVQVPSKTASSEAAPPKTTVDSVSKPAPSGKKPTAAVT |
| (aa 143-162 of | SFEGAANALTGGSVAIAVAAAIGLVF* |
| SEQ ID NO: 81) | Signal peptide: aa 1-41 |
| VDIAEDGTLSFD | TM domain: aa 29-51 |
| GSDSVGAAK | Low complexity: aa 63-78, 220-241, 277-296, and 340-348 (from SMART) |
| (aa 163-183 of | Peptides detected by LC-MS/MS shown in bold |
| SEQ ID NO: 81) | Peptide sequence selected for antibody generation shown in box |
| NINDPYNYSK | |
| (aa 184-193 of | |
| SEQ ID NO: 81) | |

TABLE 2
The amino acid sequences of the tryptic digested peptides and their associated
CWP Utr2 as identified from the cell wall proteome analysis of C. albicans SC5314
using LC-MS/MS method. The peptide antigen selected for antibody
generation is shown in box within the protein sequence.

| Peptides | Associated protein sequence as determined by: |
|---|---|
| detected by LC- | www.candidagenome.org/cgi-bin/protein/proteinPage.pl?dbid=CAL0000104 |
| MS/MS | Utr2 (XP_721748.1)-SEQ ID NO: 82 |
| MSTFQESFDSK | MRFSTLHFAFLATLSSIFTVVAASDTTTCSSSKHCPEDKPCCSQFGICGTGAYCL |
| (aa 75-85 of SEQ | GGCDIRYSYNLTACMPMPRMSTFQESFDSKDKVKEIELQSDYLGNSTEADWVY |
| ID NO: 82) | TGWVDYYDNSLLIQMPNHTTGTVVSSTKYLWYGKVGATLKTSHDGGVVTAFILF |
| IQFSLWPGGDS | SDVQDEIDYEFVGYNLTNPQSNYYSQGILNYNNSRNSSVNNTFEYYHNYEMDW |
| SNAK | 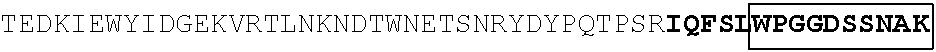 |
| (aa 254-268 of | 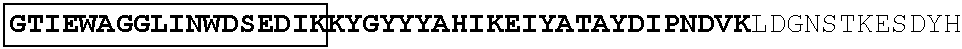 |
| SEQ ID NO: 82) | AFLYNSTDGDASNIMLTTKKTWLGSDDATGFDPQNDDEDSSSNKAQETTITSVS |
| YGYYYAHIK | GSSTITSVKTDSTKKTANVPAQNTAAAAQATAKSSTGTNTYDPSAGVGGFVQD |
| (aa 288-296 of | SKSTDSGSSGSSSQGVANSLNESVISGIFASICLGILSFFM* |
| SEQ ID NO: 82) | Signal peptide: aa 1-23 |
| EIYATAYDIPND | Chitin binding: aa 28-62 |
| VK | Peptides detected by LC-MS/MS shown in bold |
| (aa 297-310 of | Peptide sequence selected for antibody generation shown in box |
| SEQ ID NO: 82) | |
| GTIEWAGGLIN | |
| WDSEDIKK | |
| (aa 269-287 of | |
| SEQ ID NO: 82) | |

TABLE 3
The amino acid sequences of the tryptic digested peptides and their
associated CWP Phr2 as identified from the cell wall proteome analysis of
C. albicans SC5314 using LC-MS/MS method. The peptide antigen selected
for antibody generation is shown in box within the protein sequence.

| Peptides | Associated protein sequence as determined by: |
|---|---|
| detected | www.candidagenome.org/cgi-bin/protein/proteinPage.pl?dbid=CAL0005209 |
| by LC-MS/MS | Phr2 (XP_719043.1)-SEQ ID NO: 83 |
| DIPYLEAVDTNVIR | MLLKSLFPSILAATSFVSSVAAEDLPAIEIVGNKFFYSNNGSQFYIKGIAYQQNN |
| (aa 75-88 of SEQ | 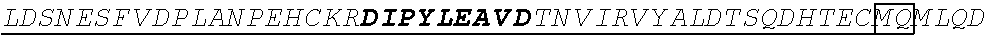 |
| ID NO: 83) | 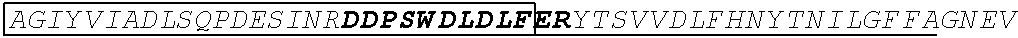 |
| DDPSWDLDLFER | TNKKSNTDASAFVKAAIRDTKAYIKSKGYRSIPVGYSANDDSAIRVSLADYFAC |
| (aa 126-137 of | GDEDEAADFFGINMYEWCGDSSYKASGYESATNDYKNLGIPIFFSEYGCNEV |
| SEQ ID NO: 83) | RPRKFTEVATLFGDQMTPVWSGGIVYMYFEEENNYGLVSIKDNTVSTLKDYS |
| VDSDDYSDLFSYI | YYSSEIKDIHPSSAKASAESASSISRTTCPTNTNNWEASTNLPPTPDKEVCEC |
| CAK | MSASLKCVVDDKVDSDDYSDLFSYICAKIDCDGINANGTTGEYGAYSPCHSK |
| (aa 384-399 of | DKLSFVMNLYYEQNKESKSACDFGGSASLQSAKTASSCSAYLSSAGSSGLG |
| SEQ ID NO: 83) | TVSGTVRTDTSQSTSDSGSGSSSSSSSSSSSSSSGSSGSKSAASIVSVNLLT |
| | KIATIGISIVVGFGLITM* |
| | Signal peptide: aa 1-18 |
| | Glycosyltransferase 72: aa 19-328 |
| | SMART domain SM00768 putative carbohydrate binding: aa 376-464 |
| | Peptides detected by LC-MS/MS shown in bold |
| | Peptide sequence selected for antibody generation shown in box |

TABLE 4
The amino acid sequences of the tryptic digested peptides and their
associated CWP Cht2 as identified from the cell wall proteome analysis of
C. albicans SC5314 using LC-MS/MS method. The peptide antigen selected for
antibody generation is shown in box within the protein sequence.

| Peptides | Associated protein as determined by: |
|---|---|
| detected | www.candidagenome.org/cgi-bin/protein/proteinPage.pl?dbid=CAL0002204 |
| by LC-MS/MS | Cht2 (XP_721807)-SEQ ID NO: 84 |
| TVLLSLGGGVGD | MLSFKSLLAAAVVASSALASASNQVALYWGQNGAGGQERLAQYCQE |
| YGFSDVASATK | 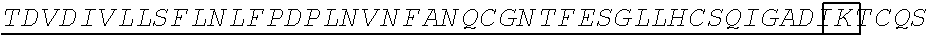 |
| (aa 99-121 of SEQ | 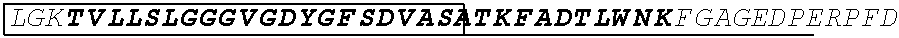 |
| ID NO: 84) | DAVVDGFDFDIEHGGATGYPELATALRGKFAKDTSKNYFLSAAPQCPY |
| FADTLWNK | PDASLGDLLSKVPLDFAFIQFYNNYCSINGQFNYDTWSKFADSAPNKN |
| (aa 122-129 of | IKLFVGVPATSNIAGYVDTSKLSSAIEEIKCDSHFAGVSLWDASGAWL |
| SEQ ID NO: 84) | NTDEKGENFVVQVKNVLNQNACVAPSSSATTQSTTTTSSAVTQSTTT |
| LFVGVPATSNIAG | TSAAITQSATTTSAAVTTKSNQIVTSSSSSSSSIFYGNSTTESSTGIATGf |
| YVDTSK | TVLPTGSNENAATTGSGSNTKLAISTVTDVQKTVITITSCSEHKCVATP |
| (aa 240-258 of | VTTGVVVVTDIDTVYTTYCPLTNSQVYVPVQTVVCTEETCVPSPTSTA |
| SEQ ID NO: 84) | QKPKASTTIKGVEKGQTTSYPVVGTTEGVKKIVTTSAQTVGSSTKYVTI |
| LSSAIEEIK | ELTSTITPVTYPTSVASNGTNTTVPVFTFEGGAAVANSLNSVWFPVPFL |
| (aa 259-267 of | LAAFAF* |
| SEQ ID NO: 84) | Signal peptide: aa 1-19 |
| | Conserved glycoside hydrolase superfamily SSF55445: aa 19-297 |
| | Peptides detected by LC-MS/MS shown in bold |
| | Peptide sequence selected for antibody generation shown in box |

### Example 3—Isolation of Pga31 Specific Binders from a Naïve Human Antibody Library

Phage display technology is a powerful tool for isolating high affinity binders from recombinant antibodies libraries. A naïve human antibody library was biopanned against the surface exposed epitope of C. albicans cell wall protein Pga31. Briefly, in the first round, streptavidin magnetic beads were coated with 500 nM of biotinylated Pga31 peptide and phage particles displaying antibody fragments on their surface allowed to bind to the target.

Bound phage particles were eluted and amplified by infecting E. coli cells. For the second and third rounds of panning, the coating concentration of biotinylated peptide antigen was reduced and rescued phage from previous rounds of panning were allowed to bind to the antigen as outlined in Table 5.

TABLE 5
Showing the biopanning strategy for the isolation of Pga31
specific phage binders using the human antibody library.

| | | | |
|---|---|---|---|
| Pga31 PEPTIDE | Pan 1 - 500 nM | Pan 2 - 100 nM | Pan 2 - 10 nM |
| SELECTION | biotinylated | biotinylated | biotinylated |
| STRATEGY | peptide | peptide | peptide |

Screening of phage monoclonals using ELISA identified two phage binders, which showed specific binding to Pga31 peptide antigen. DNA sequencing revealed diversity in the selected positive phage clones, and these were reformatted into single chain antibodies (scAbs) by cloning their respective scFv gene (VH-linker-VL) into the bacterial expression vector pIMS147 (Hayhurst & Harris 1999) using NcoI and NotI restriction enzymes and standard cloning procedure. Nucleotide and amino acid sequences of these clones is disclosed herein. The linker used in this experiment has the amino acid sequence “LEGGGGGGGGSGGGAS”.

### Example 4—Expression of Reformatted scAbs in Bacterial System and Purification Using Affinity Chromatography

Bacterial stocks of positive clones were grown in Terrific Broth (TB) medium supplemented with PO₄ salts, 100 μg/ml ampicillin and 1% w/v glucose to reach desired cell density, induced with 1 mM IPTG. ScAbs expressed in the periplasm was released using the osmotic shock solution (100 ml 200 mM Tris-HCl-20% sucrose, 200 μl 0.5 M EDTA and 0.5 mg lysozyme followed by 5 mM MgSO₄) and incubating on ice for 15 minutes each.

Recombinant anti-Pga31 scAbs present in crude periplasmic extracts were purified using IMAC columns via binding of hexa Histidine tagged protein to activated Ni-sepharose beads and elution using 200 mM Imidazole. Eluted protein samples were dialysed against 1×PBS pH 7.4 and purity analysed on 4-12% Bis-Tris gels using SDS-PAGE.

All expressed scAbs were found to be 90% pure. Protein concentrations were determined by running a standard scAb of known concentration alongside unknown samples using SDS-PAGE and comparing the intensities of the protein bands using ImageJ. Alternatively, absorbance values at 280 nm were measured using Ultraspec 6300 pro UV/Visible spectrophotometer (Amersham, Biosciences) and final scAb concentrations determined from the values obtained.

### Example 5—Characterisation of the Binding Activity of Anti-Pga31 scAbs

A series of binding ELISAs was performed using biotinylated Pga31 peptide antigen and total cell lysates of WT and pga314 strains of C. albicans. For ELISA using biotinylated Pga31 peptide, plates were pre-coated with 5 μg/ml Streptavidin by incubating at 37° C. for 1 h, washed three times with PBS containing 0.1% tween 20 (PBST) and three times with 1×PBS. The plates were blocked with 2% MPBS, washed as before and 1 μg/ml biotinylated peptide added.

Following incubation at RT for 1 h and washing, scAb samples were added at desired starting concentrations and double diluted across the plate and incubated at room temperature for 1 h. Binding was detected using anti-Human C Kappa HRP conjugated secondary antibody and the resulting immunoreaction was developed by adding SureBlue TMB substrate solution.

The reaction was stopped using 1 M H₂SO₄ and the absorbance values measured using a microplate reader at absorbance 450 nm. For total cell lysate ELISA, C. albicans WT and pga31 single mutant strain overnight cultures were inoculated into fresh YPD at OD₆₀₀ nm of 0.1-0.2, grown for 3-4 h at 30° C. until OD of 0.5-0.6 was reached and then treated with caspofungin (0.032 μg/ml) for 90 min. After 90 min growth cells were harvested, centrifuged for 5 min at 4000 rpm and washed and cell lysate was prepared. This was used to coat ELISA plates at 37° C. for 1 h, plates were washed and blocked with 2% MPBS as before, followed by the two-fold serial dilution of scAb samples. Binding was detected using anti-Human C Kappa HRP conjugated secondary antibody and the resulting immunoreaction was measured as described previously.

Peptide antigen specific binding activity of reformatted Pga31 scAbs-1B11 and 1G4 are shown in FIG. 4. Binding using total cell lysates of C. albicans wild type strain SC3514 treated or untreated with 0.032 μg/ml caspofungin is shown in FIG. 5. An increase in scAb binding was seen in samples from the cells treated with caspofungin. The lack of scAb binding when the mutant strain (pga314) was used is shown in FIG. 6.

Without wishing to be bound by any theory, the inventors believe that Pga31 is overexpressed as a possible remodelling mechanism for maintaining cell wall integrity when the cells are grown in the presence of caspofungin.

### Example 6—Effect of 1B11 scAb on Candida Biofilm Formation

The ability of 1B11 scAb in preventing the formation of biofilm in C. albicans (SC3514) and C. auris (1716) was tested. The growth medium used were RPMI and 20% Foetal Calf Serum (FCS) and the assay was carried out in 96 well microtitre plates. Test antibody 1B11 scAb at a starting concentration of 160 μg/ml was double diluted down the plate and the top row served as positive control with cells only. The final number of cells added to each well was kept constant at 1×10⁵/ml and the plate was incubated at 37° C. for 24 h. Following incubation, the biofilm plate was gently washed two times with PBS to remove non-adhering cells and allowed to dry at room temperature overnight. The biofilm was stained with 100 μl 0.05% crystal violet for 20 min followed by washing with Millipore Q water and 200 ul of 100% ethanol. Contents of each well were transferred to a new plate and absorbance was read at 570 nm wavelength.

At higher concentrations, the scAb was able to inhibit biofilm formation after 24 h incubation (FIG. 7A, B). C. auris does not readily form biofilms, although the cells attach to each other and to the plastic surface of the well. The scAb did not significantly inhibit C. auris growth under biofilm-inducing conditions except at the highest concentration tested (FIG. 7A). C. albicans growth was significantly inhibited especially when cells were grown in 20% foetal calf serum (FIG. 7B).

### Example 7—Antibody Mediated Protection of Galleria mellonella in Candida Infection Models

Galleria mellonella is a reliable infection model to test and compare antibody efficacy by monitoring the survival of the larvae. Ten Galleria mellonella larvae (Livefoods Direct Ltd, Sheffield, UK) with bodyweights of =250-300 mg were used for each test group and standardised inoculum of 5×10⁵ C. albicans and 5×10⁷ C. auris yeast cells/larvae were injected using Hamilton syringe (Cole-Parmer, UK, washed twice with 100% ethanol and sterile dH2O between every 5 injections) via the last pro-leg. Two control groups, one with no injection and the second with PBS injection were also set up. After injection, larvae were incubated for 48-60 h at 37° C. Survival was monitored first after 24 h and then every 6 h. A Kaplan-Meier plot was generated to assess the survival of larvae infected with C. albicans and C. auris, monitoring the survival rate for 60 h (FIG. 8).

The survival percentage is greater in treated larvae compared to the non-treated larvae, without antibody. Survival data showed a significant difference in the killing of larvae by C. auris. Larvae infected with C. auris and then treated with anti-Pga31 antibody survived significantly more than the non-treated larvae (FIG. 8A). Larvae infected with C. albicans had similar survival rates when treated with the scAb; caspofungin only had marginal effect (FIG. 8B).

In addition, the fungal burden of various test groups was calculated by measuring the number of colony forming units (CFU) as an indicator of fungal growth within the larvae and early fungicidal/fungistatic activity of the antibody. After 24 h incubation, there was a notable inhibition of growth for C. auris (1716) when compared to the non-treatment group and these results were statistically significant including the early time point group at 4 h. (FIG. 9A). For C. albicans (SC5314) a lower level of growth inhibition was achieved compared to the non-treatment group and these results were statistically significant in 24 h group (FIG. 9B).

### Example 8—Reformatting 1B11 scAb to Human-Mouse Chimeric Monoclonal Antibody (mAb)

ScAb clone 1B11 was reformatted into human-mouse (IgG2a) chimeric mAb by inserting the antibody VH and VL genes into the dual plasmid eukaryotic vector system (pEE2a) encoding constant heavy and light chain genes of mouse IgG2a. The resulting recombinant mAb was expressed in a transient mammalian expression system. Based on the DNA sequencing data, VH and VL genes of 1B11 was custom synthesised by introducing the cloning sites BssHII and BstEII (for VH gene) and BssHII and XhoI (for VL gene) at their 5 and 3′ end respectively (GeneArt custom gene synthesis service by Thermofisher). Custom synthesised VH and VL genes were cloned into respective eukaryotic expression vectors pEE2aMH (encoding mouse IgG2a constant regions) and pEE2aML (mouse K constant domain) using standard restriction enzyme digestion and ligation steps. Purified DNA fragments were used to transform electrocompetent E. coli TG1 cells for plasmid propagation. DNA sequencing of extracted plasmid confirmed successful reformatting into sheep-mouse chimeric mAb. Large scale preparation of heavy and light chain plasmids was performed (Qiagen Plasmid Mega kit) and used to transfect Human Embryonic Kidney (HEK293F) cells grown in suspension using polyethylenimine (PEI). The transfected cells were grown for 8 days before harvesting the cell culture supernatant which was then purified using Protein A beads following standard protocols.

Purified mAbs were confirmed for binding using biotinylated Pga31 peptide antigen and performing an ELISA as described previously. The starting concentration of 1B11 mAb was 50 μg/ml and a non-related IgG2a mAb was added to the plate as negative control. Binding was detected using anti-mouse IgG Fc region specific HRP conjugated secondary antibody and the resulting immunoreaction was developed as before (FIG. 10A).

The specificity of 1B11 mAb binding was confirmed by performing a cross-reactivity assay and repeating the ELISA using Phr2 and Utr2 peptides (FIG. 10B). In addition, whole cell binding ELISAs using wild type C. albicans (SC5314) yeast and hyphae forms and an over expression strain of Pga31 also confirmed the ability of 1B11 mAb to access and recognise its binding epitope on the surface of C. albicans whole cells (FIG. 10C-E).

### Example 9—Immunofluorescence Staining of C. albicans Using 1B11 mAb

The ability of 1B11 mAb to specifically bind to its target expressed on the cell surface of Candida albicans was tested by immunofluorescence staining. C. albicans WT was attached on a poly-L-lysine glass slide as described previously. To induce hyphal growth the cells were grown in DMEM medium containing 10% heat-inactivated foetal calf serum (FCS) and incubated at 37° C. for 3 h. Cells were washed three times in Dulbecco's phosphate-buffered saline (DPBS) and fixed with 4% paraformaldehyde at room temperature for 15 minutes. After washing, cells were blocked with 1.5% BSA and incubated for 1 h. Cells were washed as before and stained with 1B11 mAb at 10 μg/ml for 1 h at room temperature. After washing, cells were stained with FITC-labelled anti-mouse antibody (1 μg/ml) and incubated at room temperature for 1 h. Cells were washed again and stained with 25 μg/ml of Calcofluor white (CFW) to illuminate chitin-containing structures. Mounting medium was added with a coverslip before images were taken using an UltraVIEW® VoX spinning disk confocal microscope (Perkin Elmer, Waltham, Mass, USA). Calcofluor white stain was viewed at lower brightness (×1) and FITC stain was viewed at higher brightness (×8).

Fluorescent staining was observed with 1B11 mAb binding which was confirmed to be localised on C. albicans cell surface in the merged image in FIG. 11.

### Example 10—Macrophage Interaction Assay

In invasive fungal infections, macrophages play a key role in the initial recognition, ingestion and elimination of C. albicans cells as part of the host's innate defence mechanism. This phagocytic process happens in distinct stages of macrophage migration, recognition of fungal pathogen associated molecular patterns (PAMPs), engulfment of bound cells and finally the processing of engulfed cells by forming phagolysosomes. Studies have reported that the macrophage migration is dependent on C. albicans cell wall glycosylation pattern and the engulfment is more effective with the yeast form than hyphae. More interestingly, the length of hyphae influenced engulfment above the cut off value of 20 μm, where uptake events were slower and defective for longer hyphae (Lewis et al., 2012). It is well established that monoclonal antibodies can act as opsonins and mark the invading Candida cells for destruction by binding to the Fcγ receptors present on the cell surface of phagocytes, thereby enhancing phagocytosis and resulting in efficient killing (Ulrich & Ebel, 2019).

The ability of anti-Pga31 mAb to act as an opsonin for macrophage recruitment and mediating phagocytosis was investigated by setting up a macrophage interaction assay using 1B11 mAb. For phagocytosis experiments, J774.1 mouse macrophages grown in supplemented DMEM medium (200 U/ml penicillin/streptomycin, 2 mM L-glutamine and 10% heat-inactivated FCS) were seeded at a density of 1×10⁵ cells/well in an 8-well glass-based imaging dish and incubated overnight at 37° C., 5% CO2. Immediately before the addition of C. albicans yeast cells, the supplemented DMEM was replaced with pre-warmed supplemented CO2-independent medium to ensure macrophages remained viable during the analysis of C. albicans interactions. Wild-type C. albicans (SC5314) yeast cells at a density of 3×10⁵ cells/well were pre-coated with or without 50 μg/ml Pga31 mAb in pre-warmed supplemented CO2-independent DMEM and incubated at 37° C. with gentle shaking for 40 minutes. This induced yeast cells to filament approximately 20 minutes into the experiment upon initial interactions. A positive control mouse antibody (C. albicans mAb H74E, Invitrogen) was also included in the assay. Video microscopy experiments were performed using an UltraVIEW® VoX spinning disk confocal microscope in a 37° C. chamber. Images were captured at 1 min intervals over a 2 h period. Three different videos were recorded for each antibody or control experiment, and subsequent analysis was conducted using Volocity 6.3 imaging analysis software (PerkinElmer). Measurements taken include C. albicans yeast cell uptake, defined as the number of C. albicans yeast cells taken up by an individual macrophage and the length of intracellular and extracellular hyphae at multiple time points.

For 1B11 mAb treated C. albicans cells, the average length of macrophage engulfed intracellular hyphae was lower compared to the positive control antibody post 60 min (FIG. 12 left).

### Example 11—Testing the Therapeutic Efficacy of 1B11 mAb in a C. albicans Mouse Infection Model

### Study 1

In study 1, the protective effect of mAb 1B11 as a prophylactic agent with single dosing before and after the administration of fungal inoculum was investigated. Female BALB/c mice, 7-9 weeks old were purchased from Envigo Ltd. and randomly assigned to groups of 6 for treatment and control. The Candida albicans inoculum was prepared by growing strain SC5314 in NGY medium for 16 h, shaking at 30° C. Cells were harvested and washed with saline, then counted and resuspended in saline to provide an inoculum of approx. 2×10⁴ CFU/g mouse body weight in 100 μl. Mice were infected intravenously.

Actual inoculum level was determined by plating dilutions of the inoculum on Sabouraud dextrose agar and incubating overnight at 30° C. It was determined to be 2.2×10⁴ CFU/g mouse body weight.

Animals were allocated into four main groups as explained below. The treatment dose of 1B11 mAb was 15 mg/kg per mouse. All treatments were administered intraperitoneally (IP) in 150 μl saline.

The study plan is shown in FIG. 13 and is as follows.
Group 1B11-1B11—Pre-treatment with 1B11 mAb at 3 h before infection, followed by treatment at 24 h post-infection.
Group Saline only—Saline injections at 3 h before infection, followed by treatment at 24 h post-infection
Group caspofungin—caspofungin treatment (1 mg/kg) at 24 h post-infection
Group isotype control antibody—Pre-treatment with Isotype control (mouse IgG2a) at 3 h before infection, followed by treatment at 24 h post-infection

Mice were monitored and weighed every day and were culled on day 4 post-infection and the kidneys removed aseptically. Kidneys were split in half, with one half of each kidney used to determine burdens, one half frozen on OCT and one half fixed in formalin at 4° C. Burdens were determined by plating out kidney homogenate and counting colonies after 48 h growth at 30° C. Statistical comparisons were carried out using IBM SPSS version 24.

Efficacy of antibody therapy treatment was measured by fungal burden and survival. Comparing across all groups (Kaplan-Meier log-rank statistics), there was a highly significant difference between the groups (p<0.0001). Removing the caspofungin treatment group from the analysis, still showed a highly significant difference (p=0.003). Comparing the saline only group, isotype control antibody group, and group 1B11-1B11, there was a highly significant difference between these groups (p=0.002). Survival of mice in treatment and control groups is shown in FIG. 14.

The kidney burdens in treatment and control groups 4 days post infection is shown in FIG. 15. Comparing the percentage weight change (day 0-2, data not shown) and kidney fungal burdens, there were highly significant differences between the groups (p=0.001 for both parameters) (Kruskal-Wallis test). Comparing the saline only group, control antibody group, and 1B11 mAb group, there was a significant difference between these groups for kidney burdens (p=0.015) and weight change (p=0.011).

Comparing 1B11 mAb group to the control IgG group, there was a significant difference in kidney organ burdens on day 4 (p=0.017) (Mann Whitney U comparison). Caspofungin-treated mice showed a significant decrease in kidney organ burdens (p=0.004), compared to saline treated mice. There was no difference in kidney fungal burdens between control IgG-treated mice and saline-treated mice (p=0.247).

### Study 2

In study 2, the protective effect of mAb 1B11 as a prophylactic agent with single dosing 3 hours before infection followed by two dosing 24 h and 72 h post infection was investigated Animals were allocated into four main groups as explained previously. The treatment dose of 1B11 mAb was 12.5 mg/kg per mouse. All treatments were administered intraperitoneally (IP) in 150 μl saline.

The study plan is shown in FIG. 16 and is as follows:
Group 1B11-1B11—Pre-treatment with 1B11 mAb at 3 h before infection, followed by treatment at 24 h and 72 h post-infection.
Group Saline only—Saline injections at 3 h before infection, followed by treatment at 24 h and 72 h post-infection
Group caspofungin—caspofungin treatment (1 mg/kg) at 24 h and 72 h post-infection
Group isotype control antibody—Pre-treatment with Isotype control (mouse IgG2a) at 3 h before infection, followed by treatment at 24 h and 72 h post-infection

Mice were monitored and weighed every day and were culled on day 6 post-infection and the kidneys removed aseptically. Burdens in kidneys, brain and spleen were determined by plating out organ homogenates and counting colonies after 48 h growth at 30° C.

Efficacy of antibody therapy treatment was measured by survival percentage and fungal burden in the kidneys, brain and spleen. Survival of mice in treatment and control groups is shown in FIG. 17 and organ burdens in FIG. 18.

In study 1, 1B11 mAb was administered as a single dose prophylactic followed by single dose therapy 24 h post infection. In contrast, for study 2, two doses of B11 mAb was administered 24 and 72 hours post infection in addition to the single dose prophylactic. Double dose treatment has clearly shown increase in final survival % compared to the single dose (83% vs 66%). The benefit of double dosing was also reflected in kidney fungal burdens with a further three log 10 reduction in the number of fungal cells achieved in mice receiving two doses of mAb post infection compared to single dosing (Study 1 control-7.3 log 10 cfu/g, 1B11-5.73 log 10 cfu/g VS Study 3 control-4.8 log 10 cfu/g, 1B11-1.9 log 10 cfu/g).

For Utr2 targeting antibodies (1D2 and 1H3 mAbs) (see Example 17), double dose treatment using 1D2 did not result in any further increase in final survival % compared to the single dose (single dose-40% vs double dose-33%). Interestingly, based on fungal burden in the kidneys, two doses of 1H3 mAb in treatment only group indicated a superior effect compared to study groups 1 and 2 where the mAb was administered prophylactically-3 h followed by single or double dosing post infection (Study 1 control-7.3 log 10 cfu/g, 1D2 mAb-6.36 log 10 cfu/g VS Study 2 control-6.8 log 10 cfu/g, 1D2 mAb-6.7 log 10 cfu/g VS Study 3 control-4.8 log 10 cfu/g, 1H3 mAb-2.2 log 10 cfu/g).

Based on the final survival and fungal burden in organs, an increased therapeutic efficacy is observed in groups of mice receiving two doses of experimental mAbs compared to single dosing. Due to the extensive cell wall remodelling events happening during an in vivo infection, CWPs like Pga31 and Utr2 are overexpressed which can favour mAb binding and mark fungal cells for clearance via opsonisation and phagocytosis by macrophages and neutrophils. With the reported half-life of murine IgG2a isotype in mice falling in the range of 3-5 days, the administration of a second mAb dose 72 h post infection would have resulted in an increased antibody concentration in associated organs during the study course. Additional experiments may establish the circulating serum levels of antifungal mAbs administered intraperitoneally and their tissue distribution patterns (PK and PD profiles) compared to systemic antifungal agents such as caspofungin. However the serum half-lives of therapeutic human IgGs are often reported in the region of 21-28 days as opposed to 10-24 h the existing antifungal classes which will translate to a reduced dosing frequency for mAbs in the clinic.

### Study 3

In study 3, the protective effect of mAb 1B11 as a prophylactic agent with single dosing 3 hours before infection followed by two dosing 24 h and 72 h post infection was investigated.

In addition, a treatment only arm with the administration of 1B11 mAb 24 h and 72 h post infection was also included.

Animals were allocated into four main groups as explained below. The treatment dose of 1B11 mAb was 12.5 mg/kg per mouse. All treatments were administered intraperitoneally (IP) in 200 μl saline.

The study plan is shown in FIG. 19 and is as follows:
Group 1—Pre-treatment with 1B11 mAb at 3 h before infection, followed by treatment at 24 h and 72 h post-infection.
Group 2—Injected with saline at 3 h before infection, treatment with 1B11 at 24 h and 72 h post-infection.
Group 3—Saline injections at all three time points
Group 4—caspofungin treatment (1 mg/kg) at 24 h and 72 h post-infection

Mice were monitored and weighed every day and were culled on day 7 post-infection and the kidneys removed aseptically. Kidneys were split in half, with one half of each kidney used to determine burdens, one half frozen on OCT and one half fixed in formalin at 4° C. Burdens were determined by plating out kidney homogenate and counting colonies after 48 h growth at 30° C. Statistical comparisons were carried out using IBM SPSS version 24.

No mice developed severe symptoms during treatments, infection or during the monitoring procedures.

The kidney burdens in various treatment and control groups 7 days post infection is shown in FIG. 20. The detection limit for kidney burden determination was 2.3 log 10 CFU/g; negative counts were assigned a value of one-half log below the detection limit (i.e. 1.8 log₁₀ CFU/g).

Distribution of kidney burdens across the different groups was compared by Kruskal-Wallis non-parametric test, where there was a significant difference found (P=0.003). There was a highly significant difference when the saline treated group was compared to 1B11 pre and post treated and caspofungin groups. There was a significant difference between saline treated and 1B11 post treated groups. In summary, the antifungal activity and protective effect of 1B11 mAb when administered intraperitoneally in a mouse model of C. albicans infection was demonstrated by reduced kidney burdens in treatment groups 7 days post infection.

### Example 12—Isolation of Utr2 Specific Binders from a Naïve Human Antibody Library

A similar strategy was adopted for the generation of monoclonal antibodies to the second cell wall protein target Utr2. Based on the C. albicans cell wall proteomics data, five peptide sequences detected by LC-MS/MS method were identified to be the part of Utr2 protein and a peptide sequence of 28 amino acids—WPGGDSSNAKGTIEWAGGLINWDSEDIK—was selected for biopanning. As described previously, for the first round of panning, streptavidin magnetic beads were coated with 500 nM of biotinylated Utr2 peptide and phage particles displaying antibody fragments on their surface allowed to bind to the target. Bound phage particles were eluted and amplified by infecting E. coli cells. For second and third round of panning, the coating concentration of biotinylated peptide antigen was reduced and rescued phage from previous rounds of panning were allowed to bind to the antigen as outlined in Table 6.

TABLE 6
Showing the biopanning strategy for the isolation of
Utr2 specific phage binders using the human library.

| | | | |
|---|---|---|---|
| Utr2 PEPTIDE | Pan 1 - 500 nM | Pan 2 - 100 nM | Pan 2 - 10 nM |
| SELECTION | biotinylated | biotinylated | biotinylated |
| STRATEGY | peptide | peptide | peptide |

Screening of phage monoclonals using ELISA identified several phage binders, which showed specific binding to Utr2 peptide antigen. DNA sequencing revealed diversity in the selected positive phage clones, and these were reformatted into single chain antibodies (scAbs) by cloning their respective scFv gene (VH-linker-VL) into the bacterial expression vector pIMS147 using NcoI and NotI restriction enzymes and standard cloning procedure. The linker used in this experiment has the amino acid sequence “LEGGGGSGGGGSGGGAS”.

### Example 13—Characterisation of the Binding Activity of Anti-Utr2 scAbs

Positive phage binders from Utr2 peptide panning were reformatted into scAbs and expressed in a bacterial system as described previously. A series of binding ELISA was performed using biotinylated Utr2 peptide antigen and total cell lysates of WT and utr2Δ strains of C. albicans. ELISA methodology is the same as described for anti-Pga31 binders.

Peptide antigen binding activity of reformatted Utr2 scAbs-A7, B5, C5, C8, C9, C10, 1H3, 1B1 and 1D2 are shown in FIG. 21A-C. Binding using whole cell lysates of C. albicans wild type strain SC3514 treated or untreated with 0.032 μg/ml caspofungin is shown in FIG. 22. An increase in scAb binding was seen in samples from the cells treated with caspofungin. The lack of scAb binding towards C. albicans mutant strains utr2Δ and utr2Δ: crh11Δ: crh124 are shown in FIGS. 23 and 24.

### Example 14—Cross Reactivity of Utr2 scAbs to Aspergillus fumigatus Hyphal Cells

C. albicans Utr2 shares certain degree of sequence similarity with S. cerevisiae Utr2, S. cerevisiae Crh1 and an Aspergillus fumigatus allergen Aspf9, with highly conserved putative glycosyl hydrolase domains (Alberti-Segui et al., 2004). The cross-reactivity of selected Utr2 scAbs to A. fumigatus was tested by performing a whole cell binding ELISA using C. albicans SC5314 and A. fumigatus AF293/FGSC1100 strains. To induce hyphal formation, wells of a MaxiSorp flat-bottom 96 well plate was coated with approximately 10⁶ overnight grown cells diluted in DMEM or RPMI-1640 modified medium containing 10% heat-inactivated foetal calf serum (FCS) and incubated at 37° C. for 2-20 h. Cells were washed three times in PBS+0.05% Tween, blocked with 1.5% bovine serum albumin (BSA), and incubated at room temperature for 1 h. Cells were washed as before and a starting concentration of 100 μg/ml scAb in 1% blocking buffer was added in duplicate in doubling dilutions and incubated at room temperature for 1.5 h. After washing, cells were incubated with anti-human C kappa peroxidase antibody as described previously. Following incubation for 45 min at room temperature, wells were washed and stained with tetramethylbenzidine (TMB) and read at an absorbance of 450 nm in a VersaMax microplate reader (Molecular Devices, USA).

ScAbs 1B1, 1C2 and 1D2 were found to be cross-reactive to A. fumigatus hyphae (FIG. 25) suggesting that these antibodies could be useful for targeting Candida and Aspergillus cell wall proteins.

### Example 15—Candida albicans Growth Inhibition Assay

The antifungal activity of Utr2 scAbs was assessed by measuring the growth of fungal cells in microtitre plates. A growth assay was carried out with C. albicans WT strain SC5314 and using caspofungin as a positive control. A starting concentration of 100 μg/ml was prepared for the test scAbs (B5, C8, D3, 1H3, 1B1 and 1D2) and caspofungin and these were double diluted in a 96 well microtitre plate in designated wells. The wells were inoculated using 50 μl 2×10⁶ cells/ml diluted in RPMI-1640 medium and incubated for 24 h and 48 h at 37° C. Cell growth was determined by measuring optical density at 405 nm in a spectrophotometer.

After 24 h, clones C8 and B5 showed 40% growth inhibition at top concentration of 100 μg/ml. No further reduction was achieved following further incubation. Clone D3, on the other hand, was able to inhibit growth approximately 40% after 48 h incubation. None of the other clones could slow down or inhibiting the growth of C. albicans cells using this assay (FIG. 26).

### Example 16—Immunofluorescence Staining of C. albicans Using Utr2 scAbs

Overnight grown C. albicans WT and Utr2 overexpression strains were diluted and left to adhere on a poly-L-lysine glass slide for 1 h. To induce hyphal growth the cells were grown in DMEM medium containing 10% heat-inactivated foetal calf serum (FCS) and incubated at 37° C. for 2 h. Cells were washed three times in Dulbecco's phosphate-buffered saline (DPBS) and fixed with 4% paraformaldehyde at room temperature for 15 minutes. After washing, cells were blocked with 1.5% BSA and incubated for 1 h. Cells were washed as before and stained with Utr2 scAbs at 10 μg/ml for 1 h at room temperature. After washing, cells were stained with FITC-labelled anti-human kappa light chain antibody (1 μg/ml) and incubated at room temperature for 1 h. Cells were washed again and stained with 25 μg/ml of Calcofluor white (CFW) to illuminate chitin-containing structures. Mounting medium was added with a coverslip before images were taken using an UltraVIEW® Vox spinning disk confocal microscope (Perkin Elmer, Waltham, Mass, USA). Calcofluor white stain was viewed at lower brightness (×1) and FITC stain was viewed at higher brightness (×8). Calcofluor white stained the chitin rich cell wall as well as septa of both C. albicans strains.

There was minimal staining of C. albicans WT and overexpressing strains with 1B1 scAb (FIG. 27A-B). However, 1D2 scAb appeared to have specifically stained the hyphal tips and bud surface of C. albicans cells in both strains tested and not the yeast cells or septa (FIG. 27 C-D). The immunofluorescence staining was repeated for 1D2 scAb with higher concentration of scAb (50 μg/ml) and FITC-labelled anti-human kappa light chain antibody (5 μg/ml). This confirmed the staining of 1D2 scAb to the hyphae (white arrow), especially tips of the hyphae and bud surface of C. albicans wild type and Utr2 overexpression strains (FIG. 27 E-F).

### Example 17—Reformatting 1H3 and 1D2 scAb to Human-Mouse Chimeric Monoclonal Antibody (mAb) and Testing its Therapeutic Efficacy in a Mouse Model of Fungal Infection

The Utr2 scAbs 1H3 and 1D2 were reformatted into human-mouse (IgG2a) chimeric mAb by cloning its VH and VL genes into the previously described in-house dual plasmid eukaryotic vector system (pEE2a) encoding constant heavy and light chain genes of mouse IgG2a. The reformatted mAb was expressed in a transient HEK expression system and purified using Protein A column chromatography. A series of binding ELISAs was performed using biotinylated Utr2 peptide antigen and total cell lysates of WT C. albicans to confirm the binding activity of reformatted 1H3 and 1D2 mAbs.

### Study 1

The protective effect of 1D2 and IH3 mAbs were evaluated in a series of mouse models of infection as described previously.

In study 1, the protective effect of mAb 1D2 as a prophylactic agent with single dosing before and after the administration of fungal inoculum was investigated. Female BALB/c mice were infected with Candida albicans WT strain SC5314 as before and the animals were allocated into four treatment groups as explained below. The treatment dose of 1D2 mAb was 15 mg/kg per mouse. All treatments were administered intraperitoneally (IP) in 200 μl in saline.

The study plan is shown in FIG. 28 and is as follows:
Group 1D2-1D2—Pre-treatment with 1D2 mAb at 3 h before infection, followed by treatment at 24 h post-infection.
Group Saline only—Saline injections at 3 h before infection, followed by treatment at 24 h post-infection
Group caspofungin—caspofungin treatment (1 mg/kg) at 24 h post-infection
Group isotype control antibody—Pre-treatment with Isotype control (mouse IgG2a) at 3 h before infection, followed by treatment at 24 h post-infection

Mice were monitored and weighed every day and were culled on day 4 post-infection and the kidneys removed aseptically. Kidneys were split in half, with one half of each kidney used to determine burdens, one half frozen on OCT and one half fixed in formalin at 4° C. Burdens were determined by plating out kidney homogenate and counting colonies after 48 h growth at 30° C. Statistical comparisons were carried out using IBM SPSS version 24.

Efficacy of antibody therapy treatment was measured by fungal burden and survival. Comparing across all groups (Kaplan-Meier log-rank statistics), there was a highly significant difference between the groups (p<0.0001). Removing the caspofungin treatment group from the analysis, still showed a highly significant difference (p=0.003). Comparing the saline only group, control antibody group, and group 1D2-1D2, there was a lack of statistically significant difference between these groups (p=0.096).

Survival of mice in treatment and control groups is shown in FIG. 29.

The kidney burdens in treatment and control groups 4 days post infection is shown in FIG. 30. Comparing the percentage weight change (day 0-2, data not shown) and kidney fungal burdens, there were highly significant differences between the groups (p=0.001 for both parameters) (Kruskal-Wallis test). Comparing the saline only group, control antibody group, and 1D2 mAb group, there was a significant difference between these groups for weight change (p=0.023), but not for kidney burdens (p=0.377).

### Study 2

In study 2, the protective effect of mAb 1D2 as a prophylactic agent with single dosing 3 hours before infection followed by two dosing 24 h and 72 h post infection was investigated Animals were allocated into four main groups as explained previously. The treatment dose of mAbs were 12.5 mg/kg per mouse. All treatments were administered intraperitoneally (IP) in 150 μl saline.

The study plan is shown in FIG. 31 and is as follows:
Group 1D2-1D2—Pre-treatment with 1D2 mAb at 3 h before infection, followed by treatment at 24 h and 72 h post-infection.
Group Saline only—Saline injections at 3 h before infection, followed by treatment at 24 h and 72 h post-infection
Group caspofungin—caspofungin treatment (1 mg/kg) at 24 h and 72 h post-infection
Group isotype control antibody—Pre-treatment with Isotype control (mouse IgG2a) at 3 h before infection, followed by treatment at 24 h and 72 h post-infection

Mice were monitored and weighed every day and were culled on day 6 post-infection and the kidneys removed aseptically. Burdens in kidneys, brain and spleen were determined by plating out organ homogenates and counting colonies after 48 h growth at 30° C.

Efficacy of antibody therapy treatment was measured by survival percentage and fungal burden in the kidneys, brain and spleen. Survival of mice in treatment and control groups is shown in FIG. 32 and organ burdens in FIG. 33.

For Utr2 targeting antibodies (1D2 and 1H3 mAbs), double dose treatment using 1D2 did not result in any further increase in final survival % compared to the single dose (single dose —40% vs double dose—33%). Interestingly, based on fungal burden in the kidneys, two doses of 1H3 mAb in treatment only group indicated a superior effect compared to study groups 1 and 2 where the mAb was administered prophylactically—3 h followed by single or double dosing post infection (Study 1 control—7.3 log 10 cfu/g, 1D2 mAb—6.36 log 10 cfu/g VS Study 2 control—6.8 log 10 cfu/g, 1D2 mAb—6.7 log 10 cfu/g VS Study 3 control—4.8 log 10 cfu/g, 1H3 mAb—2.2 log 10 cfu/g).

### Study 3

In study 3, the protective effect of mAb 1H3 as a prophylactic agent with single dosing 3 hours before infection followed by two dosing 24 h and 72 h post infection was investigated. In addition, a treatment only arm with the administration of 1H3 mAb 24 h and 72 h post infection was also included.

Animals were allocated into four main groups as explained below. The treatment dose for the mAb was 12.5 mg/kg per mouse. All treatments were administered intraperitoneally (IP) in 200 μl saline.

The study plan is shown in FIG. 34 and is as follows:
Group 1—Pre-treatment with 1H3 mAb at 3 h before infection, followed by treatment at 24 h and 72 h post-infection.
Group 2—Injected with saline at 3 h before infection, treatment with 1H3 at 24 h and 72 h post-infection.
Group 3—Saline injections at all three time points
Group 4—caspofungin treatment (1 mg/kg) at 24 h and 72 h post-infection

Mice were monitored and weighed every day and were culled on day 7 post-infection and the kidneys removed aseptically. Kidneys were split in half, with one half of each kidney used to determine burdens, one half frozen on OCT and one half fixed in formalin at 4° C. Burdens were determined by plating out kidney homogenate and counting colonies after 48 h growth at 30° C. Statistical comparisons were carried out using IBM SPSS version 24. No mice developed severe symptoms during treatments, infection or during the monitoring procedures.

The kidney burdens in various treatment and control groups 7 days post infection is shown in FIG. 35. The detection limit for kidney burden determination was 2.3 log 10 CFU/g; negative counts were assigned a value of one-half log below the detection limit (i.e. 1.8 log 10 CFU/g). Distribution of kidney burdens across the different groups was compared by Kruskal-Wallis non-parametric test, where there was a significant difference found (P=0.003). There was a highly significant difference when the saline treated group was compared to 1H3 post treated group and caspofungin group. With saline and 1H3 pre and post treatment groups, the difference was approaching significance.

There was no significant difference between 1H3 pre and post compared to 1H3 post treatment only group. In summary, the antifungal activity and protective effect of 1H3 mAb when administered intraperitoneally in a mouse model of C. albicans infection was demonstrated by reduced kidney burdens in treatment groups 7 days post infection.

### Example 18—Isolation of Phr2 Specific scAbs and their Biochemical Characterisation

Similar to the strategies adopted for the isolation of antibodies to Pga31 and Utr2, phage binders and subsequent scAbs were generated against the third cell wall protein target Phr2.

The peptide sequence-QDAGIYVIADLSQPDESINRDDPSWDLDLFER was selected for biopanning. Several phage monoclonals binding to the peptide antigen were identified and based on their unique amino acid sequences reformatted into scAbs as described previously.

Binding of selected scAbs to the Phr2 peptide and whole C. albicans cells is shown in FIG. 36. Sequences of shortlisted clones are disclosed herein.

### SELECTED REFERENCES

Gow, N. A. R., Latge, J. P. and Munro, C. A. (2017) The fungal cell wall: structure, biosynthesis and function Microbiol Spectr May; 5 (3). doi: 10.1128/microbiolspec.FUNK-0035-2016.
Plaine A, Walker L, Da Costa G, Mora-Montes H M, Mckinnon A, Gow N A, Gaillardin C, Munro C A, Richard M L. (2008) Functional Analysis of Candida Albicans GPI-anchored Proteins: Roles in Cell Wall Integrity and Caspofungin Sensitivity. Fungal Genet Biol October; 45 (10): 1404-14. doi: 10.1016/j.fgb.2008.08.003.
Kapteyn J C, Hoyer L L, Hecht J E, Müller W H, Andel A, Verkleij A J, Makarow M, Van Den Ende H, Klis F M (2000) The Cell Wall Architecture of Candida Albicans Wild-Type Cells and Cell Wall-Defective Mutants Mol Microbiol February; 35 (3): 601-11. doi: 10.1046/j. 1365-2958.2000.01729.x.
Pitarch, A., Nombela, C., Gil, C. (2008) Cell Wall Fractionation for Yeast and Fungal Proteomics Methods in Molecular Biology 425:217-39 doi: 10.1007/978-1-60327-210-0_19
Castillo, L., Calvo, e., Aartínez, A., Ruiz-Herrera, J., Valentin, E., Lopez, J., and Sentandreu, R. (2008) A study of the Candida albicans cell wall proteome. Proteomics 8, 3871-3881 doi: 10.1002/pmic.200800110
Ene, I., Adya, A., Wehmeier, S., Brand, A., MacCallum, D., Gow, N., Brown, A. (2012) Host Carbon Sources Modulate Cell Wall Architecture, Drug Resistance and Virulence in a Fungal Pathogen Cell Microbiol September; 14 (9): 1319-35. doi: 10.1111/j. 1462-5822.2012.01813.x.
Pardini, G., De Groot, P., Coste, A., Karababa, M., Klis, F., de Koster, C., Sanglard, D. (2006) The CRH Family Coding for Cell Wall Glycosylphosphatidylinositol Proteins With a Predicted Transglycosidase Domain Affects Cell Wall Organization and Virulence of Candida Albicans J Biol Chem December 29; 281 (52): 40399-411. doi: 10.1074/jbc.M606361200. Epub 2006 Oct. 30.
Alberti-Segui, C., Morales, A., Xing, H., Kessler, M., Willins, D., Weinstock, K., Cottarel, G., Fechtel, K., Rogers, B (2004) Identification of Potential Cell-Surface Proteins in Candida Albicans and Investigation of the Role of a Putative Cell-Surface Glycosidase in Adhesion and Virulence Yeast March; 21 (4): 285-302. doi: 10.1002/yea. 1061.
Farrer, R., Voelz, K., Henk, D., Johnston, S., Fisher, M., May, R. and Cuomo C. (2016) Microevolutionary traits and comparative population genomics of the emerging pathogenic fungus Cryptococcus gattii Philos Trans R Soc Lond B Biol Sci. December 5; 371 (1709): 20160021. doi: 10.1098/rstb.2016.0021
Mühlschlegel, F. A. and Fonzi, W. A. (1997) PHR2 of Candida Albicans Encodes a Functional Homolog of the pH-regulated Gene PHR1 With an Inverted Pattern of pH-dependent Expression Mol Cell Biol October; 17 (10): 5960-7. doi: 10.1128/mcb.17.10.5960.
Hayhurst, A. and Harris, W. (1999) Escherichia Coli SKP Chaperone Coexpression Improves Solubility and Phage Display of Single-Chain Antibody Fragments Protein Expr Purif April; 15 (3): 336-43. doi: 10.1006/prep. 1999.1035.
Lewis, L., Bain, J., Lowes, C., Gillespie, C., Rudkin, F., Gow, N., Lars-Erwig, L. (2012) Stage Specific Assessment of Candida albicans Phagocytosis by Macrophages Identifies Cell Wall Composition and Morphogenesis as Key Determinants PLOS Pathogens Volume 8 Issue 3 e1002578 doi.org/10.1371/journal.ppat. 1002578
Ulrich, S. and Ebel, F. (2020) Monoclonal Antibodies as Tools to Combat Fungal Infections. J. Fungi 6, 22.

Sequence Annex

Key for antibody amino acid sequences:

| | |
|---|---|
| First bold underlined region in each sequence: | VH-CDR1 |
| Second bold underlined region in each sequence: | VH-CDR2 |
| Third bold underlined region in each sequence: | VH-CDR3 |
| Fourth bold underlined region in each sequence: | VL-CDR1 |
| Fifth bold underlined region in each sequence: | VL-CDR2 |
| Sixth bold underlined region in each sequence: | VL-CDR3 |

Pga31 antibody sequences
1B11 NUCLEOTIDE SEQUENCE (SEQ ID NO: 1)
GAGGTGCAGCTGTTGGAGTCTGGGGGAGGCGTGGTCCAGCCTGGGAGGTCCCTGAGA
CTCTCCTGTGCAGCCTCTGGATTCACCTTCAGTAGCTATGGCATGCACTGGGTCCGCC
AGGCTCCAGGCAAGGGGCTGGAGTGGGTGGCAGTTATATCATATGATGGAAGTAATAA
ATACTATGCAGACTCCGTGAAGGGCCGATTCACCATCTCCAGAGACAATTCCAAGAACA
CACTGTATCTTCAAATGAACAGCCTGAGAGCCGAGGACACGGCCGTGTATTACTGTGC
GAGAGATCGTAGTGGGTGGGGATCCCTTGACTACTGGGGCCAGGGCACCCTGGTCAC
CGTCTCCTCACTCGAGGGTGGAGGCGGTTCAGGCGGAGGTGGCTCTGGCGGTGGCG
CTAGCGATATTGTGATGACTCAGTCTCCTGACTCCCTGGCTGTGTCTCTGGGCGAGAG
GGCCACCATCAACTGCAAGTCCAGCCAGAATGTTTTATACAGCTCCAACAATAAGAACT
ACTTAGCTTGGTACCAGCAGAAATCAGGACAGCCTCCTAAGCTGCTCATTTACTGGGCA
TCTACCCGGGAATCCGGGGTCCCTGACCGATTCAGTGGCAGCGGGTCTGGGACAGAT
TTCACTCTCACCATCAGCAGCCTGCAGGCTGAAGATGTGGCAGTTTATTACTGTCAGCA
ATATTACAGTACTCCTCGAACTTTTGGCCAGGGGACCAAGGTGGAGATCAAA
1B11 AMINO ACID SEQUENCE (SEQ ID NO: 2)
EVQLLESGGGVVQPGRSLRLSCAASGFTFSSYGMHWVRQAPGKGLEWVAVISYDGSNKY
YADSVKGRFTISRDNSKNTLYLQMNSLRAEDTAVYYCARDRSGWGSLDYWGQGTLVTVS
SLEGGGGSGGGGSGGGASDIVMTQSPDSLAVSLGERATINCKSSQNVLYSSNNKNYLAW
YQQKSGQPPKLLIYWASTRESGVPDRFSGSGSGTDFTLTISSLQAEDVAVYYCQQYYSTPR
TFGQGTKVEIK
1G4 NUCLEOTIDE SEQUENCE (SEQ ID NO: 3)
GAGGTGCAGCTGTTGGAGTCTGGGGGAGGCTTGGTTCAGCCTGGGGGGTCCCTGAGA
GTCTCCTGTGCAGCCTCTGGATTCACCTTTAACAGCTATGCCATGAGCTGGGTCCGCC
AGGCTCCAGGGAAGGGGCTGGAGTGGGTCTCAACTGTTAGTGGAAGTGGTGGTAGCA
CATACTACGCAGACTCCGTGAAGGGCCGGTTCACCATCTCCAGAGACAATTCCAAGAA
CACGCTGTATCTGCAAATGAACAGCCTGAGAGCTGAGGACACGGCTGTGTATTACTGT
GCGAGAGGGTACTTCGATCTCTGGGGCCGTGGAACCCTGGTCACCGTCTCCTCACTCG
AGGGTGGAGGCGGTTCAGGCGGAGGTGGCTCTGGCGGTGGCGCTAGCGACATCCAG
ATGACCCAGTCTCCATCCTCCCTGTCTGCATCTGTAGGAGACAGAGTCACCATCACTTG
CCGGGCAAGTCAGAGCATTAGCAGCTATTTAAATTGGTATCAGCAGAAACCAGGGAAA
GCCCCTAAGCTCCTGATCTATGCTGCATCCAGTTTGCAAAGTGGGGTCCCATCAAGGTT
CAGTGGCAGTGGATCTGGGACAGATTTCACTCTCACCATCAGCAGTCTGCAACCTGAA
GATTTTGCAACTTACTACTGTCAACAGACTTACACCACCCCGCTCACTTTCGGCGGAGG
GACCAAGGTGGAAATCAAA
1G4 AMINO ACID SEQUENCE (SEQ ID NO: 4)
EVQLLESGGGLVQPGGSLRVSCAASGFTFNSYAMSWVRQAPGKGLEWVSTVSGSGGST
YYADSVKGRFTISRDNSKNTLYLQMNSLRAEDTAVYYCARGYFDLWGRGTLVTVSSLEGG
GGSGGGGSGGGASDIQMTQSPSSLSASVGDRVTITCRASQSISSYLNWYQQKPGKAPKLL
IYAASSLQSGVPSRFSGSGSGTDFTLTISSLQPEDFATYYCQQTYTTPLTFGGGTKVEIK
Utr2 antibody sequences
1A2 NUCLEOTIDE SEQUENCE (SEQ ID NO: 5)
CAGGTTCAGCTTGTGCAGTCTGGGGCTGAGGTGAACAAGCCTGGGGCCTCAGTGAAG
GTTTCCTGCAAGGCTTCTGGATACACCTTCACTAGCTATGCTATGCATTGGGTGCGCCA
GGCCCCCGGACAAAGGCTTGAGTGGATGGGATGGATCAACGCTGGCAATGGTAACAC
AAAATATTCACAGAAGTTCCAGGGCAGAGTCACCATTACCAGGGACACATCCGCGAGC
ACAGCCTACATGGAGCTGAGCAGCCTGAGATCTGAAGACACGGCTGTGTATTACTGTG
CGAGGGGCGGAGCAGCAGCTGGTTACTACATGGACGTCTGGGGCAAAGGAACCCTGG
TCACCGTCTCCTCACTCGAGGGTGGAGGCGGTTCAGGCGGAGGTGGCTCTGGCGGTG
GCGCTAGCAATTTTATGCTGACTCAGCCCCACTCTGTGTCGGAGTCTCCGGGGAAGAC
GGTAACCATCTCCTGCACCCGCAGCAGTGGCAGCATTGCCAGCAACTATGTGCAGTGG
TACCAGCAGCGCCCGGGCAGTTCCCCCACCACTGTGATCTATGACGATAACCAAAGAC
CCTCTGGGGTCCCTGATCGGTTCTCTGCCTCCATCGACAGCTCCTCCAACTCTGCCTC
CCTCACCATCTCTGGACTGAAGACTGAGGACGAGGCTGACTACTACTGTCAGTCTTATG
ATAGCAGCATCGTGGTATTCGGCGGAGGGACCAAGCTGACCGTCCTAGGT
1A2 AMINO ACID SEQUENCE (SEQ ID NO: 6)
QVQLVQSGAEVNKPGASVKVSCKASGYTFTSYAMHWVRQAPGQRLEWMGWINAGNGNT
KYSQKFQGRVTITRDTSASTAYMELSSLRSEDTAVYYCARGGAAAGYYMDVWGKGTLVT
VSSLEGGGGSGGGGSGGGASNFMLTQPHSVSESPGKTVTISCTRSSGSIASNYVQWYQQ
RPGSSPTTVIYDDNQRPSGVPDRFSASIDSSSNSASLTISGLKTEDEADYYCQSYDSSIVVF
GGGTKLTVLG
A7 NUCLEOTIDE SEQUENCE (SEQ ID NO: 7)
CAGGTGCAGCTGGTGGAGTCTGGGGGAGGCGTGGTCCAGCCTGGGAGGTCCCTGAG
ACTCTCCTGTGCAGCCTCTGGATTCACCTTCAGTAGCTATGGCATGCACTGGGTCCGG
CAAGCTCCAGGGAAGGGCCTGGAGTGGGTCTCAGGTATTAGTTGGAATAGTGGTAGCA
TAGGCTATGCGGACTCCGTGAAGGGCCGATTCACCATCTCCAGAGACAATTCCAAGAA
CACGCTGTATCTGCAAATGAACAGCCTGAGAGCTGAGGACACGGCTGTGTATTACTGT
GCGACTAAGTACGGTATGGACGTCTGGGGCCAAGGGACAATGGTCACCGTCTCTTCAC
TCGAGGGTGGAGGCGGTTCAGGCGGAGGTGGCTCTGGCGGTGGCGCTAGCCAGTCT
GTGCTGACTCAGCCACCCTCGGTGTCCAAGACCTTGAGACAGACCGCCACACTCACCT
GCACTGGGAGCAGCAGCAATGTTGCCAACCAAGGAGCAACTTGGCTGCAGCAGCACC
AGGGCCACCCTCCCAAACTCCTATCTTACAGGAATAACAACCGGCCCTCAGGGATCTC
AGAGAGATTCTCTGCATCCAGGTCAGGAAGCACTGCCTCCCTGACCATTACTGGACTC
CAGCCTGACGACGAGGCTGACTATTATTGCTCAGCATGGGACAGCAGCCTCAGTGCTT
GGGTGTTCGGCGGAGGGACCAAGCTGACCGTCCTAGGT
A7 AMINO ACID SEQUENCE (SEQ ID NO: 8)
QVQLVESGGGVVQPGRSLRLSCAASGFTFSSYGMHWVRQAPGKGLEWVSGISWNSGSIG
YADSVKGRFTISRDNSKNTLYLQMNSLRAEDTAVYYCATKYGMDVWGQGTMVTVSSLEG
GGGSGGGGSGGGASQSVLTQPPSVSKTLRQTATLTCTGSSSNVANQGATWLQQHQGHP
PKLLSYRNNNRPSGISERFSASRSGSTASLTITGLQPDDEADYYCSAWDSSLSAWVFGGG
TKLTVLG
1B1 NUCLEOTIDE SEQUENCE (SEQ ID NO: 9)
CAGGGTCAGCTGGTGCAGTCTGGGGGAGGCGTGGTCCAGCCTGGGAGGTCCCTGAG
ACTCTCCTGTGCAGCCTCTGGATTCACCTTCAGTAGCTATGCTATGCACTGGGTCCGCC
AGGCTCCAGGCAAGGGGCTGGAGTGGGTGGCAGTTATATCATATGATGGAAGCAATAA
ATACTACGCAGACTCCGTGAAGGGCCGATTCACCATCTCCAGAGACAATTCCAAGAACA
CGCTGTATCTGCAAATGAACAGCCTGAGAGCTGAGGACACGGCTGTGTATTACTGTGC
GAGAATAGCCCATCCCAGGACCAGGGGGGGAAAGGAAGTTGAATACTTCCAGCACTG
GGGCCAGGGAACCCTGGTCACCGTCTCCTCACTCGAGGGTGGAGGCGGTTCAGGCGG
AGGTGGCTCTGGCGGTGGCGCTAGCGATATTGTGATGACCCAGACTCCACTCTCCTCA
CCTGTCACCCTTGGACAGCCGGCCTCCATCTCCTGCAGGTCTAGTCAAAGCCTCGTAT
ACAGTGATGGAAACACCTACTTGAATTGGTTTCAGCAGAGGCCAGGCCAATCTCCAAG
GCGCCTAATTTATAAGGTTTCTAACCGGGACTCTGGGGTCCCAGACAGATTCAGCGGC
AGTGGGTCAGGCACTGATTTCACACTGAAAATCAGCAGGGTGGAGGCTGAGGATGTTG
GGGTTTATTACTGCATGCAAGGTACACACTGGCCTCCCTCGTTCGGCCAAGGGACCAA
GCTGGAGATCAAACGT
1B1 AMINO ACID SEQUENCE (SEQ ID NO: 10)
QGQLVQSGGGVVQPGRSLRLSCAASGFTFSSYAMHWVRQAPGKGLEWVAVISYDGSNK
YYADSVKGRFTISRDNSKNTLYLQMNSLRAEDTAVYYCARIAHPRTRGGKEVEYFQHWGQ
GTLVTVSSLEGGGGSGGGGSGGGASDIVMTQTPLSSPVTLGQPASISCRSSQSLVYSDGN
TYLNWFQQRPGQSPRRLIYKVSNRDSGVPDRFSGSGSGTDFTLKISRVEAEDVGVYYCMQ
GTHWPPSFGQGTKLEIKR
B5 NUCLEOTIDE SEQUENCE (SEQ ID NO: 11)
CAGGTGCAGCTGGTGGAGTCTGGGGGAGGCGTGGTCCAGCCTGGGAGGTCCCTGAG
ACTCTCCTGTGCAGCCTCTGGATTCACCTTCAGTAGCTATGGCATGCACTGGGTCCGC
CAGGCTCCAGGCAAGGGGCTGGAGTGGGTGGCAGTTATATCATATGATGGAAGTAATA
AATACTATGCAGACTCCGTGAAGGGCCGATTCACCATCTCCAGAGACAATTCCAAGAAC
ACGCTGTATCTGCAAATGAACAGCCTGAAAACCGAAGACACGGCCCAGTATTACTGTGT
TAGAGTTCGGAGGTCGGGTATGGCTCGGGGACTTATTGACTACTGGGGCCAGGGCAC
CCTGGTCACCGTCTCCTCACTCGAGGGTGGAGGCGGTTCAGGCGGAGGTGGCTCTGG
CGGTGGCGCTAGCTCCTATGAGCTGACTCAGGACCCTGCTGTGTCTGTGGCCTTGGGA
CAGACAGTCAGGATCACATGCCAAGGAGACATCCTCAGAAGCTATTATGCAAGTTGGTA
CCAGCAGAAGCCAGGACAGGCCCCTGTACTTGTCATCTATGGTAAAAACAACCGGCCC
TCAGGGATCCCAGACCGATTCTCTGGCTCCAGCTCAGGAAACACAGCTTCCTTGACCA
TCACTGGGGCTCAGGCGGAAGATGAGGCTGACTATTACTGTAACTCCCGGGACAGCAG
TGGTAACCATAAGGTGTTCGGCGGAGGGACCAAGGTCACCGTCCTAGGT
B5 AMINO ACID SEQUENCE (SEQ ID NO: 12)
QVQLVESGGGVVQPGRSLRLSCAASGFTFSSYGMHWVRQAPGKGLEWVAVISYDGSNKY
YADSVKGRFTISRDNSKNTLYLQMNSLKTEDTAQYYCVRVRRSGMARGLIDYWGQGTLVT
VSSLEGGGGSGGGGSGGGASSYELTQDPAVSVALGQTVRITCQGDILRSYYASWYQQKP
GQAPVLVIYGKNNRPSGIPDRFSGSSSGNTASLTITGAQAEDEADYYCNSRDSSGNHKVFG
GGTKVTVLG
1C2 NUCLEOTIDE SEQUENCE (SEQ ID NO: 13)
CAGGTCCAGCTTGTGCAGTCTGGGGCTGAGGTGAAGAAGCCTGGGGCCTCAGTGAAG
GTTTCCTGCAAGGCTTCTGGATACACCTTCACTAGCTATGCTATGCATTGGGTGCGCCA
GGCCCCCGGACAAAGGCTTGAGTGGATGGGATGGATCAACGCTGGCAATGGTAACAC
AAAATATTCACAGAAGTTCCAGGGCAGAGTCACCATTACCAGGGACACATCCGCGAGC
ACAGCCTACATGGAGCTGAGCAGCCTGAGATCTGAAGACACGGCTGTGTATTACTGTG
CGAGGGGCGGAGCAGCAGCNGGTTACTACATGGACGTCTGGGGCAAAGGAACCCTGG
TCACCGTCTCCTCACTCGAGGGTGGAGGCGGTTCAGGCGGAGGTGGCTCTGGCGGTG
GCGCTAGCAATTTTATGCTGACTCAGCCCCACTCTGTGTCGGAGTCTCCGGGGAAGAC
GGTAACCATCTCCTGCACCCGCAGCAGTGGCAGCATTGCCAGCAACTATGTGCAGTGG
TACCAGCAGCGCCCGGGCAGTTCCCCCACCACTGTGATCTATGACGATAACCAAAGAC
CCTCTGGGGTCCCTGATCGGTTCTCTGCCTCCATCGACAGCTCCTCCAACTCTGCCTC
CCTCACCATCTCTGGACTGAAGACTGAGGACGAGGCTGACTACTACTGTCAGTCTTATG
ATAGCAGCATCGTGGTATTCGGCGGAGGGACCAAGCTGACCGTCCTAGGT
1C2 AMINO ACID SEQUENCE (SEQ ID NO: 14)
QVQLVQSGAEVKKPGASVKVSCKASGYTFTSYAMHWVRQAPGQRLEWMGWINAGNGNT
KYSQKFQGRVTITRDTSASTAYMELSSLRSEDTAVYYCARGGAAAGYYMDVWGKGTLVT
VSSLEGGGGSGGGGSGGGASNFMLTQPHSVSESPGKTVTISCTRSSGSIASNYVQWYQQ
RPGSSPTTVIYDDNQRPSGVPDRFSASIDSSSNSASLTISGLKTEDEADYYCQSYDSSIVVF
GGGTKLTVLG
C5 NUCLEOTIDE SEQUENCE (SEQ ID NO: 15)
GAGGTGCAGCTGGTGGAGTCTGGGGGAGGCGTGGTCCAGCCTGGGAGGTCCCTGAG
GCTCTCCTGTGCAGCCTCTGGATTCACCTTCAGTAGCTATGCTATGCACTGGGTCCGC
CAGGCTCCAGGCAAGGGGCTGGAGTGGGTGGCAGTTATATCATATGATGGAAGCAATA
AATACTACGCAGACTCCGTGAAGGGCCGATTCACCACCTCCAGAGACAATTCCAAGAA
CACGCTGTATCTGCAAATGAACAGCCTGAGAGCTGAGGACACGGCTGTGTATTACTGT
GCCCGGATAGCAGTGGCTGGTCGATCCCAAAATGTTGACTACTGGGGCCAGGGCACC
CTGGTCACCGTCTCCTCACTCGAGGGTGGAGGCGGTTCAGGCGGAGGTGGCTCTGGC
GGTGGCGCTAGCGATATTGTGATGACACAGACTCCACTCCCCTCACCTGTCACCCTTG
GACAGCCGGCCTCCATCTCCTGCAGGTCTAGTCAAAGCCTCCTACACAGTGATGGAAG
CACCTACTTGAATTGGTTTCACCAGAGGCCAGGCCAATCTCCAAGGCGCCTAATTTATA
AGGTTTCTAGGCGGGACTCTGGGGTCCCAGACAGATTCAGCGGCAGTGGGTCAGGCA
CTGATTTCACACTGAACATCAGCAGGGTGGAGGCTGAGGATGTTGGGGTTTATTACTG
CATGCAAGGTACACGCTGGCCCCCAATCACCTTCGGACAAGGGACACGACTGGAGATT
AAACGT
C5 AMINO ACID SEQUENCE (SEQ ID NO: 16)
EVQLVESGGGVVQPGRSLRLSCAASGFTFSSYAMHWVRQAPGKGLEWVAVISYDGSNKY
YADSVKGRFTTSRDNSKNTLYLQMNSLRAEDTAVYYCARIAVAGRSQNVDYWGQGTLVTV
SSLEGGGGSGGGGSGGGASDIVMTQTPLPSPVTLGQPASISCRSSQSLLHSDGSTYLNWF
HQRPGQSPRRLIYKVSRRDSGVPDRFSGSGSGTDFTLNISRVEAEDVGVYYCMQGTRWP
PITFGQGTRLEIKR
C8 NUCLEOTIDE SEQUENCE (SEQ ID NO: 17)
GAGGTGCAGCTGGTGCAGTCTGGGGCTGAGGTGAAGAAGCCTGGGTCCTCGGTGAAG
GTCTCCTGCAAGGCTTCTGGAGGCACCTTCAGCAGCTATGCTATCAGCTGGGTGCGAC
AGGCCCCTGGACAAGGGCTTGAGTGGATGGGAATAATCAACCCTAGTGGTGGTAGCAC
AAGCTACGCACAGAAGTTCCAGGGCAGAGTCACCATGACCAGGGACACGTCCACGAG
CACAGTCTACATGGAGCTGAGCAGCCTGAGATCTGAGGACACGGCCGTGTATTACTGT
GCGAGAGGAACCAAGGGTAAGGACTACTACTACATGGACGTCTGGGGCAAAGGCACC
CTGGTCACCGTCTCCTCACTCGAGGGTGGAGGCGGTTCAGGCGGAGGTGGCTCTGGC
GGTGGCGCTAGCGACATCCAGATGACCCAGTCTCCATCTTCCCTGTCTGCATCTGTAG
GAGACAGAGTCACCATCACTTGCCGGGCAAGTCAGAGCATTAGCACCTATTTAAATTGG
TATCAGCAGAAGCCAGGGACAGCCCCTAACCTCCTGATCTATGGTGCATCCAATTTGCA
AAGTGGGGTCCCATCAAGGTTCAGTGGCAGTGGATCTGGGACAGATTTCACTCTCACC
ATCAGCAGTCTGCAACCTGAAGATTTTGCAACTTACTACTGTCAACAGAGTTACAGTAC
CCCTCTCACTTTCGGCGGAGGGACCAAAGTGGATATCAAACGT
C8 AMINO ACID SEQUENCE (SEQ ID NO: 18)
EVQLVQSGAEVKKPGSSVKVSCKASGGTFSSYAISWVRQAPGQGLEWMGIINPSGGSTSY
AQKFQGRVTMTRDTSTSTVYMELSSLRSEDTAVYYCARGTKGKDYYYMDVWGKGTLVTV
SSLEGGGGSGGGGSGGGASDIQMTQSPSSLSASVGDRVTITCRASQSISTYLNWYQQKP
GTAPNLLIYGASNLQSGVPSRFSGSGSGTDFTLTISSLQPEDFATYYCQQSYSTPLTFGGG
TKVDIKR
C9 NUCLEOTIDE SEQUENCE (SEQ ID NO: 19)
CAGGTGCAGCTGGTGGAGTCTGGGGGAGGCGTGGTCCAGCCTGGGAGGTCCCTGAG
ACTCTCCTGTGCAGCCTCTGGATTCTCCTTCAGTAGTTATGGTGTGCACTGGGTCCGCC
AGGCTCCAGGCAAGGGGCTGGAGTGGGTGGCAGGTATATCATATGATGGAAACAATAA
ATACTACACAGACTCCGTGAAGGGCCGATTCACCATCTCCAGAGACAATTCCAAGAACA
CGCTGTATCTGCAAATGAACAGCCTGAGAGCTGAGGACACGGCTGTGTATTACTGTGC
GAAAGGGAGGCTATGGATTGACTACTGGGGCCAGGGAACCCTGGTCACCGTCTCCTCA
CTCGAGGGTGGAGGCGGTTCAGGCGGAGGTGGCTCTGGCGGTGGCGCTAGCGATATT
GTGATGACGCAGTCTCCAGCCACCCTGTCTCTGTCCCCAGGGGAAAGAGCCACCCTCT
CCTGCAGGGCCAGTCAGAGTATTGCCAGAAACTTAGCCTGGTACCAGCTCAGACCTGG
CCAGGCTCCCAGGCTCCTCATCTATGGTGCATCAACCAGGGCCACTGGTGTCCCAGAC
AGATTCAGCGGCAGTGGGTCTGGGACAGATTTCACTCTCACCATCAGCAGCCTGCAGG
CAGAAGATGTGGCAGTTTATTTCTGTCAGCAATATGAAACTCTTCCGTACACTTTTGGCC
GGGGGACCAAAGTGGATATCAAACGT
C9 AMINO ACID SEQUENCE (SEQ ID NO: 20)
QVQLVESGGGVVQPGRSLRLSCAASGFSFSSYGVHWVRQAPGKGLEWVAGISYDGNNKY
YTDSVKGRFTISRDNSKNTLYLQMNSLRAEDTAVYYCAKGRLWIDYWGQGTLVTVSSLEG
GGGSGGGGSGGGASDIVMTQSPATLSLSPGERATLSCRASQSIARNLAWYQLRPGQAPR
LLIYGASTRATGVPDRFSGSGSGTDFTLTISSLQAEDVAVYFCQQYETLPYTFGRGTKVDIK
R
C10 NUCLEOTIDE SEQUENCE (SEQ ID NO: 21)
CAGGTCCAGCTTGTGCAGTCTGGGGCTGAGGTGAAGAAGCCTGGGGCCTCAGTGAAG
GTTTCCTGCAAGGCTTCTGGATACACCTTCACTAGCTATGCTATGCATTGGGTGCGCCA
GGCCCCCGGACAGAGGCTTGAGTGGATGGGATGGATCAACGCTGGCAATGATAACAC
AAAATATTCACAGAAGTTCCAGGGCAGAGTCACCATTACCAGGGACACATCCGCGGGC
ACAGCCTACATGGAGATGAGCGGCCTGACATCTGAGGACACGGCTGTGTATTACTGTG
CGAGAGGCACCTACTACATAGACGTCTGGGGCAAAGGAACCCTGGTCACCGTCTCCTC
ACTCGAGGGTGGAGGCGGTTCAGGCGGAGGTGGCTCTGGCGGTGGCGCTAGCAATTT
TATGCTGACTCAGCCCCACTCTGTGTCGGAGTCTGCGGGGAAGACGGTAACCATCTCC
TGCACCCGCAGCAGTGGCAGCATTGCCAGCAACTATGTGCAGTGGTACCAGCAGCGC
CCGGGCAGTTCCCCCACCACTGTGATCTATGAGGATAACCAAAGACCCTCTGGGGTCC
CTGATCGGTTCTCTGGCTCCATCGACAGCTCCTCCAACTCTGCCTCCCTCACCATCTCT
GGACTGAAGACTGAGGACGAGGCTGACTACTACTGTCAGTCTTATGATAGCAGCAATC
AGGTGTTCGGCGGAGGGACCAAGCTGACCGTCCTAGGT
C10 AMINO ACID SEQUENCE (SEQ ID NO: 22)
QVQLVQSGAEVKKPGASVKVSCKASGYTFTSYAMHWVRQAPGQRLEWMGWINAGNDNT
KYSQKFQGRVTITRDTSAGTAYMEMSGLTSEDTAVYYCARGTYYIDVWGKGTLVTVSSLE
GGGGSGGGGSGGGASNFMLTQPHSVSESAGKTVTISCTRSSGSIASNYVQWYQQRPGSS
PTTVIYEDNQRPSGVPDRFSGSIDSSSNSASLTISGLKTEDEADYYCQSYDSSNQVFGGGT
KLTVLG
1D2 NUCLEOTIDE SEQUENCE (SEQ ID NO: 23)
CAGGTTCAGCTGGTGCAGTCTGGGGCTGAGGTGAAGAAGCCTGGGGCCTCAGTGAAG
GTCTCCTGCAAGGCTTCTGGATACAGCTTCACGAATTATGACATCTACTGGGTGCGACA
GGCCACTGGACAAGGGCTTGAGTGGGTGGGATTCATAAATCCGAAGACTGGTAAAACA
GGCTATGCACAGAAGTTCCAGGGCAGAGTCACCATGAGCAGGGACACTTCCATAACCA
CAGCCTACATGGAACTGAACAGCCTGACATCTGAAGACACGGCCGTGTATTACTGTGC
GAGCATTTCCGGATACTGGGGCCAGGGCACCCTGGTCACCGTCTCCTCACTCGAGGG
TGGAGGCGGTTCAGGCGGAGGTGGCTCTGGCGGTGGCGCTAGCGATATTGTGATGAC
CCAGTCTCCACTCTCCCTGCCCGTCACCCTTGGACAGCCGGCCTCCATCTCCTGCAGG
TCTAGTCAAAGCCTCGTATACAGTGATGGAAACACCTACTTGAATTGGTTTCAGCAGAG
GCCAGGCCAATCTCCAAGGCGCCTAATTTATAAGGTTTCTAACCGGGACTCTGGGGTC
CCAGACAGATTCAGCGGCAGTGGGTCAGGCACTGATTTCACACTGAAAATCAGCAGGG
TGGAGGCTGAGGATGCTGGGCTTTATTACTGCATGCAAGGTTCACACTGGCCTCCGAC
TTTTGGCCAGGGGACCAAGGTGGAGATCAAACGT
1D2 AMINO ACID SEQUENCE (SEQ ID NO: 24)
QVQLVQSGAEVKKPGASVKVSCKASGYSFTNYDIYWVRQATGQGLEWVGFINPKTGKTG
YAQKFQGRVTMSRDTSITTAYMELNSLTSEDTAVYYCASISGYWGQGTLVTVSSLEGGGG
SGGGGSGGGASDIVMTQSPLSLPVTLGQPASISCRSSQSLVYSDGNTYLNWFQQRPGQS
PRRLIYKVSNRDSGVPDRFSGSGSGTDFTLKISRVEAEDAGLYYCMQGSHWPPTFGQGTK
VEIKR
D3 NUCLEOTIDE SEQUENCE (SEQ ID NO: 25)
CAAATGCAGCTGGTGCAGTCTGGGGCTGAGGTGAAAAAGCCCGGGGAGTCTCTGAAG
ATCTCCTGTAAGGGTTCTGGATACAGCTTTACCAGCTACTGGATCGGCTGGGTGCGCC
AGATGCCCGGGAAAGGCCTGGAGTGGATGGGGATCATCTATCCTGGTGACTCTGATAC
CAGATACAGCCCGTCCTTCCAAGGCCAGGTCACCATCTCAGCCGACAAGTCCATCAGC
ACCGCCTACCTGCAGTGGAGCAGCCTGAAGGCCTCGGACACCGCCATGTATTACTGTG
CGAGACTTAAGACAGGCGACCAGCTGCCCGATATCTGGGGCCAAGGGACAATGGTCA
CCGTCTCTTCACTCGAGGGTGGAGGCGGTTCAGGCGGAGGTGGCTCTGGCGGTGGCG
CTAGCGATATTGTGATGACGCAGACTCCACTCTCTCTGTCCGTCACCCCTGGACAGCC
GGCCTCCATCTCCTGCAAGTCTAGTCAGAGCCTCCTGCATAGTGATGGAAAGACCTATT
TGTATTGGTACCTGCAGAAGCCAGGCCAGCCTCCACAGCTCCTGATCTATGAAGTTTCC
AACCGGTTCTCTGGAGTGCCAGATAGGTTCAGTGGCAGCGGGTCAGGGACAGACTTCA
CACTGAAAATCAGCCGGGTGGAGGCTGAGGATGTTGGGGTTTATTACTGCATGCAAAC
TATACAGCTTCCTGCCACGTTCGGCGGAGGGACCAAGCTGGAGATCAGACGT
D3 AMINO ACID SEQUENCE (SEQ ID NO: 26)
QMQLVQSGAEVKKPGESLKISCKGSGYSFTSYWIGWVRQMPGKGLEWMGIIYPGDSDTRY
SPSFQGQVTISADKSISTAYLQWSSLKASDTAMYYCARLKTGDQLPDIWGQGTMVTVSSLE
GGGGSGGGGSGGGASDIVMTQTPLSLSVTPGQPASISCKSSQSLLHSDGKTYLYWYLQK
PGQPPQLLIYEVSNRFSGVPDRFSGSGSGTDFTLKISRVEAEDVGVYYCMQTIQLPATFGG
GTKLEIRR
1F4 NUCLEOTIDE SEQUENCE (SEQ ID NO: 27)
GAGGTGCAGCTGTTGGAGTCGGGGGGAGGCTTGGTCCAGCCTGGGGGATCCCTGAGA
CTCTCTTGTGCAGCCTCTGGATTCACCTTCAGCAATTATGGCATAAACTGGGTCCGCCA
GGCTCCAGGGAAGGGGCTGGAGTGGGTCTCAGCTATTAGTGGTAGTGGTGGTAGCAC
ATACTACGCAGACTCCGTGAAGGGCCGGTTCACCATCTCCAGAGACAATTCCAAGAAC
ACGCTGTATCTGCAAATGAACAGCCTGAGAGCCGAGGACACGGCCGTATATTACTGTG
CGAGTCCGATTCGGGGAGTTAAGCAGCACTGGGGCCAGGGCACCCTGGTCACCGTCT
CCTCACTCGAGGGTGGAGGCGGTTCAGGCGGAGGTGGCTCTGGCGGTGGCGCTAGC
CAGTCTGTGCTGACTCAGCCGTCTTCCCTCTCTGCATCTCCTGGAGCATCAGCCAGTCT
CACCTGCACCTTGCGCAGTGGCATCAATGTTGGTACCTACAGGATATACTGGTATCAGC
AGAAGCCAGGGGGTCCTCCCCAGTATCTCCTGACGTCCATATCAGGCTCAAATTACCA
GCAGGGCTCCGGAGTCCCCAGGCGCTTCTCTGCATACAAAGATGCTTCGGCCAATGCA
GGGATTTTACTCATCTCTGGGCTCCAGTCTGAGGATGAGGCTGACTATTATTGTATGAT
TTGGCACAGCAGCGCTGTGGTCTTCGGCGGAGGGACCAAGCTGACCGTCCTAGGT
1F4 AMINO ACID SEQUENCE (SEQ ID NO: 28)
EVQLLESGGGLVQPGGSLRLSCAASGFTFSNYGINWVRQAPGKGLEWVSAISGSGGSTYY
ADSVKGRFTISRDNSKNTLYLQMNSLRAEDTAVYYCASPIRGVKQHWGQGTLVTVSSLEG
GGGSGGGGSGGGASQSVLTQPSSLSASPGASASLTCTLRSGINVGTYRIYWYQQKPGGP
PQYLLTSISGSNYQQGSGVPRRFSAYKDASANAGILLISGLQSEDEADYYCMIWHSSAVVF
GGGTKLTVLG
1H3 NUCLEOTIDE SEQUENCE (SEQ ID NO: 29)
CAGGTGCAGCTGGTGGAGTCTGGGGGAGGCGTGGTCCAGCCTGGGAGGTCCCTGAG
ACTCTCCTGTGCAGCCTCTGGATTCACCTTCAGTAGCTATGGCATGCACTGGGTCCGC
CAGGCTCCAGGCAAGGGGCTGGAGTGGGTGGCAGTTATATCATATGATGGAAGTAATA
AATACTATGCAGACTCCGTGAAGGGCCGATTCACCATCTCCAGAGACAATTCCAAGAAC
ACGCTGTATCTGCAAATGAACAGCCTGAGAGCTGAGGACACGGCTGTGTATTACTGTG
CGAAACTTAGTAGGGAAAATGCTTTTGATATCTGGGGCCAAGGGACAATGGTCACCGT
CTCTTCACTCGAGGGTGGAGGCGGTTCAGGCGGAGGTGGCTCTGGCGGTGGCGCTAG
CGACATCCAGATGACCCAGTCTCCATCCTCCCTGTCTGCATCTGTAGGAGACAGAGTC
ACCATCACTTGCCAGGCGAGTCAGGACATTTGGAAATATGTAAATTGGTATCAGCAGAA
ACCAGAGAAGGCCCCTAAGTCCCTGATCTATGCTGCATCCAGTTTGCAAAGTGGGGTC
CCATCAAGGTTCAGCGGCAGTGGATCTGGGACAGAATTCACTCTCACAATCAGCAGCC
TGCAGCCTGAAGATTTTGCAACTTACTACTGTCAACAGAGTTACAGTATGCCTCGGACG
TTCGGCCAAGGGACCAAGCTGGAGATCAAACGT
1H3 AMINO ACID SEQUENCE (SEQ ID NO: 30)
QVQLVESGGGVVQPGRSLRLSCAASGFTFSSYGMHWVRQAPGKGLEWVAVISYDGSNKY
YADSVKGRFTISRDNSKNTLYLQMNSLRAEDTAVYYCAKLSRENAFDIWGQGTMVTVSSL
EGGGGSGGGGSGGGASDIQMTQSPSSLSASVGDRVTITCQASQDIWKYVNWYQQKPEK
APKSLIYAASSLQSGVPSRFSGSGSGTEFTLTISSLQPEDFATYYCQQSYSMPRTFGQGTK
LEIKR
Phr2 antibody sequences
3C5 NUCLEOTIDE SEQUENCE (SEQ ID NO: 31)
GAGGTGCAGCTGTTGGAGTCTGGGGGAGGCGTGGTCCAGCCTGGGAGGTCCCTGAGA
CTCTCCTGTGCAGCGTCTGGATTCACCTTCAGTAGCTATGGCATGCACTGGGTCCGCC
AGGCTCCAGGCAAGGGGCTGGAGTGGGTGGCAGTTATATCATATGCTGGAAGCAATAA
ATACTACGCAGACTCCGTGAAGGGCCGATTCACCATCTCCAGAGACAATTCCAAGAACA
CGCTGTATCTGCAAATGAACAGCCTGAGAGCTGAGGACACGGCTGTGTATTACTGTGC
GAAAGGTCAGAGAATGGACGTCTGGGGCAAAGGGACAATGGTCACCGTCTCTTCACTC
GAGGGTGGAGGCGGTTCAGGCGGAGGTGGCTCTGGCGGTGGCGCTAGCGACATCCA
GATGACCCAGTCTCCATCCTCCCTGTCTGCATCTGTAGGAGACAGAGTCACCATCACTT
GCCGGGCAAGTCAGAGCATTAGCAGCTATTTAAATTGGTATCAGCAGAAACCAGGGAA
AGCCCCTAAGCTCCTGATCTATGCTGCATCCAGTTTGCAAAGTGGGGTCCCATCAAGGT
TCAGTGGCAGTGGATCTGGGACAGATTTCACTCTCACCATCAGCAGTCTGCAACCTGAA
GATTTTGCAACTTACTACTGTCAACAGAGTTACAGTACCCCTATCACTTTCGGCCCTGG
GACCAAAGTGGATATCAAACGT
3C5 AMINO ACID SEQUENCE (SEQ ID NO: 32)
EVQLLESGGGVVQPGRSLRLSCAASGFTFSSYGMHWVRQAPGKGLEWVAVISYAGSNKY
YADSVKGRFTISRDNSKNTLYLQMNSLRAEDTAVYYCAKGQRMDVWGKGTMVTVSSLEG
GGGSGGGGSGGGASDIQMTQSPSSLSASVGDRVTITCRASQSISSYLNWYQQKPGKAPK
LLIYAASSLQSGVPSRFSGSGSGTDFTLTISSLQPEDFATYYCQQSYSTPITFGPGTKVDIKR
2C7 NUCLEOTIDE SEQUENCE (SEQ ID NO: 33)
GAAGTGCAGCTGGTGCAGTCTGGGGCTGAGGTGAAGAAGCCTGGGGCCTCAGTGAAG
GTCTCCTGCAAGGCTTCTGGATACACCTTCACCGGCTACTATATGCACTGGGTGCGAC
AGGCCCCTGGACAAGGGCTTGAGTGGATGGGACGGATCAACCCTAACAGTGGTGACA
CAAACTATGCACAGAAGTTTCAGGGCAGGGTCACCATGACCAGGGACACGTCCATCAG
CACAGCCTACATGGAGCTGAACAGGCTGAGATCTGACGACACGGCCGTGTATTACTGT
GCGAGAGATCGCGACCGAGGTATGGACGTCTGGGGCCAAGGGACAATGGTCACCGTC
TCTTCACTCGAGGGTGGAGGCGGTTCAGGCGGAGGTGGCTCTGGCGGTGGCGCTAGC
GACATCCAGATGACCCAGTCTCCAGACTCACTGACTTTGTCTCTGGGCGAGAGGGCCA
CCGTCAACTGCGTGTCCAGCCAAAATCTTTTATTCAACTCCAACAAAAAGAACTGCTTAG
CTTGGTATCAGCAAAAAGCAGGACAGCCTCCTAGGCTGGTCATTTACTGGGCATCCAC
CCGGAAATCCGGGGTCCCTGACCGATTCAGTGGCAGCGGGTCTGGGACAGATTTCACT
CTCACCATCAGCAGTCTACAACCTGAAGATTTTGCAACTTACTACTGTCAACGGAGTTA
CAGTTCCCCGTTCACTTTTGGCCAGGGGACCAAGGTGGAGATCAAACGT
2C7 AMINO ACID SEQUENCE (SEQ ID NO: 34)
EVQLVQSGAEVKKPGASVKVSCKASGYTFTGYYMHWVRQAPGQGLEWMGRINPNSGDT
NYAQKFQGRVTMTRDTSISTAYMELNRLRSDDTAVYYCARDRDRGMDVWGQGTMVTVSS
LEGGGGSGGGGSGGGASDIQMTQSPDSLTLSLGERATVNCVSSQNLLFNSNKKNCLAWY
QQKAGQPPRLVIYWASTRKSGVPDRFSGSGSGTDFTLTISSLQPEDFATYYCQRSYSSPFT
FGQGTKVEIKR
1F5 NUCLEOTIDE SEQUENCE (SEQ ID NO: 35)
CAGGTGCAGCTGGTGCAGTCTGGGGCTGAGGTGAAGAAGCCTGGGGCCTCAGTGAAG
GTCTCCTGCAAGGCTTCTGGTTACACCTTTACCAGCTATGGTATCAGCTGGGTGCGACA
GGCCCCTGGACAAGGGCTTGAGTGGATGGGATGGATCAGCGCTTACAATGGTAACACA
AACTATGCACAGAAGCTCCAGGGCAGAGTCACCATGACCACAGACACATCCACGAGCA
CAGCCTACATGGAGCTGAGGAGCCTGAGATCTGACGACACGGCCGTGTATTACTGTGC
GAGAGATTCGGGATATTTTGACTGGTTCCCCTACTACTACTACTACGGTATGGACGTCT
GGGGCCAAGGGACAATGGTCACCGTCTCTTCACTCGAGGGTGGAGGCGGTTCAGGCG
GAGGTGGCTCTGGCGGTGGCGCTAGCAATTTTATGCTGACTCAGCCCCACTCTGTGTC
GGAGTCTCCGGGGAAGACGGTAACCATCTCCTGCACCCGCAGCAGTGGCAGCATTGC
CAGCAACTATGTGCAGTGGTACCAGCAGCGCCCGGGCAGTGCCCCCACCACTGTGAT
CTATGAGGATAACCAAAGACCCTCTGGGGTCCCTGATCGGTTCTCTGGCTCCATCGAC
AGCTCCTCCAACTCTGCCTCCCTCACCATCTCTGGACTGAAGACTGAGGACGAGGCTG
ACTACTACTGTCAGTCTTATGATAGCGTCAATCGGGGAATATTCGGCGGAGGGACCCA
GCTCACCGTTTTAGGT
1F5 AMINO ACID SEQUENCE (SEQ ID NO: 36)
QVQLVQSGAEVKKPGASVKVSCKASGYTFTSYGISWVRQAPGQGLEWMGWISAYNGNTN
YAQKLQGRVTMTTDTSTSTAYMELRSLRSDDTAVYYCARDSGYFDWFPYYYYYGMDVWG
QGTMVTVSSLEGGGGSGGGGSGGGASNFMLTQPHSVSESPGKTVTISCTRSSGSIASNY
VQWYQQRPGSAPTTVIYEDNQRPSGVPDRFSGSIDSSSNSASLTISGLKTEDEADYYCQSY
DSVNRGIFGGGTQLTVLG
1G10 NUCLEOTIDE SEQUENCE (SEQ ID NO: 37)
CAGGTGCAGCTGGTGGAGTCTGGTCCTACGCTGGTGAAACCCACACAGACCCTCACG
CTGACCTGCACCTTCTCTGGGTTCTCACTCAGCACTAGTGGAGTGGGTGTGGGCTGGA
TCCGTCAGCCCCCAGGAAAGGCCCTGGAGTGGCTTGCACTCATTTATCGGAATGAAGA
TAAGCGCTACAGCCCATCTCTGGAGCGCAGGCTCACCATCACCAAGGACACCTCCAAA
AACCAGGTGGCCCTTACAATGACCGACATGGCCCCTGAGGACACAGCCACATATTACT
GTGCACACAGGGCGGCTACAGCAGCTGTCCTAGACTACTGGGGCCAGGGAACCCTGG
TCACCGTCTCCTCACTCGAGGGTGGAGGCGGTTCAGGCGGAGGTGGCTCTGGCGGTG
GCGCTAGCGATATTGTGATGACTCAGTCTCCAGACTCCCTGGCTGTGTCTCTGGGCGA
GAGGGCCACCATGAACTGCAAGTCCAGCCATAGTGTTTTATACAGCTCCAACAATAAGA
ACTACTTAGCTTGGTACCAGCAGAAACCAGGACAGCCTCCTAAGCTGCTCATTTACTGG
GCATCTACCCGGGAATCCGGGGTCCCTGACCGATTCAGTGGCAGCGGGTCTGGGACA
GATTTCACTCTCACCATCAGCAGCCTGCAGCCTGAAGATGTGGCAGTTTATTTCTGTCA
GCAGTATTATAGTATCCCATTCACTTTCGGCCCGGGGACCAAGCTGGAGATCAAACGT
1G10 AMINO ACID SEQUENCE (SEQ ID NO: 38)
QVQLVESGPTLVKPTQTLTLTCTFSGFSLSTSGVGVGWIRQPPGKALEWLALIYRNEDKRY
SPSLERRLTITKDTSKNQVALTMTDMAPEDTATYYCAHRAATAAVLDYWGQGTLVTVSSL
EGGGGSGGGGSGGGASDIVMTQSPDSLAVSLGERATMNCKSSHSVLYSSNNKNYLAWY
QQKPGQPPKLLIYWASTRESGVPDRFSGSGSGTDFTLTISSLQPEDVAVYFCQQYYSIPFT
FGPGTKLEIKR
1H7 NUCLEOTIDE SEQUENCE (SEQ ID NO: 39)
GAGGTGCAGCTGTTGGAGTCTGGGGGAGGCGTGGTCCAGCCTGGGAGGTCCCTGAGA
CTCTCCTGTGCAGCCTCTGGATTCACCTTCAGTAGCTATGGCATGCACTGGGTCCGCC
AGGCTCCAGGCAAGGGGCTGGAGTGGGTGGCAGTTATATCATATGATGGAAGTAATAA
ATACTATGCAGACTCCGTGAAGGGCCGATTCACCATCTCCAGAGACAATTCCAAGAACA
CGCTGTATCTGCAAATGAACAGCCTGAGAGCTGAGGACACGGCTGTGTATTACTGTGC
GAAGCTATTAGGGTATAGCAGTGGCTGGTACCGTCCGGGGGCTTTTGATATCTGGGGC
CAAGGGACAATGGTCACCGTCTCTTCACTCGAGGGTGGAGGCGGTTCAGGCGGAGGT
GGCTCTGGCGGTGGCGCTAGCCAGTCTGCGCTGACTCAGCCTGCCTCCGTGTCTGGG
TCTCCTGGACAGTCGATCACCATCTCCTGCACTGGAACCAGCAGTGACGTTGGTAGAT
ATAACTATGTCTCCTGGTACCAACAACACCCAGGCAAAGCCCCCAAACTCATGATTTAT
GATGTCAGTAATCGGCCCTCAGGTGTTTCTAATCGCTTCTCTGGCTCCAAGTCTGGCAA
CACGGCCTCCCTGACCATCTCTGGGCTCCAGGCTGAGGACGAGGCTGATTATTACTGC
AGCTCATATACAAGCAGCAGCACTCGGGTGTTCGGCGGAGGGACCAAGGTCACCGTC
CTAGGT
1H7 AMINO ACID SEQUENCE (SEQ ID NO: 40)
EVQLLESGGGVVQPGRSLRLSCAASGFTFSSYGMHWVRQAPGKGLEWVAVISYDGSNKY
YADSVKGRFTISRDNSKNTLYLQMNSLRAEDTAVYYCAKLLGYSSGWYRPGAFDIWGQGT
MVTVSSLEGGGGGGGGSGGGASQSALTQPASVSGSPGQSITISCTGTSSDVGRYNYVS
WYQQHPGKAPKLMIYDVSNRPSGVSNRFSGSKSGNTASLTISGLQAEDEADYYCSSYTSS
STRVFGGGTKVTVLG
2B1 NUCLEOTIDE SEQUENCE (SEQ ID NO: 41)
CAGGTGCAGCTGGTGGAGTCTGGGGCTGAGGTGAAGAAGCCTGGGGCCTCAGTGAAG
GTCTCCTGCAAGGCTTCTGGATACACCTTCACCGGCTACTATATGCACTGGGTGCGAC
AGGCCCCTGGACAAGGGCTTGAGTGGATGGGACGGATCAACCCTAACAGTGGTGGCA
CAAACTATGCACAGAAGTTTCAGGGCAGGGTCACCATGACCAGGGACACGTCCATCAG
CACAGCCTACATGGAGCTGAGCAGGCTGAGATCTGACGACACGGCCGTGTATTACTGT
GCGAGAGTGGATTACGATATTTTGACTGGTTATTATCCCCCCCAGGACATGGACGTCTG
GGGCAAAGGGACAATGGTCACCGTCTCTTCACTCGAGGGTGGAGGCGGTTCAGGCGG
AGGTGGCTCTGGCGGTGGCGCTAGCGAAACGACACTCACGCAGTCTCCAGCATTCATG
TCAGCGACTCCAGGAGACAAAGTCAACATCTCCTGCAAAGCCAGCCAAGACATTGATG
ATGATATGAACTGGTACCAACAGAAACCAGGAGAAGCTCCTATTTTCATTATTCAAGAAG
CTACTACTCTCGTTCCTGGAATCCCACCTCGATTCAGTGGCAGCGGGTATGGAACAGAT
TTTACCCTCACAATTAATAACATAGAATCTGACGATGCTGCATATTACTTCTGTCTACAA
CATGATAATTTCCCGTACACTTTTGGCCAGGGGACCAAGGTGGAAATCAAACGT
2B1 AMINO ACID SEQUENCE (SEQ ID NO: 42)
QVQLVESGAEVKKPGASVKVSCKASGYTFTGYYMHWVRQAPGQGLEWMGRINPNSGGT
NYAQKFQGRVTMTRDTSISTAYMELSRLRSDDTAVYYCARVDYDILTGYYPPQDMDVWGK
GTMVTVSSLEGGGGSGGGGSGGGASETTLTQSPAFMSATPGDKVNISCKASQDIDDDMN
WYQQKPGEAPIFIIQEATTLVPGIPPRFSGSGYGTDFTLTINNIESDDAAYYFCLQHDNFPYT
FGQGTKVEIKR
2C8 NUCLEOTIDE SEQUENCE (SEQ ID NO: 43)
CAAGTGCAGCTGGTTGAATCTGGGGGAAGCGTGGTCCACCCGGGGAGGTCCCTGAGA
CTCTCCTGTGCGGCCTCTGGATTCACCTTCCGTAGCTATGCTATGCACTGGGTCCGCC
AGGCTCCAGGCAAGGGGCTGGAGTGGGTGGCAGTTATATCATATGATGGAAGTAATAA
ATACTACGCAGACTCCGTGAAGGGCCGATTCACCATCTCCAGAGACAATTCCAAGAACA
CGCTGTATCTGCAAATGAACAGCCTGAGAGCTGAGGACACGGCTGTGTATTACTGTGC
GAGATTAGCGACTACGGTGACTACGCTGAATGCTTTTGATATCTGGGGCCAAGGCACC
CTGGTCACCGTCTCCTCACTCGAGGGTGGAGGCGGTTCAGGCGGAGGTGGCTCTGGC
GGTGGCGCTAGCGACATCCAGATGACCCAGTCTCCTTCCACCCTGTCTGCATCTGTAG
GAGACAGAGTCACCATCACTTGCCGGGCAAGTCAGAGCATTAGCAGCTATTTAAATTGG
TATCAGCAGAAACCAGGGAAAGCCCCTAAGCTCCTGATCTATGCTGCATCCAGTTTGCA
AAGTGGGGTCCCATCAAGGTTCAGTGGCAGTGGATCTGGGGCAGATTTCACTCTCACC
ATCAGCAGCCTGCAGCCTGAAGATTTTGCAACTTATTACTGTCAACAGTTTAATAGTTAC
CCTCGAACGTTCGGCCAAGGGACCAAGCTGGAGATCAAACGT
2C8 AMINO ACID SEQUENCE (SEQ ID NO: 44)
QVQLVESGGSVVHPGRSLRLSCAASGFTFRSYAMHWVRQAPGKGLEWVAVISYDGSNKY
YADSVKGRFTISRDNSKNTLYLQMNSLRAEDTAVYYCARLATTVTTLNAFDIWGQGTLVTV
SSLEGGGGSGGGGSGGGASDIQMTQSPSTLSASVGDRVTITCRASQSISSYLNWYQQKP
GKAPKLLIYAASSLQSGVPSRFSGSGSGADFTLTISSLQPEDFATYYCQQFNSYPRTFGQG
TKLEIKR
2D3 NUCLEOTIDE SEQUENCE (SEQ ID NO: 45)
CAGGTCCAGCTTGTGCAGTCTGGGGCTGAGGTGAAGAAGCCTGGGGCCTCAGTGAAG
GTTTCCTGCAAGGCTTCTGGATACACCTTCACTAGCTATGCTATGCATTGGGTGCGCCA
GGCCCCCGGACAAAGGCTTGAGTGGATGGGATGGATCAACGCTGGCAATGGTAACAC
AAAATATTCACAGAAGTTCCAGGGCAGAGTCACCATTACCAGGGACACATCCGCGAGC
ACAGCCTACATGGAGCTGAGCAGCCTGAGATCTGAAGACACGGCTGTGTATTACTGTG
CGAGGGGCAGAATAGCAGCTCGTCGGGGGGACTACTACTACTACGGTATGGACGTCT
GGGGCCAAGGAACCCTGGTCACCGTCTCCTCACTCGAGGGTGGAGGCGGTTCAGGCG
GAGGTGGCTCTGGCGGTGGCGCTAGCAATTTTATGCTGACTCAGCCGCACTCTGTGTC
GGAGTCTCCGGGGAAGACGGTAATCATCTCCTGCACCCGCAGCAGTGGCAACATTGCC
AGCAACTATGTGCAGTGGTACCGGCAGCGCCCGGGCAGTGCCCCCACCTCTGTGATC
TATGAGGATAACCAAAGACCCTCTGGGGTCCCTGATCGGTTCTCTGGCTCCATCGACA
GCTCCTCCAACTCTGCCTCCCTCACCATCTCTGGACTGCAGACTGAGGACGAGGCTGA
CTACTACTGTCAGTCTTATGATAGCAGCACCCGGGTGTTCGGCGGAGGGACCAAGGTC
ACCGTCCTNGGT
2D3 AMINO ACID SEQUENCE (SEQ ID NO: 46)
QVQLVQSGAEVKKPGASVKVSCKASGYTFTSYAMHWVRQAPGQRLEWMGWINAGNGNT
KYSQKFQGRVTITRDTSASTAYMELSSLRSEDTAVYYCARGRIAARRGDYYYYGMDVWG
QGTLVTVSSLEGGGGSGGGGSGGGASNFMLTQPHSVSESPGKTVIISCTRSSGNIASNYV
QWYRQRPGSAPTSVIYEDNQRPSGVPDRFSGSIDSSSNSASLTISGLQTEDEADYYCQSY
DSSTRVFGGGTKVTVLG
2F1 NUCLEOTIDE SEQUENCE (SEQ ID NO: 47)
CAAGTTCAGCTGTTGCAGTCTGGAGCTGAGGTGAAGAAGCCTGGGGCCTCAGTGAAG
GTCTCCTGCAAGGCTTCTGGTTACACCTTTACCAGCTACGGTATCAGCTGGGTGCGAC
AGGCCCCTGGACAAGGGCTTGAGTGGATGGGATGGATCAGCGCTTACAATGGTAACAC
AAACTATGCACAGAAGCTCCAGGGCAGAGTCACGATTACCGCGGACAAATCCACGAGC
ACAGCCTACATGGAGCTGAGCAGCCTGAGATCTGAGGACACGGCCGTGTATTACTGTG
CGAGAGAGGGTCGGGATATTGTAGTGGTGGTAGCTGCTACATGGGACTACTACTACTA
CGGTATGGACGTCTGGGGCCAAGGGACAATGGTCACCGTCTCTTCACTCGAGGGTGG
AGGCGGTTCAGGCGGAGGTGGCTCTGGCGGTGGCGCTAGCGACATCCAGATGACCCA
GTCTCCATCCTCCCTGTCTGCATCTGTAGGAGACAGAGTCACCATCACTTGCCGGGCA
AGTCAGAGCATTAGCAGCTATTTAAATTGGTATCAGCAGAAACCAGGGAAAGCCCCTAA
GCTCCTGATCTACGATGCATCCAATTTGGACACAGGGGTCCCATCAAGGTTCAGTGGA
AGTGGATCTGGGACAGACTTCACTCTCACCATCAGCAGTCTGCACCCTGAAGATTTTGC
AACTTACTACTGTCAAGAGACTCACAGTGTCCCTCCTTGCAATTTTGGCCNGGGGACCA
AGGTGGAGATCAAACGT
2F1 AMINO ACID SEQUENCE (SEQ ID NO: 48)
QVQLLQSGAEVKKPGASVKVSCKASGYTFTSYGISWVRQAPGQGLEWMGWISAYNGNTN
YAQKLQGRVTITADKSTSTAYMELSSLRSEDTAVYYCAREGRDIVVVVAATWDYYYYGMD
VWGQGTMVTVSSLEGGGGSGGGGSGGGASDIQMTQSPSSLSASVGDRVTITCRASQSIS
SYLNWYQQKPGKAPKLLIYDASNLDTGVPSRFSGSGSGTDFTLTISSLHPEDFATYYCQET
HSVPPCNFGXGTKVEIKR
2F11 NUCLEOTIDE SEQUENCE (SEQ ID NO: 49)
CAGGTGCAGCTGGTGGAGTCTGGGGGAGGCGTGGTCCAGCCTGGGAGGTCCCTGAG
ACTCTCCTGTGCAGCCTCTGGATTCACCTTCAGTAGCTATGCTATGCACTGGGTCCGCC
AGGCTCCAGGCAAGGGGCTAGAGTGGGTGGCAGTTATATCATATGATGGAAGTAATAA
ATACTACGCAGACTCCGTGAAGGGCCGATTCACCATCTCCAGAGACAATTCCAAGAACA
CGCTGTATCTGCAAATGAACAGCCTGAGAGCTGAGGACACGGCTGTGTATTACTGTGC
GAGAATGTATAACTGGAACCAGCGCGGCGGGATACATGATGCTTTTGATATCTGGGGC
CAAGGGACAATGGTCACCGTCTCTTCACTCGAGGGTGGAGGCGGTTCAGGCGGAGGT
GGCTCTGGCGGTGGCGCTAGCTCCTATGAGCTGACTCAGGACCCTGCTGTGTCTGTG
GCCTTGGGACAGACAGTCAGGATCACATGCCAAGGAGACAGCCTCAGAACCTATTATG
CAAGCTGGTACCAGCAGAAGCCAGGACAGGCCCCTAAACTTGTCATCTATGGTAAAAA
CAACCGGCCCTCACGGATCCCAGACCGATTCTCTGGCTCCACCTCAGGAAACACAGCT
TCTTTGACCATCACAGGGGCTCAGGCGGAAGATGAGGCTGACTATTACTGTAACTCCC
GGGACAGCAGTGTTAACCGTCGTGATGTGGTCTTCGGCGGAGGGACCAAGGTCACCG
TCCTAGGT
2F11 AMINO ACID SEQUENCE (SEQ ID NO: 50)
QVQLVESGGGVVQPGRSLRLSCAASGFTFSSYAMHWVRQAPGKGLEWVAVISYDGSNKY
YADSVKGRFTISRDNSKNTLYLQMNSLRAEDTAVYYCARMYNWNQRGGIHDAFDIWGQG
TMVTVSSLEGGGGSGGGGSGGGASSYELTQDPAVSVALGQTVRITCQGDSLRTYYASWY
QQKPGQAPKLVIYGKNNRPSRIPDRFSGSTSGNTASLTITGAQAEDEADYYCNSRDSSVNR
RDVVFGGGTKVTVLG
2H6 NUCLEOTIDE SEQUENCE (SEQ ID NO: 51)
GAAGTGCAGCTGGTGCAGTCTGGGGCTGAGGTGAAGAAGCCTGGGGCCTCAGTGAAG
GTCTCCTGCAAGGCTTCTGGATACACCTTCACCGACTACTATATACACTGGGTGCGACA
GGCCCCTGGACAAGGGCTTGAGTGGATGGGAATAATCAACCCTAGTGGTGGTAGCACA
AGCTACGCACAGAAGTTCCAGGGCAGAGTCACCATGACCAGGGACACGTCCACGAGC
ACAGTCTACATGGAGCTGAGCAGCCTGAGATCTGAGGACACGGCCGTGTATTACTGTG
CCCGGCCAGTAGTACCCCCCAACTACTACTACGGTATGGACGTCTGGGGCCAAGGCAC
CCTGGTCACCGTCTCCTCACTCGAGGGTGGAGGCGGTTCAGGCGGAGGTGGCTCTGG
CGGTGGCGCTAGCGAAACGACACTCACGCAGTCTCCAGCATTCATGTCAGCGACTCCA
GGAGACAAAGTCAACATCCCCTGCAAAGCCAGCCAAGACATTGAGGATGATATGAACT
GGTACCAACAGAAACCAGGAGAAGCTGCTATTTTCATTATTCAAGAAGCTACTACTCTC
GTTCCTGGAATCTCACCTCGATTCAGTGGCAGCGGGTATGGAACAGATTTTACCCTCAC
AATTAATAACATAGAATCCGAGGATGCTGCATATTACTTCTGTCTACAACATGATAATTT
CCTCCAGGGCCAGGGGACCAAGCTGGAGATCAAACGT
2H6 AMINO ACID SEQUENCE (SEQ ID NO: 52)
EVQLVQSGAEVKKPGASVKVSCKASGYTFTDYYIHWVRQAPGQGLEWMGIINPSGGSTSY
AQKFQGRVTMTRDTSTSTVYMELSSLRSEDTAVYYCARPVVPPNYYYGMDVWGQGTLVT
VSSLEGGGGSGGGGSGGGASETTLTQSPAFMSATPGDKVNIPCKASQDIEDDMNWYQQK
PGEAAIFIIQEATTLVPGISPRFSGSGYGTDFTLTINNIESEDAAYYFCLQHDNFLQGQGTKL
EIKR
2H7 NUCLEOTIDE SEQUENCE (SEQ ID NO: 53)
GAAGTGCAGCTGGTGGAGTCTGGGGGAGGCGTGGTCCAGCCTGGGAGGTCCCTGAG
ACTCTCCTGTGCAGCCTCTGGATTCACCTTCAGTAGCTATGGCATGCACTGGGTCCGC
CAGGCTCCAGGCAAGGGGCTGGAGTGGGTGGCAGTTATATCATATGATGGAAGTAATA
AATACTATGCAGACTCCGTGAAGGGCCGATTCACCATCTCCAGAGACAATTCCAAGAAC
ACGCTGTATCTGCAAATGAACAGCCTGAGAGCTGAGGACACGGCTGTGTATTACTGTG
CGAAATTCCCCTCGCGTGGTAGGCTATATGCTTTTGATATCTGGGGCCAAGGGACAAT
GGTCACCGTCTCTTCACTCGAGGGTGGAGGCGGTTCAGGCGGAGGTGGCTCTGGCGG
TGGCGCTAGCGACATCCAGATGACCCAGTCCCCCTCTTCCGTGTCTGCGTCTGCAGGA
GACAGAGTCACCATCACTTGTCGGGCGAGTCAGGATATTAGCAGCTGGTTAGCCTGGT
ATCGACAGGAACTAGGGAAACCCCCTAAACTCCTGATCTATGCTGCATCCAGTTTGCAA
AGGGGAGTCCCATCCAGGTTCAGCGGCAGTGGATCTGGGACAGATTTCACTCTCACCA
TCAGCAGCCTGCAGCCTGAAGATTTTGGAATCTACTACTGTCAACAGTCTAACAGTTTC
CCGTACACCTTCGGCCAAGGGACACGACTGGAGATTAAACGT
2H7 AMINO ACID SEQUENCE (SEQ ID NO: 54)
EVQLVESGGGVVQPGRSLRLSCAASGFTFSSYGMHWVRQAPGKGLEWVAVISYDGSNKY
YADSVKGRFTISRDNSKNTLYLQMNSLRAEDTAVYYCAKFPSRGRLYAFDIWGQGTMVTV
SSLEGGGGSGGGGSGGGASDIQMTQSPSSVSASAGDRVTITCRASQDISSWLAWYRQEL
GKPPKLLIYAASSLQRGVPSRFSGSGSGTDFTLTISSLQPEDFGIYYCQQSNSFPYTFGQGT
RLEIKR
2H9 NUCLEOTIDE SEQUENCE (SEQ ID NO: 55)
CAGGTGCAGCTGGTGGAGTCTGGGGGAGGCTTGGTACAGCCTGGAGGGTCCCTGAGA
CTCTCCTGTGCAGCCTCTGGATTCACCTTCAGTAGTTATGAAATGAACTGGGTCCGCCA
GGCTCCAGGGAAGGGGCTTGAGTGGGTTTCATTCATTACTAGTCGTGATAATACTATAT
ACTACGCAGACTCTGTGAAGGGCCGATTCACCATCTCCAGAGACAATTCCAAGAACAC
GCTGTATCTGCAAATGGACAGTCTGAGAGCCGAGGACACGGCTGTGTATTATTGTACAA
GAGTCTTAAATGGCCTAAGCGGACACTTTGACCACTGGGGCCAGGGAACCCTGGTCAC
CGTCTCCTCACTCGAGGGTGGAGGCGGTTCAGGCGGAGGTGGCTCTGGCGGTGGCG
CTAGCAATTTTATGCTGACTCAGCCCCACTCTGTGTCGGGTTCTCCGGGGAAAACGGT
CACCATATCCTGCACCGGCAGCAGTGGCAGCATTGCCAGGAACTATGTGCAGTGGTAC
CAGCAGCGCCCGGGCAGTGCCCCCACCACTGTGATCTATGAGGATAATCAGAGACCCT
CTGGGGTCCCTGATCGGTTCTCTGGCTCCATCGACAGCTCCTCCAACTCTGCCTCCCT
CACCATCTCTGGACTGAAGACTGAGGACGAGGCTGACTACTACTGTCAGTCTTATGATA
CCAGCATTCATTATGTCTTCGGAACTGGGACCAAGCTGACCGTCCTAGGT
2H9 AMINO ACID SEQUENCE (SEQ ID NO: 56)
QVQLVESGGGLVQPGGSLRLSCAASGFTFSSYEMNWVRQAPGKGLEWVSFITSRDNTIYY
ADSVKGRFTISRDNSKNTLYLQMDSLRAEDTAVYYCTRVLNGLSGHFDHWGQGTLVTVSS
LEGGGGSGGGGSGGGASNFMLTQPHSVSGSPGKTVTISCTGSSGSIARNYVQWYQQRP
GSAPTTVIYEDNQRPSGVPDRFSGSIDSSSNSASLTISGLKTEDEADYYCQSYDTSIHYVFG
TGTKLTVLG
3A5 NUCLEOTIDE SEQUENCE (SEQ ID NO: 57)
GAAGTGCAGCTGGTGGAGTCTGGGGGAGGCGTGGTCCAGCCTGGGAGGTCCCTGAG
GCTCTCCTGTGCAGCCTCTGGATTCACCTTCAGTAGCTATGCTATGCACTGGGTCCGC
CAGGCTCCAGGCAAGGGGCTGGAGTGGGTGGCAGTTATATCATATGATGGAAGCAATA
AATACTACGCAGACTCCGTGAAGGGCCGATTCACCATCTCCAGAGACAATTCCAAGAAC
ACGCTGTATCTGCAAATGAACAGCCTGAGAGCTGAGGACACGGCTGTGTATTACTGTG
CCCGGATAGCAGTGGCTGGTCGATCCCAAAATGTTGACTACTGGGGCCAGGGAACCCT
GGTCACCGTCTCCTCACTCGAGGGTGGAGGCGGTTCAGGCGGAGGTGGCTCTGGCGG
TGGCGCTAGCTCCTATGAGCTGACTCAGGACCCTGCTGTGTCTGTGGCCTTGGGACAG
ACAGTCAGGATCACATGCCAAGGAGACAGCCTCAGAACCTATTATGGAAGTTGGTACC
AACAGAAGCCAGGACAGGCCCCTGCCCTTGTCATGTATGGTAGAGACACCCGGCCCTC
AGGAATCCCAGACCGATTCTCTGCCTCCAGTTCGAGCAACACAGCTTCCTTGACCATCA
CTGGGGCTCAGGCGGAAGATGAGGCTGACTATTGGTGTAGTTCCCGGGACACCAGTG
TTAAAAATGGTGTGGTTTTCGGCGGAGGGACCAAGGTCACCGTCCTAGGT
3A5 AMINO ACID SEQUENCE (SEQ ID NO: 58)
EVQLVESGGGVVQPGRSLRLSCAASGFTFSSYAMHWVRQAPGKGLEWVAVISYDGSNKY
YADSVKGRFTISRDNSKNTLYLQMNSLRAEDTAVYYCARIAVAGRSQNVDYWGQGTLVTV
SSLEGGGGSGGGGSGGGASSYELTQDPAVSVALGQTVRITCQGDSLRTYYGSWYQQKP
GQAPALVMYGRDTRPSGIPDRFSASSSSNTASLTITGAQAEDEADYWCSSRDTSVKNGVV
FGGGTKVTVLG
3A12 NUCLEOTIDE SEQUENCE (SEQ ID NO: 59)
CAGGTGCAGCTGGTGGAGTCTGGGGGAGGCGTGGTCCAGCCTGGGAGGTCCCTGAG
ACTCTCCTGTGCAGCCTCTGGATTCACCTTCAGTAGCTATGGCATGCACTGGGTCCGC
CAGGCTCCAGGCAAGGGGCTGGAGTGGGTGGCAGTTATATCATATGATGGAAGTAATA
AATACTACGCAGACTCCGTGAAGGGCCGATTCACCATCTCCAGAGACAACGCCAAGAA
CACGCTGTATCTGCAAATGAACAGTCTGAGAGCCGAGGACACGGCTGTATATTACTGT
GCAAGAGTCGGGGGAAGGGATTCTTTTGATATCTGGGGCCAAGGAACCCTGGTCACCG
TCTCCTCACTCGAGGGTGGAGGCGGTTCAGGCGGAGGTGGCTCTGGCGGTGGCGCTA
GCGATATTGTGATGACTCAGTCTCCACTCTCCCTGCCCGTCACCCTTGGACAGCCGGC
CTCCATCTCCTGCAGGTCTAGTCAGAGCCTCGTATACAGTGATGGAAACACCTACTTGA
ATTGGTTTCAGCAGAGGCCAGGCCAATCTCCAAGGCGCCTAATTTATAAGGTTTCTATT
CGGGACTCTGGGGTCCCAGACAGATTCAGCGGCAGTGGGTCAGGCACTGATTTCACA
CTGAAAATCAGCAGGGTGGAGGCTGAGGATGTTGGGGTTTATTACTGCATGCAATCTA
CACACTGGCCTCCGACGTTCGGCCAAGGGACCAAGGTGGAAATCAAACGT
3A12 AMINO ACID SEQUENCE (SEQ ID NO: 60)
QVQLVESGGGVVQPGRSLRLSCAASGFTFSSYGMHWVRQAPGKGLEWVAVISYDGSNKY
YADSVKGRFTISRDNAKNTLYLQMNSLRAEDTAVYYCARVGGRDSFDIWGQGTLVTVSSL
EGGGGSGGGGSGGGASDIVMTQSPLSLPVTLGQPASISCRSSQSLVYSDGNTYLNWFQQ
RPGQSPRRLIYKVSIRDSGVPDRFSGSGSGTDFTLKISRVEAEDVGVYYCMQSTHWPPTF
GQGTKVEIKR
3B4 NUCLEOTIDE SEQUENCE (SEQ ID NO: 61)
CAGGTGCAGCTGGTGGAGTCTGGGGGAGGCGTGGTCCAGCCTGGGAGGTCCCTGAG
ACTCTCCTGTGCAGCCTCTGGATTCACCTTCAGTAGCTATGGCATGCACTGGGTCCGC
CAGGCTCCAGGCAAGGGGCTGGAGTGGGTGGCAGTTATATCATATGATGGAAGTAATA
AATACTATGCAGACTCCGTGAAGGGCCGATTCACCATCTCCAGAGACAATTCCAAGAAC
ACGCTGTATCTGCAAATGAACAGCCTGAGAGCTGAGGACACGGCTGTGTATTACTGTG
CGAAATTAGTGGGTAACTGGAACTTTTACGACTACTGGGGCCAGGGCACCCTGGTCAC
CGTCTCCTCACTCGAGGGTGGAGGCGGTTCAGGCGGAGGTGGCTCTGGCGGTGGCG
CTAGCGATATTGTGATGACCCAGTCTCCAGCCGCCCTGTCTGTGTCTCCAGGGGAAAG
AGCCACCCTCTCCTGCAGGGCCAGTCAGAGTGTTAGCAGCAACTTAGCCTGGTACCAG
CAGAAACCTGGCCAGGCTCCCAGGCTCCTCATCTATGGTGCATCCACCAGGGCCACTG
GTATCCCAGCCAGGTTCAGTGGCAGTGGGTCTGGGACAGAGTTCACTCTCACCATCAG
CAGCCTGCAGTCTGAAGATTTTGCAGTTTACTACTGTCAGCAGTATAATAACTGGCCCA
GGACGTTCGGCCAAGGGACCAAGGTGGAAATCAAACGT
3B4 AMINO ACID SEQUENCE (SEQ ID NO: 62)
QVQLVESGGGVVQPGRSLRLSCAASGFTFSSYGMHWVRQAPGKGLEWVAVISYDGSNKY
YADSVKGRFTISRDNSKNTLYLQMNSLRAEDTAVYYCAKLVGNWNFYDYWGQGTLVTVSS
LEGGGGSGGGGSGGGASDIVMTQSPAALSVSPGERATLSCRASQSVSSNLAWYQQKPG
QAPRLLIYGASTRATGIPARFSGSGSGTEFTLTISSLQSEDFAVYYCQQYNNWPRTFGQGT
KVEIKR
3B11 NUCLEOTIDE SEQUENCE (SEQ ID NO: 63)
CAGGTGCAGCTGGTGGAGTCTGGGGGAGGCGTGGTCCAGCCTGGGAGGTCCCTGAG
ACTCTCCTGTGCAGCGTCTGGATTCACCTTCAGTAGCTATGGCATGCACTGGGTCCGC
CAGGCTCCAGGCAAGGGGCTGGAGTGGGTGGCAGTTATATCATATGATGGAAGTAATA
AATACTACGCAGACTCCGTGAAGGGCCGATTCACCATCTCCAGAGACAATTCCAAGAAC
ACGCTGTATCTGCAAATGAACAGCCTGAGAGCTGAGGACACGGCTGTGTATTACTGTG
CGAGAATAGCAGTGGCTGGCTCATCACAAGCGTCCTACTTTGACTACTGGGGCCAGGG
AACCCTGGTCACCGTCTCCTCACTCGAGGGTGGAGGCGGTTCAGGCGGAGGTGGCTC
TGGCGGTGGCGCTAGCGATATTGTGATGACCCAGTCTCCACTCTCCCTGCCCGTCACC
CTTGGACAGCCGGCCTCCATCTCCTGCAGGTCTAGTCAAAGCCTCGTATACAGTGATG
GAAACACCTACTTGAATTGGTTTCAGCAGAGGCCAGGCCAATCTCCAAGGCGCCTAATT
TATAAGGTTTCTAACCGGGACTCTGGGGTCCCAGACAGATTCACCGGCAGTGGGTCAG
GCACTGATTTCACACTGAAAATCAGCAGGGTGGAGGCTGAAGATGTTGGGGTTTATTAC
TGCATGCAAGGTACACACTGGCCCCCAACGTTCGGCCAAGGGACCAAGCTGGAGATCA
AACGT
3B11 AMINO ACID SEQUENCE (SEQ ID NO: 64)
QVQLVESGGGVVQPGRSLRLSCAASGFTFSSYGMHWVRQAPGKGLEWVAVISYDGSNKY
YADSVKGRFTISRDNSKNTLYLQMNSLRAEDTAVYYCARIAVAGSSQASYFDYWGQGTLV
TVSSLEGGGGSGGGGSGGGASDIVMTQSPLSLPVTLGQPASISCRSSQSLVYSDGNTYLN
WFQQRPGQSPRRLIYKVSNRDSGVPDRFTGSGSGTDFTLKISRVEAEDVGVYYCMQGTH
WPPTFGQGTKLEIKR
3C4 NUCLEOTIDE SEQUENCE (SEQ ID NO: 65)
GAAGTGCAGCTGGTGCAGTCTGGGGCTGAGGTGAAGAAGCCTGGGGCCTCAGTGAAG
GTTTCCTGCAAGGCATCTGGATACACCTTCACCAGCTACTATATGCACTGGGTGCGACA
GGCCCCTGGACAAGGGCTTGAGTGGATGGGAATAATCAACCCTAGTGGTGGTAGCACA
AGCTACGCACAGAAGTTCCAGGGCAGAGTCACCATGACCAGGGACACGTCCACGAGC
ACAGTCTACATGGAGCTGAGCAGCCTGAGATCTGAGGACACGGCCGTGTATTACTGTG
CGAGAGGCCCCCGAAGTGGCCGATGGGGGTACTGGGGCCAGGGAACCCTGGTCACC
GTCTCCTCACTCGAGGGTGGAGGCGGTTCAGGCGGAGGTGGCTCTGGCGGTGGCGCT
AGCGACATCCAGATGACCCAGTCTCCAGACTCCCTGGCTGTGTCTCTGGGCGAGAGG
GCCACCATCAACTGCAAGTCCAGCCAGAGTGTTTTATACACCTCCAACAATAAGAACTA
CTTAGCTTGGTACCAGCAGAAACCAGGACAGCCTCCTAAGCTGCTCATTTACTGGGCAT
CGACCCGGGAATCCGGGGTCCCTGACCGATTCAGTGGCAGCGGGTCTGGGACAGATT
TCACTCTCACCATCAGCAGCCTGCAGGCTGAAGATGTGGCAGTTTATCACTGTCAGCAA
TATTATAGTCTTCCTATCACCTTCGGCCAAGGGACACGACTGGAGATTAAACGT
3C4 AMINO ACID SEQUENCE (SEQ ID NO: 66)
EVQLVQSGAEVKKPGASVKVSCKASGYTFTSYYMHWVRQAPGQGLEWMGIINPSGGSTS
YAQKFQGRVTMTRDTSTSTVYMELSSLRSEDTAVYYCARGPRSGRWGYWGQGTLVTVSS
LEGGGGSGGGGSGGGASDIQMTQSPDSLAVSLGERATINCKSSQSVLYTSNNKNYLAWY
QQKPGQPPKLLIYWASTRESGVPDRFSGSGSGTDFTLTISSLQAEDVAVYHCQQYYSLPIT
FGQGTRLEIKR
3C10 NUCLEOTIDE SEQUENCE (SEQ ID NO: 67)
CAGGTGCAGCTGGTGGAGTCTGGGGGAGGCGTGGTCCAGCCTGGGAGGTCCCTGAG
ACTCTCCTGTGCAGCCTCTGGATTCACCTTCAGTAGCTATGGCATGCACTGGGTCCGC
CAGGCTCCAGGCAAGGGGCTGGAGTGGGTGGCAGTTATATCATATGATGGAAGTAATA
AATACCTTGCAGACTCCGTGAAGGGCCGATTCACCATCTCCAGAGACAATTCCAAGAAC
ACGCTGTATCTGCAAATGAACAGCCTGAGAGCTGAGGACACGGCTGTGTATTACTGTG
CGAAGTTGGCTAGATATTGTAGTGGTGGTAGGTGCCCGTACCACCACGGTATGGACGT
CTGGGGCCAAGGCACCCTGGTCACCGTCTCCTCACTCGAGGGTGGAGGCGGTTCAGG
CGGAGGTGGCTCTGGCGGTGGCGCTAGCGACATCCAGATGACCCAGTCTCCATCCTC
CCTGTCTGCATCTGTAGGAGACAGAGTCACCATCACTTGCCGGGCAAGTCAGAGCATT
AGCAGCTATTTAAATTGGTATCAGCAGAAACCAGGGAAAGCCCCTAAGCTCCTGATCTA
TGCTGCATCCACTTTGCAAAGTGGGGTCCCATCAAGGTTCAGTGGCAGTGGATCTGGG
ACAGATTTCACTCTCACCATCAGCAGTCTGCAACCTGAAGATTTTGCAACTTACTACTGT
CAACAGAGTTACAGTACCCCGATCACCTTCGGCCAAGGGACACGACTGGAGATCAAAC
GT
3C10 AMINO ACID SEQUENCE (SEQ ID NO: 68)
QVQLVESGGGVVQPGRSLRLSCAASGFTFSSYGMHWVRQAPGKGLEWVAVISYDGSNKY
LADSVKGRFTISRDNSKNTLYLQMNSLRAEDTAVYYCAKLARYCSGGRCPYHHGMDVWG
QGTLVTVSSLEGGGGSGGGGSGGGASDIQMTQSPSSLSASVGDRVTITCRASQSISSYLN
WYQQKPGKAPKLLIYAASTLQSGVPSRFSGSGSGTDFTLTISSLQPEDFATYYCQQSYSTPI
TFGQGTRLEIKR
3D2 NUCLEOTIDE SEQUENCE (SEQ ID NO: 69)
GAAGTGCAGCTGGTGGAGTCAGGGGGTGGCGTGGTCCAGCCTGGGAGGTCCCTGAG
ACTCTCCTGTGCAGCGTCTGGATTCACCTTCAGTAGCTATGGCATGCACTGGGTCCGC
CAGGCTCCAGGCAAGGGGCTGGAGTGGGTGGCAGTTATATCATATGATGGAAGTAATA
AATACTATGCAGACTCCGTGAAGGGCCGATTCACCATCTCCAGAGACAATTCCAAGAAC
ACGCTGTATCTGCAAATGAACAGCCTGAGAGCTGAGGACACGGCTGTGTATTACTGTG
CGAGAACCGGGCGATCTGCTTTTGATATCTGGGGCCAAGGGACAATGGTCACCGTCTC
TTCACTCGAGGGTGGAGGCGGTTCAGGCGGAGGTGGCTCTGGCGGTGGCGCTAGCG
ATATTGTGATGACCCAGTCTCCACTCTCCCTGCCCGTCACCCTTGGACAGCCGGCCTC
CATCTCCTGCAGGTCTAGTCAAAGCCTCGTATACAGTGATGGAAACACCTACTTGAATT
GGTTTCAGCAGAGGCCAGGCCAATCTCCAAGGCGCCTAATTTATAAGGTTTCTAACCG
GGACTCTGGGGTCCCAGACAGATTCAGCGGCAGTGGGTCAGGCACTTATTTCACACTG
GAAATCAGCAGGGTGGAGGCTGAGGATGTTGGAGTTTATTACTGCATGCAAGGTACAC
ACTGGCCGCTCACTTTCGGCGGAGGGACCAAAGTGGATATCAAACGT
3D2 AMINO ACID SEQUENCE (SEQ ID NO: 70)
EVQLVESGGGVVQPGRSLRLSCAASGFTFSSYGMHWVRQAPGKGLEWVAVISYDGSNKY
YADSVKGRFTISRDNSKNTLYLQMNSLRAEDTAVYYCARTGRSAFDIWGQGTMVTVSSLE
GGGGSGGGGSGGGASDIVMTQSPLSLPVTLGQPASISCRSSQSLVYSDGNTYLNWFQQR
PGQSPRRLIYKVSNRDSGVPDRFSGSGSGTYFTLEISRVEAEDVGVYYCMQGTHWPLTFG
GGTKVDIKR
3D6 NUCLEOTIDE SEQUENCE (SEQ ID NO: 71)
CAGGTGCAGCTGGTGGAGTCTGGGGGAGGCGTGGTCCAGCCTGGGAGGTCCCTGAG
ACTCTCCTGTGCAGCGTCTGGATTCAGCTTCAGTAACTATGGCATGCACTGGGTCCGC
CAGGCTCCAGGCAAGGGGCTGGAGTGGGTGGCAGTTATATCATATGATGGAAGTAAGG
AATACTATGTAGACTCCGTGAAGGGCCGATTCACCATCTTCAGAGACAATTCCAAGAAC
ACCCTGTATCTGCAAATGAACAGCCTGAGAGCCGAGGACACGGCTGTGTATTACTGTG
CGAAACTAGCAATTGGGTCACCAGGTGACGACGACTACTGGGGCCAGGGAACCCTGG
TCACCGTCTCCTCACTCGAGGGTGGAGGCGGTTCAGGCGGAGGTGGCTCTGGCGGTG
GCGCTAGCCAGTCTGAGCTGACTCAGCCTCCCTCCGCGTCCGGGTCTCCTGGACAGT
CAGTCACCATCTCCTGCACTGGAACCAGCAGTGACGTTGGTGGTTATAACTATGTCTCC
TGGTACCAACAGCACCCAGGCAAAGCCCCCAAACTCATGATTTATGAGGTCAGTAGGC
GGCCCTCAGGGGTCCCTGATCGCTTCTCTGGCTCCAAGTCTGGCAACACGGCCTCCCT
GACCGTCTCTGGGCTCCAGGCTGAGGATGAGGCTGATTATTACTGCAGCTCATATGCA
GGCAGCAACACCGTGGTATTCGGCGGAGGGACCCAGCTCACCGTTTTAGGT
3D6 AMINO ACID SEQUENCE (SEQ ID NO: 72)
QVQLVESGGGVVQPGRSLRLSCAASGFSFSNYGMHWVRQAPGKGLEWVAVISYDGSKEY
YVDSVKGRFTIFRDNSKNTLYLQMNSLRAEDTAVYYCAKLAIGSPGDDDYWGQGTLVTVS
SLEGGGGSGGGGSGGGASQSELTQPPSASGSPGQSVTISCTGTSSDVGGYNYVSWYQQ
HPGKAPKLMIYEVSRRPSGVPDRFSGSKSGNTASLTVSGLQAEDEADYYCSSYAGSNTVV
FGGGTQLTVLG
3H8 NUCLEOTIDE SEQUENCE (SEQ ID NO: 73)
CAGGTGCAGCTGGTGGAGTCTGGGGGAGGCGAGGTCCAGCCAGGGAGGTCCCTGAG
ACTCTCCTGTGCAGCCTCTGGATTCACCTTCAGTAGCTATGGCAGGCACTGGGTCCGC
CAGGCTCCAGGCAAGGGGCTGGAGTGGGTGGCAGTTATATCATATGATGGAAGTAATA
AATACTATGCAGACTCCGTGAAGGGCCGATTCACCATCTCCAGAGACAATTCCAAGAAC
ACGCTGTATCTGCAAATGAACAGCCTGAGAGCTGAGGACACGGCTGTGTATTACTGTG
CGAGACTTTACTATGGTTCGGGGGTGCTGGGGAACGGTATGGACGTCTGGGGCCAAG
GCACCCTGGTCACCGTCTCCTCACTCGAGGGTGGAGGCGGTTCAGGCGGAGGTGGCT
CTGGCGGTGGCGCTAGCCAGTCTGTGCTGACTCAGCCACCCTCAGCGTCTGGGACCC
CCGGGCAGAGGGTCACCATCTCTTGTTCTGGAAGCAGCTCCGACATCGGAAGTAATAC
TGTAAACTGGTACCAGCAACTCCCAGGAACGGCCCCCAAACTCCTCATCTACAATAATA
ATCAGCGGCCCTCAGGGGTCCCTGACCGATTCTCTGGCTCCAAAGATGCTTCGGCCAA
TGCAGGGATTTTACTCATCTCTGGGCTCCAGTCTGACGATGAGGGTGACTATTATTGTA
TGATTTGGCACAGCAGCGCTTGGGTGTTCGGCGGAGGGACCCAGCTCACCGTTTTAGG
T
3H8 AMINO ACID SEQUENCE (SEQ ID NO: 74)
QVQLVESGGGEVQPGRSLRLSCAASGFTFSSYGRHWVRQAPGKGLEWVAVISYDGSNKY
YADSVKGRFTISRDNSKNTLYLQMNSLRAEDTAVYYCARLYYGSGVLGNGMDVWGQGTL
VTVSSLEGGGGSGGGGSGGGASQSVLTQPPSASGTPGQRVTISCSGSSSDIGSNTVNWY
QQLPGTAPKLLIYNNNQRPSGVPDRFSGSKDASANAGILLISGLQSDDEGDYYCMIWHSSA
WVFGGGTQLTVLG
3H11 NUCLEOTIDE SEQUENCE (SEQ ID NO: 75)
GAGGTGCAGCTGGTGGAGTCTGGGGGAGGCGTGGTCCAGCCTGGGAGGTCCCTGAG
ACTCTCCTGTGCAGCCTCTGGATTCACCTTCAGTAGCTATGGCATACACTGGGTCCGCC
AGGCTCCAGGCAAGGGGCTGGAGTGGGTGGCAGTTATATCATATGATGGAAGTAATAA
ATACTACGCAGACTCCGTGAAGGGCCGATTCACCATCTCCAGAGACAATTCCAAGAACA
CGCTGTATCTGCAAATGAACAGCCTGAGAGCTGAGGACACGGCTGTGTATTACTGTGC
GAGATACCCTACCAGCGGTAGTAGTTATTACGTGAATGACTACTGGGGCCAGGGCACC
CTGGTCACCGTCTCCTCACTCGAGGGTGGAGGCGGTTCAGGCGGAGGTGGCTCTGGC
GGTGGCGCTAGCGACATCCAGATGACCCAGTCTCCATCCTTCCTGTCTGCATCTGTAG
GAGACAGAGTCACCATCACTTGCCGGGCAAGTCAGAGCATTAGCAGCTATTTAAATTGG
TATCAGCAGAAACCAGGGAAAGCCCCTAAGCTCCTGATCTATGCTGCATCCAGGTTGC
AAAGTGAGGTCCCGTCAAGGTTCAGTGGCAGTGGATCTGGGACAGATTTCACTCTCAC
CATCAGCAGTCTGCAACCTGAAGACTTTGCAACTTACTACTGTCAACAGAGTTACAGAA
CCCCGCTCACTTTCGGCGGAGGGACCAAGGTGGAGATCAAACGT
3H11 AMINO ACID SEQUENCE (SEQ ID NO: 76)
EVQLVESGGGVVQPGRSLRLSCAASGFTFSSYGIHWVRQAPGKGLEWVAVISYDGSNKYY
ADSVKGRFTISRDNSKNTLYLQMNSLRAEDTAVYYCARYPTSGSSYYVNDYWGQGTLVTV
SSLEGGGGSGGGGSGGGASDIQMTQSPSFLSASVGDRVTITCRASQSISSYLNWYQQKP
GKAPKLLIYAASRLQSEVPSRFSGSGSGTDFTLTISSLQPEDFATYYCQQSYRTPLTFGGG
TKVEIKR
***
PGA31 PEPTIDE SEQUENCE (SEQ ID NO: 77)
QPLNVGNTVLQLGGSGDGTKVDIAEDGTLS
UTR2 PEPTIDE SEQUENCE (SEQ ID NO: 78)
WPGGDSSNAKGTIEWAGGLINWDSEDIK (SEQ ID NO: 78)
PHR2 PEPTIDE SEQUENCE (SEQ ID NO: 79)
QDAGIYVIADLSQPDESINRDDPSWDLDLFER
CHT2 PEPTIDE SEQUENCE (SEQ ID NO: 80)
LGKTVLLSLGGGVGDYGFSDVASATK
***
PGA31 AMINO ACID SEQUENCE (SEQ ID NO: 81)
MKFHMRLQKKIFVLEYYIKPDISSFSGKYLFLLFFLFQSHINQLFDYIYFIQKYLICYIMKFLTAA
SLLTLSSSALAAIKDIQLYAQSSNNEVNDFGISSRHEGAALNYLFLAAPGVAENLKYDDETKT
VYTELKAGSSTVRQPLNVGNTVLQLGGSGDGTKVDIAEDGTLSFDGSDSVGAAKNINDPYN
YSKDSYAVVKGGDGAIPIKLVAKFTGDDKESASSSSSSAAPEPTASSSEAPKETPVYSNSTV
TLYTTYCPLSTTITLTVCSDVCTPTVIETSGSVTVSSVQVPSKTASSEAAPPKTTVDSVSKPA
PSGKKPTAAVTSFEGAANALTGGSVAIAVAAAIGLVF
UTR2 AMINO ACID SEQUENCE (SEQ ID NO: 82)
MRFSTLHFAFLATLSSIFTWVAASDTTTCSSSKHCPEDKPCCSQFGICGTGAYCLGGCDIRY
SYNLTACMPMPRMSTFQESFDSKDKVKEIELQSDYLGNSTEADWVYTGWVDYYDNSLLIQ
MPNHTTGTVVSSTKYLWYGKVGATLKTSHDGGVVTAFILFSDVQDEIDYEFVGYNLTNPQS
NYYSQGILNYNNSRNSSVNNTFEYYHNYEMDWTEDKIEWYIDGEKVRTLNKNDTWNETSN
RYDYPQTPSRIQFSLWPGGDSSNAKGTIEWAGGLINWDSEDIKKYGYYYAHIKEIYATAYDI
PNDVKLDGNSTKESDYHAFLYNSTDGDASNIMLTTKKTWLGSDDATGFDPQNDDEDSSSN
KAQETTITSVSGSSTITSVKTDSTKKTANVPAQNTAAAAQATAKSSTGTNTYDPSAGVGGFV
QDSKSTDSGSSGSSSQGVANSLNESVISGIFASICLGILSFFM
PHR2 AMINO ACID SEQUENCE (SEQ ID NO: 83)
MLLKSLFPSILAATSFVSSVAAEDLPAIEIVGNKFFYSNNGSQFYIKGIAYQQNNLDSNESFVD
PLANPEHCKRDIPYLEAVDTNVIRVYALDTSQDHTECMQMLQDAGIYVIADLSQPDESINRD
DPSWDLDLFERYTSVVDLFHNYTNILGFFAGNEVTNKKSNTDASAFVKAAIRDTKAYIKSKG
YRSIPVGYSANDDSAIRVSLADYFACGDEDEAADFFGINMYEWCGDSSYKASGYESATNDY
KNLGIPIFFSEYGCNEVRPRKFTEVATLFGDQMTPVWSGGIVYMYFEEENNYGLVSIKDNTV
STLKDYSYYSSEIKDIHPSSAKASAESASSISRTTCPTNTNNWEASTNLPPTPDKEVCECMS
ASLKCVVDDKVDSDDYSDLFSYICAKIDCDGINANGTTGEYGAYSPCHSKDKLSFVMNLYY
EQNKESKSACDFGGSASLQSAKTASSCSAYLSSAGSSGLGTVSGTVRTDTSQSTSDSGSG
SSSSSSSSSSSSSSGSSGSKSAASIVSVNLLTKIATIGISIVVGFGLITM
CHT2 AMINO ACID SEQUENCE (SEQ ID NO: 84)
MLSFKSLLAAAVVASSALASASNQVALYWGQNGAGGQERLAQYCQETDVDIVLLSFLNLFP
DPLNVNFANQCGNTFESGLLHCSQIGADIKTCQSLGKTVLLSLGGGVGDYGFSDVASATKF
ADTLWNKFGAGEDPERPFDDAVVDGFDFDIEHGGATGYPELATALRGKFAKDTSKNYFLS
AAPQCPYPDASLGDLLSKVPLDFAFIQFYNNYCSINGQFNYDTWSKFADSAPNKNIKLFVGV
PATSNIAGYVDTSKLSSAIEEIKCDSHFAGVSLWDASGAWLNTDEKGENFVVQVKNVLNQN
ACVAPSSSATTQSTTTTSSAVTQSTTTTSAAITQSATTTSAAVTTKSNQIVTSSSSSSSSIFY
GNSTTESSTGIATGfTVLPTGSNENAATTGSGSNTKLAISTVTDVQKTVITITSCSEHKCVATP
VTTGVVWVTDIDTVYTTYCPLTNSQVYVPVQTVVCTEETCVPSPTSTAQKPKASTTIKGVEK
GQTTSYPVVGTTEGVKKIVTTSAQTVGSSTKYVTIELTSTITPVTYPTSVASNGTNTTVPVFT
FEGGAAVANSLNSVWFPVPFLLAAFAF

1. A method for identifying epitopes of fungal cell wall proteins (“CWPs”) suitable for the development of antifungal antibodies, comprising:
(a) providing a population of a first strain of a fungal species;
(b) culturing:
(i) a first sub-population of the first strain in the presence of an antifungal agent, thereby forming a first culture;
(ii) a second sub-population of the first strain in the absence of an antifungal agent, thereby forming a second culture;
(c) harvesting the cells and isolating the cell walls from each culture;
(d) digesting the cell walls from each culture with a digesting agent for surface-exposed CWPs, thereby forming digested surface-exposed fungal CWPs;
(e) performing proteomic analysis on the digested surface-exposed fungal CWPs; and
(f) identifying surface-exposed epitopes of the digested surface-exposed CWPs that are expressed in higher abundance in the first culture compared to the second culture.

2. (canceled)

3. A method for identifying epitopes of fungal cell wall proteins (“CWPs”) suitable for the development of antifungal antibodies, comprising:
(a) providing populations of a first strain and a second strain of a fungal species, wherein the first strain is resistant to an antifungal drug, and the second strain is not resistant to an antifungal drug;
(b) culturing:
(i) a first sub-population of the first strain in the presence of an antifungal agent, thereby forming a first culture;
(ii) a second sub-population of the first strain in the absence of an antifungal agent, thereby forming a second culture;
(iii) a first sub-population of the second strain in the presence of an antifungal agent, thereby forming a third culture; and
(iv) a second sub-population of the second strain in the absence of an antifungal agent, thereby forming a fourth culture;
(c) harvesting the cells and isolating the cell walls from each culture;
(d) digesting the cell walls from each culture with a digesting agent for surface-exposed CWPs, thereby forming digested surface-exposed fungal CWPs;
(e) performing proteomic analysis on the digested surface-exposed fungal CWPs; and
(f) identifying surface-exposed epitopes of the digested surface-exposed CWPs that are:
(i) expressed in higher abundance in the first culture compared to the second culture; and/or
(ii) expressed in higher abundance in the third culture compared to the fourth culture; and/or
(iii) expressed in higher abundance in the first culture compared to the third culture.

4. The method of claim 1, comprising:
(f) identifying surface-exposed epitopes of the digested surface-exposed CWPs that are, or are determined to be, involved in cell wall remodelling pathways and/or expressed in vivo during an infection; and are expressed in higher abundance in the first culture compared to the second culture.

5. The method of claim 1, wherein the antifungal agent is capable of inducing cell wall remodelling pathways.

6. The method of claim 1, wherein the fungal species is an Aspergillus, Candida or Cryptococcus fungal species.

7. A method of generating a peptide antigen suitable for the development of antifungal antibodies, comprising:
(a) providing a surface-exposed epitope of a fungal cell wall protein (“CWP”), obtained by the method of claim 1; and
(b) generating a peptide antigen derived from the epitope, wherein the derived antigen comprises an amino acid sequence that:
(i) is comprised by the epitope, or has at least 70% sequence identity to the epitope; and
(ii) has a hydropathy index value of less than 0 for more than 50% of the amino acids comprised by the amino acid sequence; and/or
(iii) has 20% or more predicted ß-turn secondary structure.

8. A peptide antigen derived from a surface-exposed epitope of a fungal cell wall protein (“CWP”)—, obtained by the method of claim 1, suitable for the development of antifungal antibodies, wherein the derived peptide antigen:
(i) comprises an amino acid sequence that is comprised by the epitope, or that has 70% sequence identity to the epitope; and
(ii) has a hydropathy index value of less than 0 for more than 50% of the amino acids comprised by the amino acid sequence of the peptide antigen; and/or
(iii) has 20% or more predicted ß-turn secondary structure.

9. (canceled)

10. (canceled)

11. The method or peptide antigen of claim 8, wherein the CWP is Pga31, Utr2, Phr2 or Cht2.

12. (canceled)

13. An antibody, or a polynucleotide encoding the same, wherein the antibody specifically binds to a surface-exposed cell wall protein (“CWP”), wherein the CWP is Pga31, Utr2, Phr2, or Cht2.

14.-31. (canceled)

32. A method of producing an antifungal antibody that specifically bind to surface-exposed epitopes of fungal cell wall proteins (“CWPs”), comprising:
(i) providing a surface-exposed epitope of a fungal CWP identified by the method of claim 1;
(ii) screening a human antibody library against the epitope; and
(iii) producing an antibody that specifically binds to the epitope in step (ii).

33.-36. (canceled)

37. A pharmaceutical composition comprising an antibody according to claim 13, and a pharmaceutically acceptable excipient.

38.-40. (canceled)

41. A method of treatment of a fungal infection comprising administering an antibody according to claim 13, optionally wherein the fungal infection is a Candida infection, an Aspergillus infection, or a Cryptococcus infection,

42. (canceled)

43. (canceled)

44. A method for diagnosing a fungal infection in an individual which is caused by fungal species, the method comprising:
(i) contacting a biological sample obtained from the individual with an antibody according to claim 13, and
(ii) determining whether the antibody binds to the biological sample, wherein binding of the antibody to the biological sample indicates the presence of a fungal infection.

45. (canceled)

46. The method of claim 2, comprising:
(f) identifying surface-exposed epitopes of the digested surface-exposed CWPs that are, or are determined to be, involved in cell wall remodelling pathways and/or expressed in vivo during an infection; and are
(i) expressed in higher abundance in the first culture compared to the second culture; and/or
(ii) expressed in higher abundance in the third culture compared to the fourth culture; and/or
(iii) expressed in higher abundance in the first culture compared to the third culture.

47. The method of claim 5, wherein the antifungal agent is caspofungin.

48. An antibody, or a polynucleotide encoding the same, wherein the antibody specifically binds to the peptide antigen of claim 8.

49. A method of producing an antifungal antibody that specifically bind to surface-exposed epitopes of fungal cell wall proteins (“CWPs”), comprising:
(i) providing a peptide antigen of claim 8;
(ii) screening a human antibody library against the antigen; and
(iii) producing an antibody that specifically binds to the antigen in step (ii).

50. A pharmaceutical composition comprising an antibody according to claim 48, and a pharmaceutically acceptable excipient.

51. A method of treatment of a fungal infection comprising administering an antibody according to claim 48, optionally wherein the fungal infection is a Candida infection, an Aspergillus infection, or a Cryptococcus infection.

52. A method for diagnosing a fungal infection in an individual which is caused by fungal species, the method comprising:
(i) contacting a biological sample obtained from the individual with an antibody according to claim 48, and
(ii) determining whether the antibody binds to the biological sample, wherein binding of the antibody to the biological sample indicates the presence of a fungal infection.


SEQUENCE LISTING (256 sequences)

SEQ ID NO: 1 - DNA, 747 bp, Artificial Sequence
  1B11 Nucleotide Sequence
gaggtgcagctgttggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacactgtatcttcaaatgaacagcctgagagccgaggacacggccgtgtattactgtgcgagagatcgtagtgggtggggatcccttgactactggggccagggcaccctggtcaccgtctcctcactcgagggtggaggcggttcaggcggaggtggctctggcggtggcgctagcgatattgtgatgactcagtctcctgactccctggctgtgtctctgggcgagagggccaccatcaactgcaagtccagccagaatgttttatacagctccaacaataagaactacttagcttggtaccagcagaaatcaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattacagtactcctcgaacttttggccaggggaccaaggtggagatcaaa

SEQ ID NO: 2 - protein, 249 aa, Artificial Sequence
  1B11 Amino Acid Sequence
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCARDRSGWGSLDYWGQGTLVTVSSLEGGGGSGGGGSGGGASDIVMTQSPDSLAVSLGERATINCKSSQNVLYSSNNKNYLAWYQQKSGQPPKLLIYWASTRESGVPDRFSGSGSGTDFTLTISSLQAEDVAVYYCQQYYSTPRTFGQGTKVEIK

SEQ ID NO: 3 - DNA, 714 bp, Artificial Sequence
  1G4 Nucleotide Sequence
gaggtgcagctgttggagtctgggggaggcttggttcagcctggggggtccctgagagtctcctgtgcagcctctggattcacctttaacagctatgccatgagctgggtccgccaggctccagggaaggggctggagtgggtctcaactgttagtggaagtggtggtagcacatactacgcagactccgtgaagggccggttcaccatctccagagacaattccaagaacacgctgtatctgcaaatgaacagcctgagagctgaggacacggctgtgtattactgtgcgagagggtacttcgatctctggggccgtggaaccctggtcaccgtctcctcactcgagggtggaggcggttcaggcggaggtggctctggcggtggcgctagcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagacttacaccaccccgctcactttcggcggagggaccaaggtggaaatcaaa

SEQ ID NO: 4 - protein, 238 aa, Artificial Sequence
  1G4 Amino Acid Sequence
EVQLLESGGGLVQPGGSLRVSCAASGFTFNSYAMSWVRQAPGKGLEWVSTVSGSGGSTYYADSVKGRFTISRDNSKNTLYLQMNSLRAEDTAVYYCARGYFDLWGRGTLVTVSSLEGGGGSGGGGSGGGASDIQMTQSPSSLSASVGDRVTITCRASQSISSYLNWYQQKPGKAPKLLIYAASSLQSGVPSRFSGSGSGTDFTLTISSLQPEDFATYYCQQTYTTPLTFGGGTKVEIK

SEQ ID NO: 5 - DNA, 744 bp, Artificial Sequence
  1A2 Nucleotide Sequence
caggttcagcttgtgcagtctggggctgaggtgaacaagcctggggcctcagtgaaggtttcctgcaaggcttctggatacaccttcactagctatgctatgcattgggtgcgccaggcccccggacaaaggcttgagtggatgggatggatcaacgctggcaatggtaacacaaaatattcacagaagttccagggcagagtcaccattaccagggacacatccgcgagcacagcctacatggagctgagcagcctgagatctgaagacacggctgtgtattactgtgcgaggggcggagcagcagctggttactacatggacgtctggggcaaaggaaccctggtcaccgtctcctcactcgagggtggaggcggttcaggcggaggtggctctggcggtggcgctagcaattttatgctgactcagccccactctgtgtcggagtctccggggaagacggtaaccatctcctgcacccgcagcagtggcagcattgccagcaactatgtgcagtggtaccagcagcgcccgggcagttcccccaccactgtgatctatgacgataaccaaagaccctctggggtccctgatcggttctctgcctccatcgacagctcctccaactctgcctccctcaccatctctggactgaagactgaggacgaggctgactactactgtcagtcttatgatagcagcatcgtggtattcggcggagggaccaagctgaccgtcctaggt

SEQ ID NO: 6 - protein, 248 aa, Artificial Sequence
  1A2 Amino Acid Sequence
QVQLVQSGAEVNKPGASVKVSCKASGYTFTSYAMHWVRQAPGQRLEWMGWINAGNGNTKYSQKFQGRVTITRDTSASTAYMELSSLRSEDTAVYYCARGGAAAGYYMDVWGKGTLVTVSSLEGGGGSGGGGSGGGASNFMLTQPHSVSESPGKTVTISCTRSSGSIASNYVQWYQQRPGSSPTTVIYDDNQRPSGVPDRFSASIDSSSNSASLTISGLKTEDEADYYCQSYDSSIVVFGGGTKLTVLG

SEQ ID NO: 7 - DNA, 729 bp, Artificial Sequence
  A7 Nucleotide Sequence
caggtgcagctggtggagtctgggggaggcgtggtccagcctgggaggtccctgagactctcctgtgcagcctctggattcaccttcagtagctatggcatgcactgggtccggcaagctccagggaagggcctggagtgggtctcaggtattagttggaatagtggtagcataggctatgcggactccgtgaagggccgattcaccatctccagagacaattccaagaacacgctgtatctgcaaatgaacagcctgagagctgaggacacggctgtgtattactgtgcgactaagtacggtatggacgtctggggccaagggacaatggtcaccgtctcttcactcgagggtggaggcggttcaggcggaggtggctctggcggtggcgctagccagtctgtgctgactcagccaccctcggtgtccaagaccttgagacagaccgccacactcacctgcactgggagcagcagcaatgttgccaaccaaggagcaacttggctgcagcagcaccagggccaccctcccaaactcctatcttacaggaataacaaccggccctcagggatctcagagagattctctgcatccaggtcaggaagcactgcctccctgaccattactggactccagcctgacgacgaggctgactattattgctcagcatgggacagcagcctcagtgcttgggtgttcggcggagggaccaagctgaccgtcctaggt

SEQ ID NO: 8 - protein, 243 aa, Artificial Sequence
  A7 Amino Acid Sequence
QVQLVESGGGVVQPGRSLRLSCAASGFTFSSYGMHWVRQAPGKGLEWVSGISWNSGSIGYADSVKGRFTISRDNSKNTLYLQMNSLRAEDTAVYYCATKYGMDVWGQGTMVTVSSLEGGGGSGGGGSGGGASQSVLTQPPSVSKTLRQTATLTCTGSSSNVANQGATWLQQHQGHPPKLLSYRNNNRPSGISERFSASRSGSTASLTITGLQPDDEADYYCSAWDSSLSAWVFGGGTKLTVLG

SEQ ID NO: 9 - DNA, 768 bp, Artificial Sequence
  1B1 Nucleotide Sequence
cagggtcagctggtgcagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgagaatagcccatcccaggaccagggggggaaaggaagttgaatacttccagcactggggccagggaaccctggtcaccgtctcctcactcgagggtggaggcggttcaggcggaggtggctctggcggtggcgctagcgatattgtgatgacccagactccactctcctcacctgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgttggggtttattactgcatgcaaggtacacactggcctccctcgttcggccaagggaccaagctggagatcaaacgt

SEQ ID NO: 10 - protein, 256 aa, Artificial Sequence
  1B1 Amino Acid Sequence
QGQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARIAHPRTRGGKEVEYFQHWGQGTLVTVSSLEGGGGSGGGGSGGGASDIVMTQTPLSSPVTLGQPASISCRSSQSLVYSDGNTYLNWFQQRPGQSPRRLIYKVSNRDSGVPDRFSGSGSGTDFTLKISRVEAEDVGVYYCMQGTHWPPSFGQGTKLEIKR

SEQ ID NO: 11 - DNA, 744 bp, Artificial Sequence
  B5 Nucleotide Sequence
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgaaaaccgaagacacggcccagtattactgtgttagagttcggaggtcgggtatggctcggggacttattgactactggggccagggcaccctggtcaccgtctcctcactcgagggtggaggcggttcaggcggaggtggctctggcggtggcgctagctcctatgagctgactcaggaccctgctgtgtctgtggccttgggacagacagtcaggatcacatgccaaggagacatcctcagaagctattatgcaagttggtaccagcagaagccaggacaggcccctgtacttgtcatctatggtaaaaacaaccggccctcagggatcccagaccgattctctggctccagctcaggaaacacagcttccttgaccatcactggggctcaggcggaagatgaggctgactattactgtaactcccgggacagcagtggtaaccataaggtgttcggcggagggaccaaggtcaccgtcctaggt

SEQ ID NO: 12 - protein, 248 aa, Artificial Sequence
  B5 Amino Acid Sequence
QVQLVESGGGVVQPGRSLRLSCAASGFTFSSYGMHWVRQAPGKGLEWVAVISYDGSNKYYADSVKGRFTISRDNSKNTLYLQMNSLKTEDTAQYYCVRVRRSGMARGLIDYWGQGTLVTVSSLEGGGGSGGGGSGGGASSYELTQDPAVSVALGQTVRITCQGDILRSYYASWYQQKPGQAPVLVIYGKNNRPSGIPDRFSGSSSGNTASLTITGAQAEDEADYYCNSRDSSGNHKVFGGGTKVTVLG

SEQ ID NO: 13 - DNA, 744 bp, Artificial Sequence
  1C2 Nucleotide Sequence
  misc_feature (309)..(309): n is a, c, g, or t
caggtccagcttgtgcagtctggggctgaggtgaagaagcctggggcctcagtgaaggtttcctgcaaggcttctggatacaccttcactagctatgctatgcattgggtgcgccaggcccccggacaaaggcttgagtggatgggatggatcaacgctggcaatggtaacacaaaatattcacagaagttccagggcagagtcaccattaccagggacacatccgcgagcacagcctacatggagctgagcagcctgagatctgaagacacggctgtgtattactgtgcgaggggcggagcagcagcnggttactacatggacgtctggggcaaaggaaccctggtcaccgtctcctcactcgagggtggaggcggttcaggcggaggtggctctggcggtggcgctagcaattttatgctgactcagccccactctgtgtcggagtctccggggaagacggtaaccatctcctgcacccgcagcagtggcagcattgccagcaactatgtgcagtggtaccagcagcgcccgggcagttcccccaccactgtgatctatgacgataaccaaagaccctctggggtccctgatcggttctctgcctccatcgacagctcctccaactctgcctccctcaccatctctggactgaagactgaggacgaggctgactactactgtcagtcttatgatagcagcatcgtggtattcggcggagggaccaagctgaccgtcctaggt

SEQ ID NO: 14 - protein, 248 aa, Artificial Sequence
  1C2 Amino Acid Sequence
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARGGAAAGYYMDVWGKGTLVTVSSLEGGGGSGGGGSGGGASNFMLTQPHSVSESPGKTVTISCTRSSGSIASNYVQWYQQRPGSSPTTVIYDDNQRPSGVPDRFSASIDSSSNSASLTISGLKTEDEADYYCQSYDSSIVVFGGGTKLTVLG

SEQ ID NO: 15 - DNA, 756 bp, Artificial Sequence
  C5 Nucleotide Sequence
gaggtgcagctggtggagtctgggggaggcgtggtccagcctgggaggtccctgaggctctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccacctccagagacaattccaagaacacgctgtatctgcaaatgaacagcctgagagctgaggacacggctgtgtattactgtgcccggatagcagtggctggtcgatcccaaaatgttgactactggggccagggcaccctggtcaccgtctcctcactcgagggtggaggcggttcaggcggaggtggctctggcggtggcgctagcgatattgtgatgacacagactccactcccctcacctgtcacccttggacagccggcctccatctcctgcaggtctagtcaaagcctcctacacagtgatggaagcacctacttgaattggtttcaccagaggccaggccaatctccaaggcgcctaatttataaggtttctaggcgggactctggggtcccagacagattcagcggcagtgggtcaggcactgatttcacactgaacatcagcagggtggaggctgaggatgttggggtttattactgcatgcaaggtacacgctggcccccaatcaccttcggacaagggacacgactggagattaaacgt

SEQ ID NO: 16 - protein, 252 aa, Artificial Sequence
  C5 Amino Acid Sequence
EVQLVESGGGVVQPGRSLRLSCAASGFTFSSYAMHWVRQAPGKGLEWVAVISYDGSNKYYADSVKGRFTTSRDNSKNTLYLQMNSLRAEDTAVYYCARIAVAGRSQNVDYWGQGTLVTVSSLEGGGGSGGGGSGGGASDIVMTQTPLPSPVTLGQPASISCRSSQSLLHSDGSTYLNWFHQRPGQSPRRLIYKVSRRDSGVPDRFSGSGSGTDFTLNISRVEAEDVGVYYCMQGTRWPPITFGQGTRLEIKR

SEQ ID NO: 17 - DNA, 738 bp, Artificial Sequence
  C8 Nucleotide Sequence
gaggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaggaaccaagggtaaggactactactacatggacgtctggggcaaaggcaccctggtcaccgtctcctcactcgagggtggaggcggttcaggcggaggtggctctggcggtggcgctagcgacatccagatgacccagtctccatcttccctgtctgcatctgtaggagacagagtcaccatcacttgccgggcaagtcagagcattagcacctatttaaattggtatcagcagaagccagggacagcccctaacctcctgatctatggtgcatccaatttgcaaagtggggtcccatcaaggttcagtggcagtggatctgggacagatttcactctcaccatcagcagtctgcaacctgaagattttgcaacttactactgtcaacagagttacagtacccctctcactttcggcggagggaccaaagtggatatcaaacgt

SEQ ID NO: 18 - protein, 246 aa, Artificial Sequence
  C8 Amino Acid Sequence
EVQLVQSGAEVKKPGSSVKVSCKASGGTFSSYAISWVRQAPGQGLEWMGIINPSGGSTSYAQKFQGRVTMTRDTSTSTVYMELSSLRSEDTAVYYCARGTKGKDYYYMDVWGKGTLVTVSSLEGGGGSGGGGSGGGASDIQMTQSPSSLSASVGDRVTITCRASQSISTYLNWYQQKPGTAPNLLIYGASNLQSGVPSRFSGSGSGTDFTLTISSLQPEDFATYYCQQSYSTPLTFGGGTKVDIKR

SEQ ID NO: 19 - DNA, 723 bp, Artificial Sequence
  C9 Nucleotide Sequence
caggtgcagctggtggagtctgggggaggcgtggtccagcctgggaggtccctgagactctcctgtgcagcctctggattctccttcagtagttatggtgtgcactgggtccgccaggctccaggcaaggggctggagtgggtggcaggtatatcatatgatggaaacaataaatactacacagactccgtgaagggccgattcaccatctccagagacaattccaagaacacgctgtatctgcaaatgaacagcctgagagctgaggacacggctgtgtattactgtgcgaaagggaggctatggattgactactggggccagggaaccctggtcaccgtctcctcactcgagggtggaggcggttcaggcggaggtggctctggcggtggcgctagcgatattgtgatgacgcagtctccagccaccctgtctctgtccccaggggaaagagccaccctctcctgcagggccagtcagagtattgccagaaacttagcctggtaccagctcagacctggccaggctcccaggctcctcatctatggtgcatcaaccagggccactggtgtcccagacagattcagcggcagtgggtctgggacagatttcactctcaccatcagcagcctgcaggcagaagatgtggcagtttatttctgtcagcaatatgaaactcttccgtacacttttggccgggggaccaaagtggatatcaaacgt

SEQ ID NO: 20 - protein, 241 aa, Artificial Sequence
  C9 Amino Acid Sequence
QVQLVESGGGVVQPGRSLRLSCAASGFSFSSYGVHWVRQAPGKGLEWVAGISYDGNNKYYTDSVKGRFTISRDNSKNTLYLQMNSLRAEDTAVYYCAKGRLWIDYWGQGTLVTVSSLEGGGGSGGGGSGGGASDIVMTQSPATLSLSPGERATLSCRASQSIARNLAWYQLRPGQAPRLLIYGASTRATGVPDRFSGSGSGTDFTLTISSLQAEDVAVYFCQQYETLPYTFGRGTKVDIKR

SEQ ID NO: 21 - DNA, 732 bp, Artificial Sequence
  C10 Nucleotide Sequence
caggtccagcttgtgcagtctggggctgaggtgaagaagcctggggcctcagtgaaggtttcctgcaaggcttctggatacaccttcactagctatgctatgcattgggtgcgccaggcccccggacagaggcttgagtggatgggatggatcaacgctggcaatgataacacaaaatattcacagaagttccagggcagagtcaccattaccagggacacatccgcgggcacagcctacatggagatgagcggcctgacatctgaggacacggctgtgtattactgtgcgagaggcacctactacatagacgtctggggcaaaggaaccctggtcaccgtctcctcactcgagggtggaggcggttcaggcggaggtggctctggcggtggcgctagcaattttatgctgactcagccccactctgtgtcggagtctgcggggaagacggtaaccatctcctgcacccgcagcagtggcagcattgccagcaactatgtgcagtggtaccagcagcgcccgggcagttcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcagcaatcaggtgttcggcggagggaccaagctgaccgtcctaggt

SEQ ID NO: 22 - protein, 244 aa, Artificial Sequence
  C10 Amino Acid Sequence
QVQLVQSGAEVKKPGASVKVSCKASGYTFTSYAMHWVRQAPGQRLEWMGWINAGNDNTKYSQKFQGRVTITRDTSAGTAYMEMSGLTSEDTAVYYCARGTYYIDVWGKGTLVTVSSLEGGGGSGGGGSGGGASNFMLTQPHSVSESAGKTVTISCTRSSGSIASNYVQWYQQRPGSSPTTVIYEDNQRPSGVPDRFSGSIDSSSNSASLTISGLKTEDEADYYCQSYDSSNQVFGGGTKLTVLG

SEQ ID NO: 23 - DNA, 729 bp, Artificial Sequence
  1D2 Nucleotide Sequence
caggttcagctggtgcagtctggggctgaggtgaagaagcctggggcctcagtgaaggtctcctgcaaggcttctggatacagcttcacgaattatgacatctactgggtgcgacaggccactggacaagggcttgagtgggtgggattcataaatccgaagactggtaaaacaggctatgcacagaagttccagggcagagtcaccatgagcagggacacttccataaccacagcctacatggaactgaacagcctgacatctgaagacacggccgtgtattactgtgcgagcatttccggatactggggccagggcaccctggtcaccgtctcctcactcgagggtggaggcggttcaggcggaggtggctctggcggtggcgctagcgatattgtgatgacccagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcactgatttcacactgaaaatcagcagggtggaggctgaggatgctgggctttattactgcatgcaaggttcacactggcctccgacttttggccaggggaccaaggtggagatcaaacgt

SEQ ID NO: 24 - protein, 243 aa, Artificial Sequence
  1D2 Amino Acid Sequence
QVQLVQSGAEVKKPGASVKVSCKASGYSFTNYDIYWVRQATGQGLEWVGFINPKTGKTGYAQKFQGRVTMSRDTSITTAYMELNSLTSEDTAVYYCASISGYWGQGTLVTVSSLEGGGGSGGGGSGGGASDIVMTQSPLSLPVTLGQPASISCRSSQSLVYSDGNTYLNWFQQRPGQSPRRLIYKVSNRDSGVPDRFSGSGSGTDFTLKISRVEAEDAGLYYCMQGSHWPPTFGQGTKVEIKR

SEQ ID NO: 25 - DNA, 747 bp, Artificial Sequence
  D3 Nucleotide Sequence
caaatgcagctggtgcagtctggggctgaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacttaagacaggcgaccagctgcccgatatctggggccaagggacaatggtcaccgtctcttcactcgagggtggaggcggttcaggcggaggtggctctggcggtggcgctagcgatattgtgatgacgcagactccactctctctgtccgtcacccctggacagccggcctccatctcctgcaagtctagtcagagcctcctgcatagtgatggaaagacctatttgtattggtacctgcagaagccaggccagcctccacagctcctgatctatgaagtttccaaccggttctctggagtgccagataggttcagtggcagcgggtcagggacagacttcacactgaaaatcagccgggtggaggctgaggatgttggggtttattactgcatgcaaactatacagcttcctgccacgttcggcggagggaccaagctggagatcagacgt

SEQ ID NO: 26 - protein, 249 aa, Artificial Sequence
  D3 Amino Acid Sequence
QMQLVQSGAEVKKPGESLKISCKGSGYSFTSYWIGWVRQMPGKGLEWMGIIYPGDSDTRYSPSFQGQVTISADKSISTAYLQWSSLKASDTAMYYCARLKTGDQLPDIWGQGTMVTVSSLEGGGGSGGGGSGGGASDIVMTQTPLSLSVTPGQPASISCKSSQSLLHSDGKTYLYWYLQKPGQPPQLLIYEVSNRFSGVPDRFSGSGSGTDFTLKISRVEAEDVGVYYCMQTIQLPATFGGGTKLEIRR

SEQ ID NO: 27 - DNA, 750 bp, Artificial Sequence
  1F4 Nucleotide Sequence
gaggtgcagctgttggagtcggggggaggcttggtccagcctgggggatccctgagactctcttgtgcagcctctggattcaccttcagcaattatggcataaactgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgagtccgattcggggagttaagcagcactggggccagggcaccctggtcaccgtctcctcactcgagggtggaggcggttcaggcggaggtggctctggcggtggcgctagccagtctgtgctgactcagccgtcttccctctctgcatctcctggagcatcagccagtctcacctgcaccttgcgcagtggcatcaatgttggtacctacaggatatactggtatcagcagaagccagggggtcctccccagtatctcctgacgtccatatcaggctcaaattaccagcagggctccggagtccccaggcgcttctctgcatacaaagatgcttcggccaatgcagggattttactcatctctgggctccagtctgaggatgaggctgactattattgtatgatttggcacagcagcgctgtggtcttcggcggagggaccaagctgaccgtcctaggt

SEQ ID NO: 28 - protein, 250 aa, Artificial Sequence
  1F4 Amino Acid Sequence
EVQLLESGGGLVQPGGSLRLSCAASGFTFSNYGINWVRQAPGKGLEWVSAISGSGGSTYYADSVKGRFTISRDNSKNTLYLQMNSLRAEDTAVYYCASPIRGVKQHWGQGTLVTVSSLEGGGGSGGGGSGGGASQSVLTQPSSLSASPGASASLTCTLRSGINVGTYRIYWYQQKPGGPPQYLLTSISGSNYQQGSGVPRRFSAYKDASANAGILLISGLQSEDEADYYCMIWHSSAVVFGGGTKLTVLG

SEQ ID NO: 29 - DNA, 729 bp, Artificial Sequence
  1H3 Nucleotide Sequence
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaacttagtagggaaaatgcttttgatatctggggccaagggacaatggtcaccgtctcttcactcgagggtggaggcggttcaggcggaggtggctctggcggtggcgctagcgacatccagatgacccagtctccatcctccctgtctgcatctgtaggagacagagtcaccatcacttgccaggcgagtcaggacatttggaaatatgtaaattggtatcagcagaaaccagagaaggcccctaagtccctgatctatgctgcatccagtttgcaaagtggggtcccatcaaggttcagcggcagtggatctgggacagaattcactctcacaatcagcagcctgcagcctgaagattttgcaacttactactgtcaacagagttacagtatgcctcggacgttcggccaagggaccaagctggagatcaaacgt

SEQ ID NO: 30 - protein, 243 aa, Artificial Sequence
  1H3 Amino Acid Sequence
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKLSRENAFDIWGQGTMVTVSSLEGGGGSGGGGSGGGASDIQMTQSPSSLSASVGDRVTITCQASQDIWKYVNWYQQKPEKAPKSLIYAASSLQSGVPSRFSGSGSGTEFTLTISSLQPEDFATYYCQQSYSMPRTFGQGTKLEIKR

SEQ ID NO: 31 - DNA, 720 bp, Artificial Sequence
  3C5 Nucleotide Sequence
gaggtgcagctgttggagtctgggggaggcgtggtccagcctgggaggtccctgagactctcctgtgcagcgtctggattcaccttcagtagctatggcatgcactgggtccgccaggctccaggcaaggggctggagtgggtggcagttatatcatatgctggaagcaataaatactacgcagactccgtgaagggccgattcaccatctccagagacaattccaagaacacgctgtatctgcaaatgaacagcctgagagctgaggacacggctgtgtattactgtgcgaaaggtcagagaatggacgtctggggcaaagggacaatggtcaccgtctcttcactcgagggtggaggcggttcaggcggaggtggctctggcggtggcgctagcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctatcactttcggccctgggaccaaagtggatatcaaacgt

SEQ ID NO: 32 - protein, 240 aa, Artificial Sequence
  3C5 Amino Acid Sequence
EVQLLESGGGVVQPGRSLRLSCAASGFTFSSYGMHWVRQAPGKGLEWVAVISYAGSNKYYADSVKGRFTISRDNSKNTLYLQMNSLRAEDTAVYYCAKGQRMDVWGKGTMVTVSSLEGGGGSGGGGSGGGASDIQMTQSPSSLSASVGDRVTITCRASQSISSYLNWYQQKPGKAPKLLIYAASSLQSGVPSRFSGSGSGTDFTLTISSLQPEDFATYYCQQSYSTPITFGPGTKVDIKR

SEQ ID NO: 33 - DNA, 744 bp, Artificial Sequence
  2C7 Nucleotide Sequence
gaagtgcagctggtgcagtctggggctgaggtgaagaagcctggggcctcagtgaaggtctcctgcaaggcttctggatacaccttcaccggctactatatgcactgggtgcgacaggcccctggacaagggcttgagtggatgggacggatcaaccctaacagtggtgacacaaactatgcacagaagtttcagggcagggtcaccatgaccagggacacgtccatcagcacagcctacatggagctgaacaggctgagatctgacgacacggccgtgtattactgtgcgagagatcgcgaccgaggtatggacgtctggggccaagggacaatggtcaccgtctcttcactcgagggtggaggcggttcaggcggaggtggctctggcggtggcgctagcgacatccagatgacccagtctccagactcactgactttgtctctgggcgagagggccaccgtcaactgcgtgtccagccaaaatcttttattcaactccaacaaaaagaactgcttagcttggtatcagcaaaaagcaggacagcctcctaggctggtcatttactgggcatccacccggaaatccggggtccctgaccgattcagtggcagcgggtctgggacagatttcactctcaccatcagcagtctacaacctgaagattttgcaacttactactgtcaacggagttacagttccccgttcacttttggccaggggaccaaggtggagatcaaacgt

SEQ ID NO: 34 - protein, 248 aa, Artificial Sequence
  2C7 Amino Acid Sequence
EVQLVQSGAEVKKPGASVKVSCKASGYTFTGYYMHWVRQAPGQGLEWMGRINPNSGDTNYAQKFQGRVTMTRDTSISTAYMELNRLRSDDTAVYYCARDRDRGMDVWGQGTMVTVSSLEGGGGSGGGGSGGGASDIQMTQSPDSLTLSLGERATVNCVSSQNLLFNSNKKNCLAWYQQKAGQPPRLVIYWASTRKSGVPDRFSGSGSGTDFTLTISSLQPEDFATYYCQRSYSSPFTFGQGTKVEIKR

SEQ ID NO: 35 - DNA, 768 bp, Artificial Sequence
  1F5 Nucleotide Sequence
caggtgcagctggtgcagtctggggctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagattcgggatattttgactggttcccctactactactactacggtatggacgtctggggccaagggacaatggtcaccgtctcttcactcgagggtggaggcggttcaggcggaggtggctctggcggtggcgctagcaattttatgctgactcagccccactctgtgtcggagtctccggggaagacggtaaccatctcctgcacccgcagcagtggcagcattgccagcaactatgtgcagtggtaccagcagcgcccgggcagtgcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcgtcaatcggggaatattcggcggagggacccagctcaccgttttaggt

SEQ ID NO: 36 - protein, 256 aa, Artificial Sequence
  1F5 Amino Acid Sequence
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDSGYFDWFPYYYYYGMDVWGQGTMVTVSSLEGGGGSGGGGSGGGASNFMLTQPHSVSESPGKTVTISCTRSSGSIASNYVQWYQQRPGSAPTTVIYEDNQRPSGVPDRFSGSIDSSSNSASLTISGLKTEDEADYYCQSYDSVNRGIFGGGTQLTVLG

SEQ ID NO: 37 - DNA, 753 bp, Artificial Sequence
  1G10 Nucleotide Sequence
caggtgcagctggtggagtctggtcctacgctggtgaaacccacacagaccctcacgctgacctgcaccttctctgggttctcactcagcactagtggagtgggtgtgggctggatccgtcagcccccaggaaaggccctggagtggcttgcactcatttatcggaatgaagataagcgctacagcccatctctggagcgcaggctcaccatcaccaaggacacctccaaaaaccaggtggcccttacaatgaccgacatggcccctgaggacacagccacatattactgtgcacacagggcggctacagcagctgtcctagactactggggccagggaaccctggtcaccgtctcctcactcgagggtggaggcggttcaggcggaggtggctctggcggtggcgctagcgatattgtgatgactcagtctccagactccctggctgtgtctctgggcgagagggccaccatgaactgcaagtccagccatagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcagcctgaagatgtggcagtttatttctgtcagcagtattatagtatcccattcactttcggcccggggaccaagctggagatcaaacgt

SEQ ID NO: 38 - protein, 251 aa, Artificial Sequence
  1G10 Amino Acid Sequence
QVQLVESGPTLVKPTQTLTLTCTFSGFSLSTSGVGVGWIRQPPGKALEWLALIYRNEDKRYSPSLERRLTITKDTSKNQVALTMTDMAPEDTATYYCAHRAATAAVLDYWGQGTLVTVSSLEGGGGSGGGGSGGGASDIVMTQSPDSLAVSLGERATMNCKSSHSVLYSSNNKNYLAWYQQKPGQPPKLLIYWASTRESGVPDRFSGSGSGTDFTLTISSLQPEDVAVYFCQQYYSIPFTFGPGTKLEIKR

SEQ ID NO: 39 - DNA, 759 bp, Artificial Sequence
  1H7 Nucleotide Sequence
gaggtgcagctgttggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaagctattagggtatagcagtggctggtaccgtccgggggcttttgatatctggggccaagggacaatggtcaccgtctcttcactcgagggtggaggcggttcaggcggaggtggctctggcggtggcgctagccagtctgcgctgactcagcctgcctccgtgtctgggtctcctggacagtcgatcaccatctcctgcactggaaccagcagtgacgttggtagatataactatgtctcctggtaccaacaacacccaggcaaagcccccaaactcatgatttatgatgtcagtaatcggccctcaggtgtttctaatcgcttctctggctccaagtctggcaacacggcctccctgaccatctctgggctccaggctgaggacgaggctgattattactgcagctcatatacaagcagcagcactcgggtgttcggcggagggaccaaggtcaccgtcctaggt

SEQ ID NO: 40 - protein, 253 aa, Artificial Sequence
  1H7 Amino Acid Sequence
EVQLLESGGGVVQPGRSLRLSCAASGFTFSSYGMHWVRQAPGKGLEWVAVISYDGSNKYYADSVKGRFTISRDNSKNTLYLQMNSLRAEDTAVYYCAKLLGYSSGWYRPGAFDIWGQGTMVTVSSLEGGGGSGGGGSGGGASQSALTQPASVSGSPGQSITISCTGTSSDVGRYNYVSWYQQHPGKAPKLMIYDVSNRPSGVSNRFSGSKSGNTASLTISGLQAEDEADYYCSSYTSSSTRVFGGGTKVTVLG

SEQ ID NO: 41 - DNA, 753 bp, Artificial Sequence
  2B1 Nucleotide Sequence
caggtgcagctggtggagtctggggctgaggtgaagaagcctggggcctcagtgaaggtctcctgcaaggcttctggatacaccttcaccggctactatatgcactgggtgcgacaggcccctggacaagggcttgagtggatgggacggatcaaccctaacagtggtggcacaaactatgcacagaagtttcagggcagggtcaccatgaccagggacacgtccatcagcacagcctacatggagctgagcaggctgagatctgacgacacggccgtgtattactgtgcgagagtggattacgatattttgactggttattatcccccccaggacatggacgtctggggcaaagggacaatggtcaccgtctcttcactcgagggtggaggcggttcaggcggaggtggctctggcggtggcgctagcgaaacgacactcacgcagtctccagcattcatgtcagcgactccaggagacaaagtcaacatctcctgcaaagccagccaagacattgatgatgatatgaactggtaccaacagaaaccaggagaagctcctattttcattattcaagaagctactactctcgttcctggaatcccacctcgattcagtggcagcgggtatggaacagattttaccctcacaattaataacatagaatctgacgatgctgcatattacttctgtctacaacatgataatttcccgtacacttttggccaggggaccaaggtggaaatcaaacgt

SEQ ID NO: 42 - protein, 251 aa, Artificial Sequence
  2B1 Amino Acid Sequence
QVQLVESGAEVKKPGASVKVSCKASGYTFTGYYMHWVRQAPGQGLEWMGRINPNSGGTNYAQKFQGRVTMTRDTSISTAYMELSRLRSDDTAVYYCARVDYDILTGYYPPQDMDVWGKGTMVTVSSLEGGGGSGGGGSGGGASETTLTQSPAFMSATPGDKVNISCKASQDIDDDMNWYQQKPGEAPIFIIQEATTLVPGIPPRFSGSGYGTDFTLTINNIESDDAAYYFCLQHDNFPYTFGQGTKVEIKR

SEQ ID NO: 43 - DNA, 741 bp, Artificial Sequence
  2C8 Nucleotide Sequence
caagtgcagctggttgaatctgggggaagcgtggtccacccggggaggtccctgagactctcctgtgcggcctctggattcaccttccgtagctatgctatgcactgggtccgccaggctccaggcaaggggctggagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgagattagcgactacggtgactacgctgaatgcttttgatatctggggccaaggcaccctggtcaccgtctcctcactcgagggtggaggcggttcaggcggaggtggctctggcggtggcgctagcgacatccagatgacccagtctccttccaccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctggggcagatttcactctcaccatcagcagcctgcagcctgaagattttgcaacttattactgtcaacagtttaatagttaccctcgaacgttcggccaagggaccaagctggagatcaaacgt

SEQ ID NO: 44 - protein, 247 aa, Artificial Sequence
  2C8 Amino Acid Sequence
QVQLVESGGSVVHPGRSLRLSCAASGFTFRSYAMHWVRQAPGKGLEWVAVISYDGSNKYYADSVKGRFTISRDNSKNTLYLQMNSLRAEDTAVYYCARLATTVTTLNAFDIWGQGTLVTVSSLEGGGGSGGGGSGGGASDIQMTQSPSTLSASVGDRVTITCRASQSISSYLNWYQQKPGKAPKLLIYAASSLQSGVPSRFSGSGSGADFTLTISSLQPEDFATYYCQQFNSYPRTFGQGTKLEIKR

SEQ ID NO: 45 - DNA, 762 bp, Artificial Sequence
  2D3 Nucleotide Sequence
  misc_feature (759)..(759): n is a, c, g, or t
caggtccagcttgtgcagtctggggctgaggtgaagaagcctggggcctcagtgaaggtttcctgcaaggcttctggatacaccttcactagctatgctatgcattgggtgcgccaggcccccggacaaaggcttgagtggatgggatggatcaacgctggcaatggtaacacaaaatattcacagaagttccagggcagagtcaccattaccagggacacatccgcgagcacagcctacatggagctgagcagcctgagatctgaagacacggctgtgtattactgtgcgaggggcagaatagcagctcgtcggggggactactactactacggtatggacgtctggggccaaggaaccctggtcaccgtctcctcactcgagggtggaggcggttcaggcggaggtggctctggcggtggcgctagcaattttatgctgactcagccgcactctgtgtcggagtctccggggaagacggtaatcatctcctgcacccgcagcagtggcaacattgccagcaactatgtgcagtggtaccggcagcgcccgggcagtgcccccacctctgtgatctatgaggataaccaaagaccctctggggtccctgatcggttctctggctccatcgacagctcctccaactctgcctccctcaccatctctggactgcagactgaggacgaggctgactactactgtcagtcttatgatagcagcacccgggtgttcggcggagggaccaaggtcaccgtcctnggt

SEQ ID NO: 46 - protein, 254 aa, Artificial Sequence
  2D3 Amino Acid Sequence
QVQLVQSGAEVKKPGASVKVSCKASGYTFTSYAMHWVRQAPGQRLEWMGWINAGNGNTKYSQKFQGRVTITRDTSASTAYMELSSLRSEDTAVYYCARGRIAARRGDYYYYGMDVWGQGTLVTVSSLEGGGGSGGGGSGGGASNFMLTQPHSVSESPGKTVIISCTRSSGNIASNYVQWYRQRPGSAPTSVIYEDNQRPSGVPDRFSGSIDSSSNSASLTISGLQTEDEADYYCQSYDSSTRVFGGGTKVTVLG

SEQ ID NO: 47 - DNA, 771 bp, Artificial Sequence
  2F1 Nucleotide Sequence
  misc_feature (746)..(746): n is a, c, g, or t
caagttcagctgttgcagtctggagctgaggtgaagaagcctggggcctcagtgaaggtctcctgcaaggcttctggttacacctttaccagctacggtatcagctgggtgcgacaggcccctggacaagggcttgagtggatgggatggatcagcgcttacaatggtaacacaaactatgcacagaagctccagggcagagtcacgattaccgcggacaaatccacgagcacagcctacatggagctgagcagcctgagatctgaggacacggccgtgtattactgtgcgagagagggtcgggatattgtagtggtggtagctgctacatgggactactactactacggtatggacgtctggggccaagggacaatggtcaccgtctcttcactcgagggtggaggcggttcaggcggaggtggctctggcggtggcgctagcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctacgatgcatccaatttggacacaggggtcccatcaaggttcagtggaagtggatctgggacagacttcactctcaccatcagcagtctgcaccctgaagattttgcaacttactactgtcaagagactcacagtgtccctccttgcaattttggccnggggaccaaggtggagatcaaacgt

SEQ ID NO: 48 - protein, 257 aa, Artificial Sequence
  2F1 Amino Acid Sequence
  misc_feature (249)..(249): Xaa can be any naturally occurring amino acid
QVQLLQSGAEVKKPGASVKVSCKASGYTFTSYGISWVRQAPGQGLEWMGWISAYNGNTNYAQKLQGRVTITADKSTSTAYMELSSLRSEDTAVYYCAREGRDIVVVVAATWDYYYYGMDVWGQGTMVTVSSLEGGGGSGGGGSGGGASDIQMTQSPSSLSASVGDRVTITCRASQSISSYLNWYQQKPGKAPKLLIYDASNLDTGVPSRFSGSGSGTDFTLTISSLHPEDFATYYCQETHSVPPCNFGXGTKVEIKR

SEQ ID NO: 49 - DNA, 759 bp, Artificial Sequence
  2F11 Nucleotide Sequence
caggtgcagctggtggagtctgggggaggcgtggtccagcctgggaggtccctgagactctcctgtgcagcctctggattcaccttcagtagctatgctatgcactgggtccgccaggctccaggcaaggggctagagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgagaatgtataactggaaccagcgcggcgggatacatgatgcttttgatatctggggccaagggacaatggtcaccgtctcttcactcgagggtggaggcggttcaggcggaggtggctctggcggtggcgctagctcctatgagctgactcaggaccctgctgtgtctgtggccttgggacagacagtcaggatcacatgccaaggagacagcctcagaacctattatgcaagctggtaccagcagaagccaggacaggcccctaaacttgtcatctatggtaaaaacaaccggccctcacggatcccagaccgattctctggctccacctcaggaaacacagcttctttgaccatcacaggggctcaggcggaagatgaggctgactattactgtaactcccgggacagcagtgttaaccgtcgtgatgtggtcttcggcggagggaccaaggtcaccgtcctaggt

SEQ ID NO: 50 - protein, 253 aa, Artificial Sequence
  2F11 Amino Acid Sequence
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARMYNWNQRGGIHDAFDIWGQGTMVTVSSLEGGGGSGGGGSGGGASSYELTQDPAVSVALGQTVRITCQGDSLRTYYASWYQQKPGQAPKLVIYGKNNRPSRIPDRFSGSTSGNTASLTITGAQAEDEADYYCNSRDSSVNRRDVVFGGGTKVTVLG

SEQ ID NO: 51 - DNA, 735 bp, Artificial Sequence
  2H6 Nucleotide Sequence
gaagtgcagctggtgcagtctggggctgaggtgaagaagcctggggcctcagtgaaggtctcctgcaaggcttctggatacaccttcaccgactactatatacactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcccggccagtagtaccccccaactactactacggtatggacgtctggggccaaggcaccctggtcaccgtctcctcactcgagggtggaggcggttcaggcggaggtggctctggcggtggcgctagcgaaacgacactcacgcagtctccagcattcatgtcagcgactccaggagacaaagtcaacatcccctgcaaagccagccaagacattgaggatgatatgaactggtaccaacagaaaccaggagaagctgctattttcattattcaagaagctactactctcgttcctggaatctcacctcgattcagtggcagcgggtatggaacagattttaccctcacaattaataacatagaatccgaggatgctgcatattacttctgtctacaacatgataatttcctccagggccaggggaccaagctggagatcaaacgt

SEQ ID NO: 52 - protein, 245 aa, Artificial Sequence
  2H6 Amino Acid Sequence
EVQLVQSGAEVKKPGASVKVSCKASGYTFTDYYIHWVRQAPGQGLEWMGIINPSGGSTSYAQKFQGRVTMTRDTSTSTVYMELSSLRSEDTAVYYCARPVVPPNYYYGMDVWGQGTLVTVSSLEGGGGSGGGGSGGGASETTLTQSPAFMSATPGDKVNIPCKASQDIEDDMNWYQQKPGEAAIFIIQEATTLVPGISPRFSGSGYGTDFTLTINNIESEDAAYYFCLQHDNFLQGQGTKLEIKR

SEQ ID NO: 53 - DNA, 738 bp, Artificial Sequence
  2H7 Nucleotide Sequence
gaagtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaattcccctcgcgtggtaggctatatgcttttgatatctggggccaagggacaatggtcaccgtctcttcactcgagggtggaggcggttcaggcggaggtggctctggcggtggcgctagcgacatccagatgacccagtccccctcttccgtgtctgcgtctgcaggagacagagtcaccatcacttgtcgggcgagtcaggatattagcagctggttagcctggtatcgacaggaactagggaaaccccctaaactcctgatctatgctgcatccagtttgcaaaggggagtcccatccaggttcagcggcagtggatctgggacagatttcactctcaccatcagcagcctgcagcctgaagattttggaatctactactgtcaacagtctaacagtttcccgtacaccttcggccaagggacacgactggagattaaacgt

SEQ ID NO: 54 - protein, 246 aa, Artificial Sequence
  2H7 Amino Acid Sequence
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKFPSRGRLYAFDIWGQGTMVTVSSLEGGGGSGGGGSGGGASDIQMTQSPSSVSASAGDRVTITCRASQDISSWLAWYRQELGKPPKLLIYAASSLQRGVPSRFSGSGSGTDFTLTISSLQPEDFGIYYCQQSNSFPYTFGQGTRLEIKR

SEQ ID NO: 55 - DNA, 747 bp, Artificial Sequence
  2H9 Nucleotide Sequence
caggtgcagctggtggagtctgggggaggcttggtacagcctggagggtccctgagactctcctgtgcagcctctggattcaccttcagtagttatgaaatgaactgggtccgccaggctccagggaaggggcttgagtgggtttcattcattactagtcgtgataatactatatactacgcagactctgtgaagggccgattcaccatctccagagacaattccaagaacacgctgtatctgcaaatggacagtctgagagccgaggacacggctgtgtattattgtacaagagtcttaaatggcctaagcggacactttgaccactggggccagggaaccctggtcaccgtctcctcactcgagggtggaggcggttcaggcggaggtggctctggcggtggcgctagcaattttatgctgactcagccccactctgtgtcgggttctccggggaaaacggtcaccatatcctgcaccggcagcagtggcagcattgccaggaactatgtgcagtggtaccagcagcgcccgggcagtgcccccaccactgtgatctatgaggataatcagagaccctctggggtccctgatcggttctctggctccatcgacagctcctccaactctgcctccctcaccatctctggactgaagactgaggacgaggctgactactactgtcagtcttatgataccagcattcattatgtcttcggaactgggaccaagctgaccgtcctaggt

SEQ ID NO: 56 - protein, 249 aa, Artificial Sequence
  2H9 Amino Acid Sequence
QVQLVESGGGLVQPGGSLRLSCAASGFTFSSYEMNWVRQAPGKGLEWVSFITSRDNTIYYADSVKGRFTISRDNSKNTLYLQMDSLRAEDTAVYYCTRVLNGLSGHFDHWGQGTLVTVSSLEGGGGSGGGGSGGGASNFMLTQPHSVSGSPGKTVTISCTGSSGSIARNYVQWYQQRPGSAPTTVIYEDNQRPSGVPDRFSGSIDSSSNSASLTISGLKTEDEADYYCQSYDTSIHYVFGTGTKLTVLG

SEQ ID NO: 57 - DNA, 744 bp, Artificial Sequence
  3A5 Nucleotide Sequence
gaagtgcagctggtggagtctgggggaggcgtggtccagcctgggaggtccctgaggctctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcccggatagcagtggctggtcgatcccaaaatgttgactactggggccagggaaccctggtcaccgtctcctcactcgagggtggaggcggttcaggcggaggtggctctggcggtggcgctagctcctatgagctgactcaggaccctgctgtgtctgtggccttgggacagacagtcaggatcacatgccaaggagacagcctcagaacctattatggaagttggtaccaacagaagccaggacaggcccctgcccttgtcatgtatggtagagacacccggccctcaggaatcccagaccgattctctgcctccagttcgagcaacacagcttccttgaccatcactggggctcaggcggaagatgaggctgactattggtgtagttcccgggacaccagtgttaaaaatggtgtggttttcggcggagggaccaaggtcaccgtcctaggt

SEQ ID NO: 58 - protein, 248 aa, Artificial Sequence
  3A5 Amino Acid Sequence
EVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARIAVAGRSQNVDYWGQGTLVTVSSLEGGGGSGGGGSGGGASSYELTQDPAVSVALGQTVRITCQGDSLRTYYGSWYQQKPGQAPALVMYGRDTRPSGIPDRFSASSSSNTASLTITGAQAEDEADYWCSSRDTSVKNGVVFGGGTKVTVLG

SEQ ID NO: 59 - DNA, 744 bp, Artificial Sequence
  3A12 Nucleotide Sequence
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactacgcagactccgtgaagggccgattcaccatctccagagacaacgccaagaacacgctgtatctgcaaatgaacagtctgagagccgaggacacggctgtatattactgtgcaagagtcgggggaagggattcttttgatatctggggccaaggaaccctggtcaccgtctcctcactcgagggtggaggcggttcaggcggaggtggctctggcggtggcgctagcgatattgtgatgactcagtctccactctccctgcccgtcacccttggacagccggcctccatctcctgcaggtctagtcagagcctcgtatacagtgatggaaacacctacttgaattggtttcagcagaggccaggccaatctccaaggcgcctaatttataaggtttctattcgggactctggggtcccagacagattcagcggcagtgggtcaggcactgatttcacactgaaaatcagcagggtggaggctgaggatgttggggtttattactgcatgcaatctacacactggcctccgacgttcggccaagggaccaaggtggaaatcaaacgt

SEQ ID NO: 60 - protein, 248 aa, Artificial Sequence
  3A12 Amino Acid Sequence
QVQLVESGGGVVQPGRSLRLSCAASGFTFSSYGMHWVRQAPGKGLEWVAVISYDGSNKYYADSVKGRFTISRDNAKNTLYLQMNSLRAEDTAVYYCARVGGRDSFDIWGQGTLVTVSSLEGGGGSGGGGSGGGASDIVMTQSPLSLPVTLGQPASISCRSSQSLVYSDGNTYLNWFQQRPGQSPRRLIYKVSIRDSGVPDRFSGSGSGTDFTLKISRVEAEDVGVYYCMQSTHWPPTFGQGTKVEIKR

SEQ ID NO: 61 - DNA, 732 bp, Artificial Sequence
  3B4 Nucleotide Sequence
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaaattagtgggtaactggaacttttacgactactggggccagggcaccctggtcaccgtctcctcactcgagggtggaggcggttcaggcggaggtggctctggcggtggcgctagcgatattgtgatgacccagtctccagccgccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttactactgtcagcagtataataactggcccaggacgttcggccaagggaccaaggtggaaatcaaacgt

SEQ ID NO: 62 - protein, 244 aa, Artificial Sequence
  3B4 Amino Acid Sequence
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCAKLVGNWNFYDYWGQGTLVTVSSLEGGGGSGGGGSGGGASDIVMTQSPAALSVSPGERATLSCRASQSVSSNLAWYQQKPGQAPRLLIYGASTRATGIPARFSGSGSGTEFTLTISSLQSEDFAVYYCQQYNNWPRTFGQGTKVEIKR

SEQ ID NO: 63 - DNA, 759 bp, Artificial Sequence
  3B11 Nucleotide Sequence
caggtgcagctggtggagtctgggggaggcgtggtccagcctgggaggtccctgagactctcctgtgcagcgtctggattcaccttcagtagctatggcatgcactgggtccgccaggctccaggcaaggggctggagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgagaatagcagtggctggctcatcacaagcgtcctactttgactactggggccagggaaccctggtcaccgtctcctcactcgagggtggaggcggttcaggcggaggtggctctggcggtggcgctagcgatattgtgatgacccagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcaccggcagtgggtcaggcactgatttcacactgaaaatcagcagggtggaggctgaagatgttggggtttattactgcatgcaaggtacacactggcccccaacgttcggccaagggaccaagctggagatcaaacgt

SEQ ID NO: 64 - protein, 253 aa, Artificial Sequence
  3B11 Amino Acid Sequence
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARIAVAGSSQASYFDYWGQGTLVTVSSLEGGGGSGGGGSGGGASDIVMTQSPLSLPVTLGQPASISCRSSQSLVYSDGNTYLNWFQQRPGQSPRRLIYKVSNRDSGVPDRFTGSGSGTDFTLKISRVEAEDVGVYYCMQGTHWPPTFGQGTKLEIKR

SEQ ID NO: 65 - DNA, 747 bp, Artificial Sequence
  3C4 Nucleotide Sequence
gaagtgcagctggtgcagtctggggctgaggtgaagaagcctggggcctcagtgaaggtttcctgcaaggcatctggatacaccttcaccagctactatatgcactgggtgcgacaggcccctggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgcgagaggcccccgaagtggccgatgggggtactggggccagggaaccctggtcaccgtctcctcactcgagggtggaggcggttcaggcggaggtggctctggcggtggcgctagcgacatccagatgacccagtctccagactccctggctgtgtctctgggcgagagggccaccatcaactgcaagtccagccagagtgttttatacacctccaacaataagaactacttagcttggtaccagcagaaaccaggacagcctcctaagctgctcatttactgggcatcgacccgggaatccggggtccctgaccgattcagtggcagcgggtctgggacagatttcactctcaccatcagcagcctgcaggctgaagatgtggcagtttatcactgtcagcaatattatagtcttcctatcaccttcggccaagggacacgactggagattaaacgt

SEQ ID NO: 66 - protein, 249 aa, Artificial Sequence
  3C4 Amino Acid Sequence
EVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGPRSGRWGYWGQGTLVTVSSLEGGGGSGGGGSGGGASDIQMTQSPDSLAVSLGERATINCKSSQSVLYTSNNKNYLAWYQQKPGQPPKLLIYWASTRESGVPDRFSGSGSGTDFTLTISSLQAEDVAVYHCQQYYSLPITFGQGTRLEIKR

SEQ ID NO: 67 - DNA, 756 bp, Artificial Sequence
  3C10 Nucleotide Sequence
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaataccttgcagactccgtgaagggccgattcaccatctccagagacaattccaagaacacgctgtatctgcaaatgaacagcctgagagctgaggacacggctgtgtattactgtgcgaagttggctagatattgtagtggtggtaggtgcccgtaccaccacggtatggacgtctggggccaaggcaccctggtcaccgtctcctcactcgagggtggaggcggttcaggcggaggtggctctggcggtggcgctagcgacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccactttgcaaagtggggtcccatcaaggttcagtggcagtggatctgggacagatttcactctcaccatcagcagtctgcaacctgaagattttgcaacttactactgtcaacagagttacagtaccccgatcaccttcggccaagggacacgactggagatcaaacgt

SEQ ID NO: 68 - protein, 252 aa, Artificial Sequence
  3C10 Amino Acid Sequence
QVQLVESGGGVVQPGRSLRLSCAASGFTFSSYGMHWVRQAPGKGLEWVAVISYDGSNKYLADSVKGRFTISRDNSKNTLYLQMNSLRAEDTAVYYCAKLARYCSGGRCPYHHGMDVWGQGTLVTVSSLEGGGGSGGGGSGGGASDIQMTQSPSSLSASVGDRVTITCRASQSISSYLNWYQQKPGKAPKLLIYAASTLQSGVPSRFSGSGSGTDFTLTISSLQPEDFATYYCQQSYSTPITFGQGTRLEIKR

SEQ ID NO: 69 - DNA, 741 bp, Artificial Sequence
  3D2 Nucleotide Sequence
gaagtgcagctggtggagtcagggggtggcgtggtccagcctgggaggtccctgagactctcctgtgcagcgtctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgagaaccgggcgatctgcttttgatatctggggccaagggacaatggtcaccgtctcttcactcgagggtggaggcggttcaggcggaggtggctctggcggtggcgctagcgatattgtgatgacccagtctccactctccctgcccgtcacccttggacagccggcctccatctcctgcaggtctagtcaaagcctcgtatacagtgatggaaacacctacttgaattggtttcagcagaggccaggccaatctccaaggcgcctaatttataaggtttctaaccgggactctggggtcccagacagattcagcggcagtgggtcaggcacttatttcacactggaaatcagcagggtggaggctgaggatgttggagtttattactgcatgcaaggtacacactggccgctcactttcggcggagggaccaaagtggatatcaaacgt

SEQ ID NO: 70 - protein, 247 aa, Artificial Sequence
  3D2 Amino Acid Sequence
EVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARTGRSAFDIWGQGTMVTVSSLEGGGGSGGGGSGGGASDIVMTQSPLSLPVTLGQPASISCRSSQSLVYSDGNTYLNWFQQRPGQSPRRLIYKVSNRDSGVPDRFSGSGSGTYFTLEISRVEAEDVGVYYCMQGTHWPLTFGGGTKVDIKR

SEQ ID NO: 71 - DNA, 744 bp, Artificial Sequence
  3D6 Nucleotide Sequence
caggtgcagctggtggagtctgggggaggcgtggtccagcctgggaggtccctgagactctcctgtgcagcgtctggattcagcttcagtaactatggcatgcactgggtccgccaggctccaggcaaggggctggagtgggtggcagttatatcatatgatggaagtaaggaatactatgtagactccgtgaagggccgattcaccatcttcagagacaattccaagaacaccctgtatctgcaaatgaacagcctgagagccgaggacacggctgtgtattactgtgcgaaactagcaattgggtcaccaggtgacgacgactactggggccagggaaccctggtcaccgtctcctcactcgagggtggaggcggttcaggcggaggtggctctggcggtggcgctagccagtctgagctgactcagcctccctccgcgtccgggtctcctggacagtcagtcaccatctcctgcactggaaccagcagtgacgttggtggttataactatgtctcctggtaccaacagcacccaggcaaagcccccaaactcatgatttatgaggtcagtaggcggccctcaggggtccctgatcgcttctctggctccaagtctggcaacacggcctccctgaccgtctctgggctccaggctgaggatgaggctgattattactgcagctcatatgcaggcagcaacaccgtggtattcggcggagggacccagctcaccgttttaggt

SEQ ID NO: 72 - protein, 248 aa, Artificial Sequence
  3D6 Amino Acid Sequence
QVQLVESGGGVVQPGRSLRLSCAASGFSFSNYGMHWVRQAPGKGLEWVAVISYDGSKEYYVDSVKGRFTIFRDNSKNTLYLQMNSLRAEDTAVYYCAKLAIGSPGDDDYWGQGTLVTVSSLEGGGGSGGGGSGGGASQSELTQPPSASGSPGQSVTISCTGTSSDVGGYNYVSWYQQHPGKAPKLMIYEVSRRPSGVPDRFSGSKSGNTASLTVSGLQAEDEADYYCSSYAGSNTVVFGGGTQLTVLG

SEQ ID NO: 73 - DNA, 753 bp, Artificial Sequence
  3H8 Nucleotide Sequence
caggtgcagctggtggagtctgggggaggcgaggtccagccagggaggtccctgagactctcctgtgcagcctctggattcaccttcagtagctatggcaggcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgagactttactatggttcgggggtgctggggaacggtatggacgtctggggccaaggcaccctggtcaccgtctcctcactcgagggtggaggcggttcaggcggaggtggctctggcggtggcgctagccagtctgtgctgactcagccaccctcagcgtctgggacccccgggcagagggtcaccatctcttgttctggaagcagctccgacatcggaagtaatactgtaaactggtaccagcaactcccaggaacggcccccaaactcctcatctacaataataatcagcggccctcaggggtccctgaccgattctctggctccaaagatgcttcggccaatgcagggattttactcatctctgggctccagtctgacgatgagggtgactattattgtatgatttggcacagcagcgcttgggtgttcggcggagggacccagctcaccgttttaggt

SEQ ID NO: 74 - protein, 251 aa, Artificial Sequence
  3H8 Amino Acid Sequence
QVQLVESGGGEVQPGRSLRLSCAASGFTFSSYGRHWVRQAPGKGLEWVAVISYDGSNKYYADSVKGRFTISRDNSKNTLYLQMNSLRAEDTAVYYCARLYYGSGVLGNGMDVWGQGTLVTVSSLEGGGGSGGGGSGGGASQSVLTQPPSASGTPGQRVTISCSGSSSDIGSNTVNWYQQLPGTAPKLLIYNNNQRPSGVPDRFSGSKDASANAGILLISGLQSDDEGDYYCMIWHSSAWVFGGGTQLTVLG

SEQ ID NO: 75 - DNA, 741 bp, Artificial Sequence
  3H11 Nucleotide Sequence
gaggtgcagctggtggagtctgggggaggcgtggtccagcctgggaggtccctgagactctcctgtgcagcctctggattcaccttcagtagctatggcatacactgggtccgccaggctccaggcaaggggctggagtgggtggcagttatatcatatgatggaagtaataaatactacgcagactccgtgaagggccgattcaccatctccagagacaattccaagaacacgctgtatctgcaaatgaacagcctgagagctgaggacacggctgtgtattactgtgcgagataccctaccagcggtagtagttattacgtgaatgactactggggccagggcaccctggtcaccgtctcctcactcgagggtggaggcggttcaggcggaggtggctctggcggtggcgctagcgacatccagatgacccagtctccatccttcctgtctgcatctgtaggagacagagtcaccatcacttgccgggcaagtcagagcattagcagctatttaaattggtatcagcagaaaccagggaaagcccctaagctcctgatctatgctgcatccaggttgcaaagtgaggtcccgtcaaggttcagtggcagtggatctgggacagatttcactctcaccatcagcagtctgcaacctgaagactttgcaacttactactgtcaacagagttacagaaccccgctcactttcggcggagggaccaaggtggagatcaaacgt

SEQ ID NO: 76 - protein, 247 aa, Artificial Sequence
  3H11 Amino Acid Sequence
EVQLVESGGGVVQPGRSLRLSCAASGFTFSSYGIHWVRQAPGKGLEWVAVISYDGSNKYYADSVKGRFTISRDNSKNTLYLQMNSLRAEDTAVYYCARYPTSGSSYYVNDYWGQGTLVTVSSLEGGGGSGGGGSGGGASDIQMTQSPSFLSASVGDRVTITCRASQSISSYLNWYQQKPGKAPKLLIYAASRLQSEVPSRFSGSGSGTDFTLTISSLQPEDFATYYCQQSYRTPLTFGGGTKVEIKR

SEQ ID NO: 77 - protein, 30 aa, Artificial Sequence
  Pga31 Peptide Sequence
QPLNVGNTVLQLGGSGDGTKVDIAEDGTLS

SEQ ID NO: 78 - protein, 28 aa, Artificial Sequence
  Utr2 Peptide Sequence
WPGGDSSNAKGTIEWAGGLINWDSEDIK

SEQ ID NO: 79 - protein, 32 aa, Artificial Sequence
  Phr2 Peptide Sequence
QDAGIYVIADLSQPDESINRDDPSWDLDLFER

SEQ ID NO: 80 - protein, 26 aa, Artificial Sequence
  Cht2 Peptide Sequence
LGKTVLLSLGGGVGDYGFSDVASATK

SEQ ID NO: 81 - protein, 352 aa, Artificial Sequence
  Pga31 Amino Acid Sequence
MKFHMRLQKKIFVLEYYIKPDISSFSGKYLFLLFFLFQSHINQLFDYIYFIQKYLICYIMKFLTAASLLTLSSSALAAIKDIQLYAQSSNNEVNDFGISSRHEGAALNYLFLAAPGVAENLKYDDETKTVYTELKAGSSTVRQPLNVGNTVLQLGGSGDGTKVDIAEDGTLSFDGSDSVGAAKNINDPYNYSKDSYAVVKGGDGAIPIKLVAKFTGDDKESASSSSSSAAPEPTASSSEAPKETPVYSNSTVTLYTTYCPLSTTITLTVCSDVCTPTVIETSGSVTVSSVQVPSKTASSEAAPPKTTVDSVSKPAPSGKKPTAAVTSFEGAANALTGGSVAIAVAAAIGLVF

SEQ ID NO: 82 - protein, 470 aa, Artificial Sequence
  Utr2 Amino Acid Sequence
MRFSTLHFAFLATLSSIFTVVAASDTTTCSSSKHCPEDKPCCSQFGICGTGAYCLGGCDIRYSYNLTACMPMPRMSTFQESFDSKDKVKEIELQSDYLGNSTEADWVYTGWVDYYDNSLLIQMPNHTTGTVVSSTKYLWYGKVGATLKTSHDGGVVTAFILFSDVQDEIDYEFVGYNLTNPQSNYYSQGILNYNNSRNSSVNNTFEYYHNYEMDWTEDKIEWYIDGEKVRTLNKNDTWNETSNRYDYPQTPSRIQFSLWPGGDSSNAKGTIEWAGGLINWDSEDIKKYGYYYAHIKEIYATAYDIPNDVKLDGNSTKESDYHAFLYNSTDGDASNIMLTTKKTWLGSDDATGFDPQNDDEDSSSNKAQETTITSVSGSSTITSVKTDSTKKTANVPAQNTAAAAQATAKSSTGTNTYDPSAGVGGFVQDSKSTDSGSSGSSSQGVANSLNESVISGIFASICLGILSFFM

SEQ ID NO: 83 - protein, 544 aa, Artificial Sequence
  Phr2 Amino Acid Sequence
MLLKSLFPSILAATSFVSSVAAEDLPAIEIVGNKFFYSNNGSQFYIKGIAYQQNNLDSNESFVDPLANPEHCKRDIPYLEAVDTNVIRVYALDTSQDHTECMQMLQDAGIYVIADLSQPDESINRDDPSWDLDLFERYTSVVDLFHNYTNILGFFAGNEVTNKKSNTDASAFVKAAIRDTKAYIKSKGYRSIPVGYSANDDSAIRVSLADYFACGDEDEAADFFGINMYEWCGDSSYKASGYESATNDYKNLGIPIFFSEYGCNEVRPRKFTEVATLFGDQMTPVWSGGIVYMYFEEENNYGLVSIKDNTVSTLKDYSYYSSEIKDIHPSSAKASAESASSISRTTCPTNTNNWEASTNLPPTPDKEVCECMSASLKCVVDDKVDSDDYSDLFSYICAKIDCDGINANGTTGEYGAYSPCHSKDKLSFVMNLYYEQNKESKSACDFGGSASLQSAKTASSCSAYLSSAGSSGLGTVSGTVRTDTSQSTSDSGSGSSSSSSSSSSSSSSGSSGSKSAASIVSVNLLTKIATIGISIVVGFGLITM

SEQ ID NO: 84 - protein, 584 aa, Artificial Sequence
  Cht2 Amino Acid Sequence
MLSFKSLLAAAVVASSALASASNQVALYWGQNGAGGQERLAQYCQETDVDIVLLSFLNLFPDPLNVNFANQCGNTFESGLLHCSQIGADIKTCQSLGKTVLLSLGGGVGDYGFSDVASATKFADTLWNKFGAGEDPERPFDDAVVDGFDFDIEHGGATGYPELATALRGKFAKDTSKNYFLSAAPQCPYPDASLGDLLSKVPLDFAFIQFYNNYCSINGQFNYDTWSKFADSAPNKNIKLFVGVPATSNIAGYVDTSKLSSAIEEIKCDSHFAGVSLWDASGAWLNTDEKGENFVVQVKNVLNQNACVAPSSSATTQSTTTTSSAVTQSTTTTSAAITQSATTTSAAVTTKSNQIVTSSSSSSSSIFYGNSTTESSTGIATGFTVLPTGSNENAATTGSGSNTKLAISTVTDVQKTVITITSCSEHKCVATPVTTGVVVVTDIDTVYTTYCPLTNSQVYVPVQTVVCTEETCVPSPTSTAQKPKASTTIKGVEKGQTTSYPVVGTTEGVKKIVTTSAQTVGSSTKYVTIELTSTITPVTYPTSVASNGTNTTVPVFTFEGGAAVANSLNSVWFPVPFLLAAFAF

SEQ ID NO: 85 - protein, 5 aa, Artificial Sequence
  VH - CDR1
SYGMH

SEQ ID NO: 86 - protein, 5 aa, Artificial Sequence
  VH - CDR1
SYAMS

SEQ ID NO: 87 - protein, 5 aa, Artificial Sequence
  VH - CDR1
SYAMH

SEQ ID NO: 88 - protein, 5 aa, Artificial Sequence
  VH - CDR1
SYAIS

SEQ ID NO: 89 - protein, 5 aa, Artificial Sequence
  VH - CDR1
SYGVH

SEQ ID NO: 90 - protein, 5 aa, Artificial Sequence
  VH - CDR1
NYDIY

SEQ ID NO: 91 - protein, 5 aa, Artificial Sequence
  VH - CDR1
SYWIG

SEQ ID NO: 92 - protein, 5 aa, Artificial Sequence
  VH - CDR1
NYGIN

SEQ ID NO: 93 - protein, 5 aa, Artificial Sequence
  VH - CDR1
GYYMH

SEQ ID NO: 94 - protein, 5 aa, Artificial Sequence
  VH - CDR1
SYGIS

SEQ ID NO: 95 - protein, 5 aa, Artificial Sequence
  VH - CDR1
GVGVG

SEQ ID NO: 96 - protein, 5 aa, Artificial Sequence
  VH - CDR1
DYYIH

SEQ ID NO: 97 - protein, 5 aa, Artificial Sequence
  VH - CDR1
SYEMN

SEQ ID NO: 98 - protein, 5 aa, Artificial Sequence
  VH - CDR1
SYYMH

SEQ ID NO: 99 - protein, 5 aa, Artificial Sequence
  NYGMH
NYGMH

SEQ ID NO: 100 - protein, 5 aa, Artificial Sequence
  VH - CDR1
SYGRH

SEQ ID NO: 101 - protein, 5 aa, Artificial Sequence
  VH - CDR1
SYGIH

SEQ ID NO: 102 - protein, 17 aa, Artificial Sequence
  VH - CDR2
VISYDGSNKYYADSVKG

SEQ ID NO: 103 - protein, 17 aa, Artificial Sequence
  VH - CDR2
TVSGSGGSTYYADSVKG

SEQ ID NO: 104 - protein, 17 aa, Artificial Sequence
  VH - CDR2
WINAGNGNTKYSQKFQG

SEQ ID NO: 105 - protein, 17 aa, Artificial Sequence
  VH - CDR2
GISWNSGSIGYADSVKG

SEQ ID NO: 106 - protein, 17 aa, Artificial Sequence
  VH - CDR2
IINPSGGSTSYAQKFQG

SEQ ID NO: 107 - protein, 17 aa, Artificial Sequence
  VH - CDR2
GISYDGNNKYYTDSVKG

SEQ ID NO: 108 - protein, 17 aa, Artificial Sequence
  VH - CDR2
WINAGNDNTKYSQKFQG

SEQ ID NO: 109 - protein, 17 aa, Artificial Sequence
  VH - CDR2
FINPKTGKTGYAQKFQG

SEQ ID NO: 110 - protein, 17 aa, Artificial Sequence
  VH - CDR2
IIYPGDSDTRYSPSFQG

SEQ ID NO: 111 - protein, 17 aa, Artificial Sequence
  VH - CDR2
AISGSGGSTYYADSVKG

SEQ ID NO: 112 - protein, 17 aa, Artificial Sequence
  VH - CDR2
VISYAGSNKYYADSVKG

SEQ ID NO: 113 - protein, 17 aa, Artificial Sequence
  VH - CDR2
RINPNSGDTNYAQKFQG

SEQ ID NO: 114 - protein, 17 aa, Artificial Sequence
  VH - CDR2
WISAYNGNTNYAQKLQG

SEQ ID NO: 115 - protein, 16 aa, Artificial Sequence
  VH - CDR2
LIYRNEDKRYSPSLER

SEQ ID NO: 116 - protein, 17 aa, Artificial Sequence
  VH - CDR2
RINPNSGGTNYAQKFQG

SEQ ID NO: 117 - protein, 17 aa, Artificial Sequence
  VH - CDR2
FITSRDNTIYYADSVKG

SEQ ID NO: 118 - protein, 17 aa, Artificial Sequence
  VH - CDR2
VISYDGSNKYLADSVKG

SEQ ID NO: 119 - protein, 17 aa, Artificial Sequence
  VH - CDR2
VISYDGSKEYYVDSVKG

SEQ ID NO: 120 - protein, 10 aa, Artificial Sequence
  VH - CDR3
DRSGWGSLDY

SEQ ID NO: 121 - protein, 5 aa, Artificial Sequence
  VH - CDR3
GYFDL

SEQ ID NO: 122 - protein, 11 aa, Artificial Sequence
  VH - CDR3
GGAAAGYYMDV

SEQ ID NO: 123 - protein, 6 aa, Artificial Sequence
  VH - CDR3
KYGMDV

SEQ ID NO: 124 - protein, 17 aa, Artificial Sequence
  VH - CDR3
IAHPRTRGGKEVEYFQH

SEQ ID NO: 125 - protein, 13 aa, Artificial Sequence
  VH - CDR3
VRRSGMARGLIDY

SEQ ID NO: 126 - protein, 12 aa, Artificial Sequence
  VH - CDR3
IAVAGRSQNVDY

SEQ ID NO: 127 - protein, 12 aa, Artificial Sequence
  VH - CDR3
GTKGKDYYYMDV

SEQ ID NO: 128 - protein, 7 aa, Artificial Sequence
  VH - CDR3
GRLWIDY

SEQ ID NO: 129 - protein, 7 aa, Artificial Sequence
  VH - CDR3
GTYYIDV

SEQ ID NO: 130 - protein, 4 aa, Artificial Sequence
  VH - CDR3
ISGY

SEQ ID NO: 131 - protein, 10 aa, Artificial Sequence
  VH - CDR3
LKTGDQLPDI

SEQ ID NO: 132 - protein, 8 aa, Artificial Sequence
  VH - CDR3
PIRGVKQH

SEQ ID NO: 133 - protein, 9 aa, Artificial Sequence
  VH - CDR3
LSRENAFDI

SEQ ID NO: 134 - protein, 6 aa, Artificial Sequence
  VH - CDR3
GQRMDV

SEQ ID NO: 135 - protein, 8 aa, Artificial Sequence
  VH - CDR3
DRDRGMDV

SEQ ID NO: 136 - protein, 18 aa, Artificial Sequence
  VH - CDR3
DSGYFDWFPYYYYYGMDV

SEQ ID NO: 137 - protein, 12 aa, Artificial Sequence
  VH - CDR3
AHRAATAAVLDY

SEQ ID NO: 138 - protein, 16 aa, Artificial Sequence
  VH - CDR3
LLGYSSGWYRPGAFDI

SEQ ID NO: 139 - protein, 17 aa, Artificial Sequence
  VH - CDR3
VDYDILTGYYPPQDMDV

SEQ ID NO: 140 - protein, 13 aa, Artificial Sequence
  VH - CDR3
LATTVTTLNAFDI

SEQ ID NO: 141 - protein, 17 aa, Artificial Sequence
  VH - CDR3
GRIAARRGDYYYYGMDV

SEQ ID NO: 142 - protein, 22 aa, Artificial Sequence
  VH - CDR3
EGRDIVVVVAATWDYYYYGMDV

SEQ ID NO: 143 - protein, 16 aa, Artificial Sequence
  VH - CDR3
MYNWNQRGGIHDAFDI

SEQ ID NO: 144 - protein, 13 aa, Artificial Sequence
  VH - CDR3
PVVPPNYYYGMDV

SEQ ID NO: 145 - protein, 12 aa, Artificial Sequence
  VH - CDR3
FPSRGRLYAFDI

SEQ ID NO: 146 - protein, 11 aa, Artificial Sequence
  VH - CDR3
VLNGLSGHFDH

SEQ ID NO: 147 - protein, 9 aa, Artificial Sequence
  VH - CDR3
VGGRDSFDI

SEQ ID NO: 148 - protein, 10 aa, Artificial Sequence
  VH - CDR3
LVGNWNFYDY

SEQ ID NO: 149 - protein, 14 aa, Artificial Sequence
  VH - CDR3
IAVAGSSQASYFDY

SEQ ID NO: 150 - protein, 9 aa, Artificial Sequence
  VH - CDR3
GPRSGRWGY

SEQ ID NO: 151 - protein, 18 aa, Artificial Sequence
  VH - CDR3
LARYCSGGRCPYHHGMDV

SEQ ID NO: 152 - protein, 8 aa, Artificial Sequence
  VH - CDR3
TGRSAFDI

SEQ ID NO: 153 - protein, 11 aa, Artificial Sequence
  VH - CDR3
LAIGSPGDDDY

SEQ ID NO: 154 - protein, 14 aa, Artificial Sequence
  VH - CDR3
LYYGSGVLGNGMDV

SEQ ID NO: 155 - protein, 13 aa, Artificial Sequence
  VH - CDR3
YPTSGSSYYVNDY

SEQ ID NO: 156 - protein, 17 aa, Artificial Sequence
  VL - CDR1
KSSQNVLYSSNNKNYLA

SEQ ID NO: 157 - protein, 11 aa, Artificial Sequence
  VL - CDR1
RASQSISSYLN

SEQ ID NO: 158 - protein, 13 aa, Artificial Sequence
  VL - CDR1
TRSSGSIASNYVQ

SEQ ID NO: 159 - protein, 13 aa, Artificial Sequence
  VL - CDR1
TGSSSNVANQGAT

SEQ ID NO: 160 - protein, 16 aa, Artificial Sequence
  VL - CDR1
RSSQSLVYSDGNTYLN

SEQ ID NO: 161 - protein, 11 aa, Artificial Sequence
  VL - CDR1
QGDILRSYYAS

SEQ ID NO: 162 - protein, 16 aa, Artificial Sequence
  VL - CDR1
RSSQSLLHSDGSTYLN

SEQ ID NO: 163 - protein, 11 aa, Artificial Sequence
  VL - CDR1
RASQSISTYLN

SEQ ID NO: 164 - protein, 11 aa, Artificial Sequence
  VL - CDR1
RASQSIARNLA

SEQ ID NO: 165 - protein, 16 aa, Artificial Sequence
  VL - CDR1
KSSQSLLHSDGKTYLY

SEQ ID NO: 166 - protein, 14 aa, Artificial Sequence
  VL - CDR1
TLRSGINVGTYRIY

SEQ ID NO: 167 - protein, 11 aa, Artificial Sequence
  VL - CDR1
QASQDIWKYVN

SEQ ID NO: 168 - protein, 17 aa, Artificial Sequence
  VL - CDR1
VSSQNLLFNSNKKNCLA

SEQ ID NO: 169 - protein, 17 aa, Artificial Sequence
  VL - CDR1
KSSHSVLYSSNNKNYLA

SEQ ID NO: 170 - protein, 14 aa, Artificial Sequence
  VL - CDR1
TGTSSDVGRYNYVS

SEQ ID NO: 171 - protein, 11 aa, Artificial Sequence
  VL - CDR1
KASQDIDDDMN

SEQ ID NO: 172 - protein, 13 aa, Artificial Sequence
  VL - CDR1
TRSSGNIASNYVQ

SEQ ID NO: 173 - protein, 11 aa, Artificial Sequence
  VL - CDR1
QGDSLRTYYAS

SEQ ID NO: 174 - protein, 11 aa, Artificial Sequence
  VL - CDR1
KASQDIEDDMN

SEQ ID NO: 175 - protein, 11 aa, Artificial Sequence
  VL - CDR1
RASQDISSWLA

SEQ ID NO: 176 - protein, 13 aa, Artificial Sequence
  VL - CDR1
TGSSGSIARNYVQ

SEQ ID NO: 177 - protein, 11 aa, Artificial Sequence
  VL - CDR1
QGDSLRTYYGS

SEQ ID NO: 178 - protein, 11 aa, Artificial Sequence
  VL - CDR1
RASQSVSSNLA

SEQ ID NO: 179 - protein, 17 aa, Artificial Sequence
  VL - CDR1
KSSQSVLYTSNNKNYLA

SEQ ID NO: 180 - protein, 14 aa, Artificial Sequence
  VL - CDR1
TGTSSDVGGYNYVS

SEQ ID NO: 181 - protein, 13 aa, Artificial Sequence
  VL - CDR1
SGSSSDIGSNTVN

SEQ ID NO: 182 - protein, 7 aa, Artificial Sequence
  VL - CDR2
WASTRES

SEQ ID NO: 183 - protein, 7 aa, Artificial Sequence
  VL - CDR2
AASSLQS

SEQ ID NO: 184 - protein, 7 aa, Artificial Sequence
  VL - CDR2
DDNQRPS

SEQ ID NO: 185 - protein, 7 aa, Artificial Sequence
  VL - CDR2
RNNNRPS

SEQ ID NO: 186 - protein, 7 aa, Artificial Sequence
  VL - CDR2
KVSNRDS

SEQ ID NO: 187 - protein, 7 aa, Artificial Sequence
  VL - CDR2
GKNNRPS

SEQ ID NO: 188 - protein, 7 aa, Artificial Sequence
  VL - CDR2
KVSRRDS

SEQ ID NO: 189 - protein, 7 aa, Artificial Sequence
  VL - CDR2
GASNLQS

SEQ ID NO: 190 - protein, 7 aa, Artificial Sequence
  VL - CDR2
GASTRAT

SEQ ID NO: 191 - protein, 7 aa, Artificial Sequence
  VL - CDR2
EDNQRPS

SEQ ID NO: 192 - protein, 7 aa, Artificial Sequence
  VL - CDR2
EVSNRFS

SEQ ID NO: 193 - protein, 11 aa, Artificial Sequence
  VL - CDR2
SISGSNYQQGS

SEQ ID NO: 194 - protein, 7 aa, Artificial Sequence
  VL - CDR2
WASTRKS

SEQ ID NO: 195 - protein, 7 aa, Artificial Sequence
  VL - CDR2
DVSNRPS

SEQ ID NO: 196 - protein, 7 aa, Artificial Sequence
  VL - CDR2
EATTLVP

SEQ ID NO: 197 - protein, 7 aa, Artificial Sequence
  VL - CDR2
DASNLDT

SEQ ID NO: 198 - protein, 7 aa, Artificial Sequence
  VL - CDR2
AASSLQR

SEQ ID NO: 199 - protein, 7 aa, Artificial Sequence
  VL - CDR2
GRDTRPS

SEQ ID NO: 200 - protein, 7 aa, Artificial Sequence
  VL - CDR2
KVSIRDS

SEQ ID NO: 201 - protein, 7 aa, Artificial Sequence
  VL - CDR2
AASTLQS

SEQ ID NO: 202 - protein, 7 aa, Artificial Sequence
  VL - CDR2
EVSRRPS

SEQ ID NO: 203 - protein, 7 aa, Artificial Sequence
  VL - CDR2
NNNQRPS

SEQ ID NO: 204 - protein, 7 aa, Artificial Sequence
  VL - CDR2
AASRLQS

SEQ ID NO: 205 - protein, 9 aa, Artificial Sequence
  VL - CDR3
QQYYSTPRT

SEQ ID NO: 206 - protein, 9 aa, Artificial Sequence
  VL - CDR3
QQTYTTPLT

SEQ ID NO: 207 - protein, 9 aa, Artificial Sequence
  VL - CDR3
QSYDSSIVV

SEQ ID NO: 208 - protein, 11 aa, Artificial Sequence
  VL - CDR3
SAWDSSLSAWV

SEQ ID NO: 209 - protein, 9 aa, Artificial Sequence
  VL - CDR3
MQGTHWPPS

SEQ ID NO: 210 - protein, 11 aa, Artificial Sequence
  VL - CDR3
NSRDSSGNHKV

SEQ ID NO: 211 - protein, 10 aa, Artificial Sequence
  VL - CDR3
MQGTRWPPIT

SEQ ID NO: 212 - protein, 9 aa, Artificial Sequence
  VL - CDR3
QQSYSTPLT

SEQ ID NO: 213 - protein, 9 aa, Artificial Sequence
  VL - CDR3
QQYETLPYT

SEQ ID NO: 214 - protein, 9 aa, Artificial Sequence
  VL - CDR3
QSYDSSNQV

SEQ ID NO: 215 - protein, 9 aa, Artificial Sequence
  VL - CDR3
MQTIQLPAT

SEQ ID NO: 216 - protein, 9 aa, Artificial Sequence
  VL - CDR3
MIWHSSAVV

SEQ ID NO: 217 - protein, 9 aa, Artificial Sequence
  VL - CDR3
QQSYSMPRT

SEQ ID NO: 218 - protein, 9 aa, Artificial Sequence
  VL - CDR3
QQSYSTPIT

SEQ ID NO: 219 - protein, 9 aa, Artificial Sequence
  VL - CDR3
QRSYSSPFT

SEQ ID NO: 220 - protein, 10 aa, Artificial Sequence
  VL - CDR3
QSYDSVNRGI

SEQ ID NO: 221 - protein, 9 aa, Artificial Sequence
  VL - CDR3
QQYYSIPFT

SEQ ID NO: 222 - protein, 10 aa, Artificial Sequence
  VL - CDR3
SSYTSSSTRV

SEQ ID NO: 223 - protein, 9 aa, Artificial Sequence
  VL - CDR3
LQHDNFPYT

SEQ ID NO: 224 - protein, 9 aa, Artificial Sequence
  VL - CDR3
QQFNSYPRT

SEQ ID NO: 225 - protein, 9 aa, Artificial Sequence
  VL - CDR3
QSYDSSTRV

SEQ ID NO: 226 - protein, 10 aa, Artificial Sequence
  VL - CDR3
QETHSVPPCN

SEQ ID NO: 227 - protein, 13 aa, Artificial Sequence
  VL - CDR3
NSRDSSVNRRDVV

SEQ ID NO: 228 - protein, 7 aa, Artificial Sequence
  VL - CDR3
LQHDNFL

SEQ ID NO: 229 - protein, 9 aa, Artificial Sequence
  VL - CDR3
QQSNSFPYT

SEQ ID NO: 230 - protein, 10 aa, Artificial Sequence
  VL - CDR3
QSYDTSIHYV

SEQ ID NO: 231 - protein, 12 aa, Artificial Sequence
  VL - CDR3
SSRDTSVKNGVV

SEQ ID NO: 232 - protein, 9 aa, Artificial Sequence
  VL - CDR3
MQSTHWPPT

SEQ ID NO: 233 - protein, 9 aa, Artificial Sequence
  VL - CDR3
QQYNNWPRT

SEQ ID NO: 234 - protein, 9 aa, Artificial Sequence
  VL - CDR3
MQGTHWPPT

SEQ ID NO: 235 - protein, 9 aa, Artificial Sequence
  VL - CDR3
QQYYSLPIT

SEQ ID NO: 236 - protein, 9 aa, Artificial Sequence
  VL - CDR3
MQGTHWPLT

SEQ ID NO: 237 - protein, 10 aa, Artificial Sequence
  VL - CDR3
SSYAGSNTVV

SEQ ID NO: 238 - protein, 9 aa, Artificial Sequence
  VL - CDR3
MIWHSSAWV

SEQ ID NO: 239 - protein, 9 aa, Artificial Sequence
  VL - CDR3
QQSYRTPLT

SEQ ID NO: 240 - protein, 17 aa, Artificial Sequence
  Linker
LEGGGGSGGGGSGGGAS

SEQ ID NO: 241 - protein, 21 aa, Artificial Sequence
  Pga31 (XP_717105.1)
HEGAALNYLFLAAPGVAENLK

SEQ ID NO: 242 - protein, 20 aa, Artificial Sequence
  Pga31 (XP_717105.1)
QPLNVGNTVLQLGGSGDGTK

SEQ ID NO: 243 - protein, 21 aa, Artificial Sequence
  Pga31 (XP_717105.1)
VDIAEDGTLSFDGSDSVGAAK

SEQ ID NO: 244 - protein, 10 aa, Artificial Sequence
  Pga31 (XP_717105.1)
NINDPYNYSK

SEQ ID NO: 245 - protein, 11 aa, Artificial Sequence
  Utr2 (XP_721748.1)
MSTFQESFDSK

SEQ ID NO: 246 - protein, 15 aa, Artificial Sequence
  Utr2 (XP_721748.1)
IQFSLWPGGDSSNAK

SEQ ID NO: 247 - protein, 9 aa, Artificial Sequence
  Utr2 (XP_721748.1)
YGYYYAHIK

SEQ ID NO: 248 - protein, 14 aa, Artificial Sequence
  Utr2 (XP_721748.1)
EIYATAYDIPNDVK

SEQ ID NO: 249 - protein, 19 aa, Artificial Sequence
  Utr2 (XP_721748.1)
GTIEWAGGLINWDSEDIKK

SEQ ID NO: 250 - protein, 14 aa, Artificial Sequence
  Phr2 (XP_719043.1)
DIPYLEAVDTNVIR

SEQ ID NO: 251 - protein, 12 aa, Artificial Sequence
  Phr2 (XP_719043.1)
DDPSWDLDLFER

SEQ ID NO: 252 - protein, 16 aa, Artificial Sequence
  Phr2 (XP_719043.1)
VDSDDYSDLFSYICAK

SEQ ID NO: 253 - protein, 23 aa, Artificial Sequence
  Cht2 (XP_721807)
TVLLSLGGGVGDYGFSDVASATK

SEQ ID NO: 254 - protein, 8 aa, Artificial Sequence
  Cht2 (XP_721807)
FADTLWNK

SEQ ID NO: 255 - protein, 19 aa, Artificial Sequence
  Cht2 (XP_721807)
LFVGVPATSNIAGYVDTSK

SEQ ID NO: 256 - protein, 9 aa, Artificial Sequence
  Cht2 (XP_721807)
LSSAIEEIK